(19) United States
(12) Patent Application Publication
(10) Pub. No.: US 2023/0235011 A1
(43) Pub. Date: Jul. 27, 2023

(54) FC-CD80 FUSION PROTEIN AND CONJUGATES THEREOF AND THEIR USES

(71) Applicant: BEIJING BEYOND BIOTECHNOLOGY CO., LTD, Beijing (CN)

(72) Inventors: Pinliang HU, Beijing (CN); Jing ZOU, Beijing (CN); Weidong HONG, Beijing (CN); Yun HE, Beijing (CN); Lingyun SONG, Beijing (CN); Wendi YANG, Beijing (CN)

(21) Appl. No.: 17/999,794

(22) PCT Filed: May 25, 2021
(86) PCT No.: PCT/CN2021/095750

§ 371 Date: Nov. 23, 2022

(30) Foreign Application Priority Data
May 25, 2020 (CN) 202010453172.X

(51) Int. Cl. C07K 14/705; A61P 35/00

(52) U.S. Cl. CPC C07K 14/70532; A61P 35/00; C07K 2319/30

(57) ABSTRACT

A Fc-CD80 fusion protein has a CD80 extracellular domain (ECD) linked to a C-terminal of an immunoglobulin Fc domain. The Fc-CD80 fusion protein can be used for the preparation of a conjugate, the conjugate including the Fc-CD80 fusion protein as a first component, and a second component containing a second effector molecule, the second component being located at an N-terminal of the first component. In addition, a conjugate including the Fc-CD80 fusion protein, a pharmaceutical composition including the Fc-CD80 fusion protein and/or the conjugate are effective for the treatment or prevention of cancerous diseases in individuals.

### TECHNICAL FIELD

The present invention generally relates to the field of pharmaceutical biotechnology. Specifically, the present invention relates to a fusion protein in which a CD80 extracellular domain (ECD) is linked to a C-terminal of an immunoglobulin Fc domain, and use of the fusion protein for the treatment or prevention of cancerous diseases in an individual. The present invention further relates to a conjugate of the Fc-CD80 fusion protein, and use of the conjugate for the treatment or prevention of cancerous diseases in an individual, the conjugate including the Fc-CD80 fusion protein as a first component, and a second component containing a second effector molecule, the second component being located at an N-terminal of the first component.

### BACKGROUND ART

Recent studies have found that PD-1 inhibits T cell functions mainly by inactivating a CD80-CD28 signal transduction pathway after binding to PD-L1. A PD-L1 antibody drug or PD-1 antibody drug functions to remove this inhibition, but a PD-L1 antibody or PD-1 antibody has only an indirect activation effect on a B7-CD28 signal transduction pathway. Specifically, PD-1 rapidly recruits Shp2 phospholipase after binding to PD-L1, and the Shp2 phospholipase preferentially dephosphorylates CD28 and inhibits lymphocyte activation. This inhibition effect is stronger than an inhibition effect on a T cell receptor (TCR) (Hui E. et al., Science, 2017, 355(6332): 1428-1433). Kamphorst A O et al. also confirmed that the activation of CD28 co-stimulatory signals was one of the important conditions for “reactivation” of T cells (Kamphorst A O et al., Science, 2017, 355(6332): 1423-1427), and an inhibitory effect of the PD-1 antibody on tumors was significantly reduced if the binding of B7.1 (CD80) molecules to CD28 was blocked with an anti-B7.1 (CD80) antibody.

Unlike the PD1 antibody or PD-L1 antibody that acts indirectly on CD28, CD80 or CD86 can bind to CD28 directly, thereby activating CD28. This binding force is relatively low, and K_{D} is only 4 μM. In addition, CD80 can also bind to PD-L1 and CTLA-4 whose K_{D} is 1.7 μM and 0.2 μM, respectively (Butte M J., Immunity, 2007, 27(1): 111-122). The binding of CD80 to CD28 is regarded as a direct immune activation effect. The binding of CD80 to PD-L1 can exert the same effect as the PD-L1 antibody, preventing the interaction between PD-L1 and PD1. Although the binding of CD80 to CTLA-4 is an immunosuppressive effect that inhibits the activation of CD28, the CD80 fusion protein acts as a CTLA-4-trap (Horn L A, et al., Cancer Immunol Res. 2018 January; 6(1): 59-68) that inhibits an immunosuppressive function of CTLA-4. In summary, the CD80 immune fusion protein activates an immune system by directly binding CD28, PD-L1, and CTLA-4. More and more studies have shown that CD80 or CD86 fusion protein has a significant tumor growth inhibition effect (Horn L A, et al., Cancer Immunol Res. 2018; 6(1): 59-68; U.S. Pat. No. 8,956,619, U.S. patent Ser. No. 10/377,810, U.S. Pat. No. 9,650,429). In the studies of Liu A et al., soluble B7.1-Fc showed a co-stimulating activity of T cell proliferation in in vitro experiments, and CT26 tumors could be completely regressed after 5 days of treatment when 5 μg of B7.1-Fc was administered daily to each mouse in vivo experiments (Liu A, et al., Clin Cancer Res. 2005 Dec. 1; 11(23): 8492-502).

Several studies on CD80 (also known as B7.1) or CD86 (also known as B7.2) fusion proteins have been reported.

A CD80 immune fusion protein (also known as FTP155) developed by Five Prime Therapeutics, Inc. is formed by fusing CD80 extracellular regions (IgV and IgC functional regions) with Fc regions of IgG1, and in vivo research results have proved that mCD80-Fc has a better tumor inhibition effect than mPD-1 when used alone; and in addition, mCD80-Fc has a better synergistic effect with mPD-1 (WO/2017/079117A1; WO/2018/201014A1; https://www.fiveprime.com/programs/FPT155/). Five Prime Therapeutics, Inc. believes that the CD80-Fc fusion protein inhibits tumor growth through activation of an immune system as equally to, even better than GITRL, OX40L and 4-1BBL.

ALPN-202 is an immune fusion protein formed by a mutant of IgV in a CD80 extracellular region developed by Alpine Immune Sciences and IgG1 Fc, and the immune fusion protein retains and improves the binding ability of CD80 to CTLA-4, PD-L1 and CD28 (WO2017/181152). Like FTP155, ALPN-202 hinders the binding of PD-L1 to PD-1 after binding to PD-L1, thereby reducing the ability of PD-1 to inhibit the immune response and block the immune system; activates CD28 by “stepping on the accelerator” to enhance the immune response; and reduces, by binding to CTLA-4, the immunosuppressive capacity caused by the binding of CTLA-4 to CD28. ALPN-202 is expected to enter the Phase I trial in cancer patients in 2020 (WO/2018/170026A2, WO/2017/181152A2, WO/2018/170026A3, https://www.alpineimmunesciences.com/pipeline/oncology/).

It has been reported that B7-1 and B7-2 molecules are mainly involved in the interaction with their IgV domains at N-terminals when binding to CD28, CTAL-4 and PDL-1, and IgC mainly maintains the stability of B7-1 and B7-2 (Truneh Al, et al., Mol Immunol, 1996, 33:321-334; Kariv I, et al., J Immunol., 1996, 157: 29-38; Morton P A, et al., J Immunol., 1996 156: 1047-1054; Peach R J, et al., J Biol Chem., Sep. 8, 1995; 270(36):21181-7). Based on this cognition, many studies in the past have placed CD80 (B7.1) or CD86 (B7.2) at the N-terminal of a bifunctional fusion protein when constructing the bifunctional fusion protein of CD80 (B7.1) or CD86 (B7.2) to avoid interfering with the binding ability of IgV. However, such a structural design will interfere with the binding ability of a second functional molecule, which will inevitably affect the function of the bifunctional fusion protein. Challita-Eid P M, et al. believed that a bidirectional antibody constructed by fusing CD80 with a heavy chain N-terminal of a HER2 antibody could bind to CD28 and CTLA-4, and observed that the bidirectional antibody had an activation effect on T lymphocytes, but the affinity of the bidirectional antibody was decreased by 2.5 times compared to that of the HER2 antibody alone (Challita-Eid P M, et al., J. Immunol., 160(7): 3419-26 (1998). Liu A et al. constructed CD80 to a heavy chain N-terminal of a tumor necrosis therapy (TNT) NHS76 antibody to obtain a bifunctional antibody B7.1/NHS76 fusion protein, which had 35-55% inhibitory effects on tumors caused by Colon 26, RENCA and MAD109 cell lines implanted in mice, all of which were significantly stronger than that of the original NHS76 antibody. However, compared with the original NHS76 antibody, the binding ability of the bifunctional antibody B7.1/NHS76 to TNT was decreased by 13 times (Liu A, et al., J Immunother. 2006 July-August; 29(4):425-35).

Although the prior art has taught the CD80-Fc fusion protein, there is still a need for alternative fusion protein structure patterns with improved performances to meet the demands for newer and more effective treatments for cancers.

### SUMMARY OF THE INVENTION

The present invention discloses a CD80 fusion protein that differs from the prior art.

In a first aspect, the present invention provides an Fc-CD80 fusion protein. The Fc-CD80 fusion protein includes an immunoglobulin Fc domain and a CD80 extracellular domain, wherein the CD80 extracellular domain is optionally linked to the immunoglobulin Fc domain by a linking peptide, and is located at a C-terminal of the Fc domain. The inventor has unexpectedly found that the binding ability of the Fc-CD80 fusion protein to CD28, CTLA-4 and PD-L1 is higher than that of a CD80-Fc fusion protein to CD28, CTLA-4 and PD-L1.

In some embodiments, the immunoglobulin Fc domain in the Fc-CD80 fusion protein of the present invention is a human or mouse Fc domain, preferably, a Fc domain of human IgG1, IgG2, IgG3 or IgG4; and more preferably, the immunoglobulin Fc domain is a Fc domain of an amino acid sequence shown in SEQ ID NO: 8, 9 or 10, or a Fc domain having at least 90%, 91%, 92%, 93%, 94%, 95%, 96%, 97%, 98%, 99% or more sequence identity with the amino acid sequence shown in SEQ ID NO: 8, 9 or 10.

In some embodiments, a CD80 extracellular domain in the Fc-CD80 fusion protein of the present invention is human CD80 ECD; preferably, the human CD80 ECD includes human CD80 IgV or human CD80 IgVIgC; and more preferably, the human CD80 ECD has an amino acid sequence shown in SEQ ID NO: 1 or 2 or has at least 90%, 91%, 92%, 93%, 94%, 95%, 96%, 97%, 98%, 99% or more with the amino acid sequence shown in SEQ ID NO: 1 or 2.

In addition, with the studies on tumors, it has been recognized that a main mechanism of tumor escape is also related to a tumor microenvironment (TME), which consists of immunosuppressive cells (e.g., regulatory T cells (Tregs), tumor-associated macrophages (TAMs) and myeloid-derived suppressor cells (MDSCs), soluble factors, inhibitory molecules expressed on tumor cells or antigen-presenting cells, and an extracellular matrix.

This immunosuppressive tumor microenvironment not only promotes the tumor growth and migration, but also helps tumor cells evade the surveillance from host immunity and resist the immunotherapy. Considering that there are multiple signal transduction pathways involved in tumor initiation and progression, it is difficult for a single-target immunotherapy (e.g., treatment for PD1 or PD-L1) to achieve satisfactory therapeutic results. The present inventor further conjugates a second effector molecule (e.g., an antibody fragment, a receptor extracellular functional domain or cytokine) to the N-terminal of the Fc-CD80 fusion protein of the present invention, and the resulting conjugate retains the biological activity of CD80 and the secondary effector molecule, but also has the advantages of long biological half-life and easy purification.

Therefore, in a second aspect, the present invention provides use of the Fc-CD80 fusion protein of the present invention for the preparation of a conjugate, the conjugate including the Fc-CD80 fusion protein as a first component, and a second component containing a second effector molecule; the second effector molecule includes, but is not limited to, an antibody fragment (e.g., the antibody fragment is Fab, Fab′, F(ab′)₂, Fv, or single-chain Fv), a receptor extracellular functional domain (e.g., an extracellular functional domain of the following receptors: a vascular endothelial growth factor receptor (VEGFR), a transforming growth factor βII receptor, CD95, a lymphotoxin beta receptor, an interleukin-1 receptor accessory protein, 4-1BBL, Lag-3, activin A receptor type II-like 1 (ALK1), AITRL, an IL-15 receptor a (IL15RA), a frizzled class receptor 8 (FZD8), an activin receptor type IIB, an activin A receptor type IIA, GITR, OX40, CD24 or CD40), or other proteins (e.g., cytokines), and the second component is located at the N-terminal of the Fc-CD80 fusion protein.

In a third aspect, the present invention provides a conjugate, the conjugate including the Fc-CD80 fusion protein as a first component, and a second component containing a second effector molecule; the second effector molecule includes, but is not limited to, an antibody fragment (e.g., the antibody fragment is Fab, Fab′, F(ab′)₂, Fv, or single-chain Fv), a receptor extracellular functional domain (e.g., an extracellular functional domain of the following receptors: a vascular endothelial growth factor receptor (VEGFR), a transforming growth factor βII receptor, CD95, a lymphotoxin beta receptor, an interleukin-1 receptor accessory protein, 4-1BBL, Lag-3, activin A receptor type II-like 1 (ALK1), AITRL, an IL-15 receptor a (IL15RA), a frizzled class receptor 8 (FZD8), an activin receptor type IIB, an activin A receptor type IIA, GITR, OX40, CD24 or CD40), or other proteins (e.g., cytokines), and the second component is located at the N-terminal of the Fc-CD80 fusion protein.

In one embodiment, the conjugate of the present invention includes the Fc-CD80 fusion protein of the present 20 invention as a first component, and an anti-VEGF antibody fragment (e.g., bevacizumab antibody fragment) as a second component, wherein the second component is located at the N terminal of the Fc-CD80 fusion protein.

In one embodiment, the conjugate of the present invention includes the Fc-CD80 fusion protein of the present invention as a first component, and an anti-HER2 antibody fragment (e.g., trastuzumab antibody fragment), an anti-GPC-3 antibody fragment (e.g., codrituzumab antibody fragment), or an anti-trop-2 antibody fragment (e.g., sacituzumab antibody fragment) as a second component, wherein the second component is located at the N-terminal of the Fc-CD80 fusion protein.

In one embodiment, the conjugate of the present invention includes the Fc-CD80 fusion protein of the present invention as a first component, and a polypeptide containing an extracellular functional domain of a receptor (e.g., VEGFR, TGFβ II receptor) as a second component, wherein the second component is located at the N terminal of the Fc-CD80 fusion protein. In one embodiment, the second component in the conjugate of the present invention is an extracellular functional domain of the receptor (e.g., VEGFR, TGFβ II receptor). In yet one embodiment, the second component in the conjugate of the present invention is a Fab-like fragment formed by respectively connecting CH1 of an immunoglobulin heavy chain and CL of an immunoglobulin light chain to a C-terminal of the extracellular functional domain of the receptor (e.g., VEGFR, TGFβ II receptor).

In a fourth aspect, the present invention provides a pharmaceutical composition, the pharmaceutical composition including the Fc-CD80 fusion protein of the present invention and/or the conjugate of the present invention, preferably, the pharmaceutical composition further including a second therapeutic agent. The second therapeutic agent is any therapeutic agent that is favorably combined with the Fc-CD80 fusion protein of the present invention and/or the conjugate of the present invention.

In a fifth aspect, the present invention provides uses of the Fc-CD80 fusion protein of the present invention, the conjugate of the present invention or the pharmaceutical composition of the present invention for the preparation of drugs for the treatment or prevention of cancerous diseases (e.g., solid tumors and soft tissue tumors) in individuals. Preferably, the cancerous diseases are melanoma, breast cancer, colon cancer, esophageal cancer, gastrointestinal stromal tumor (GIST), kidney cancer (e.g., renal cell carcinoma), liver cancer, non-small cell lung cancer (NSCLC), ovarian cancer, pancreatic cancer, prostate cancer, head and neck tumors, gastric cancer, hematologic malignancies (e.g., lymphoma); in particular, the disease is liver cancer; and preferably, the individual is a mammal, more preferably a human.

### BRIEF DESCRIPTION OF THE DRAWINGS

The preferred embodiments of the present invention described in detail below will be better understood while reading together with the following accompanying drawings. For the purpose of illustrating the present invention, the present preferred embodiments are shown in the drawings. However, it should be understood that the present invention is not limited to the precise arrangement and means of embodiments shown in the drawings.

FIG. 1 illustrates a structure of an Fc-CD80 fusion protein of the present invention.

FIG. 2A illustrates a structure of a conjugate of the present invention.

FIG. 2B illustrates a structure of another conjugate of the present invention.

FIG. 3A shows the results of a fusion protein or conjugate prepared and purified in an example being subjected to SDS-PAGE electrophoresis in the presence of a reducing agent (5 mM 1,4-dithiothreitol) and stained with Coomassie blue. Lane 1: a protein molecular weight standard marker; Lane 2: a fusion protein BY24.23 (CD80-Fc); Lane 3: a fusion protein BY24.30 (Fc-CD80); Lane 4: a fusion protein BY24.24 (Fc-mCD80); Lane 5: a conjugate BY24.22 (VEGFR-Fc-CD80); Lane 6: a fusion protein 301-8 (VEGFR-Fc, aflibercept); Lane 7: a conjugate BY41.6.

FIG. 3B shows the results of a fusion protein or conjugate prepared and purified in an example being subjected to SDS-PAGE electrophoresis in the presence of a reducing agent (5 mM 1,4-dithiothreitol) and stained with Coomassie blue. Lane 1: a protein molecular weight standard marker; Lane 2: trastuzumab; Lane 3: a conjugate BY12.7 (trastuzumab-CD80); Lane 4: Bevacizumab; Lane 5: a conjugate BY24.26 (bevacizumab-CD80); Lane 6: an antibody BY20.2 (i.e., codrituzumab); Lane 7: a conjugate BY20.3 (codrituzumab-CD80); Lane 8: an antibody BY43 (i.e., sacituzumab); Lane 7: a conjugate BY43.2 (sacituzumab-CD80).

FIG. 3C shows the results of a fusion protein prepared and purified in an example being subjected to SDS-PAGE electrophoresis in the presence of and in the absence of a reducing agent (5 mM 1,4-dithiothreitol) and stained with Coomassie blue. Lane 1: a protein molecular weight standard marker; Lane 2: a fusion protein BY24.30 (Fc-CD80) under a reducing condition; Lane 3: a fusion protein BY24.23 (CD80-Fc) under a reducing condition; Lane 4: a fusion protein BY24.30 (Fc-CD80) under a non-reducing condition; Lane 5: a fusion protein BY24.23 (CD80-Fc) under a non-reducing condition.

FIG. 4A shows binding curves of a fusion protein BY24.30 (i.e., a Fc-hCD80 fusion protein) and a fusion protein BY24.23 (i.e., a hCD80-Fc fusion protein), which specifically bind to recombinant human PD-L1, as determined by ELISA. The abscissa represents a fusion protein concentration expressed in a logarithmic term; and the ordinate represents the absorbance at 450 nm.

FIG. 4B shows binding curves of a fusion protein BY24.30 (i.e., a Fc-hCD80 fusion protein) and a fusion protein BY24.23 (i.e., a hCD80-Fc fusion protein), which specifically bind to recombinant human CD28, as determined by ELISA. The abscissa represents a fusion protein concentration expressed in a logarithmic term; and the ordinate represents the absorbance at 450 nm.

FIG. 4C shows binding curves of a fusion protein BY24.30 (i.e., a Fc-hCD80 fusion protein) and a fusion protein BY24.23 (i.e., a hCD80-Fc fusion protein), which specifically bind to recombinant human CTLA-4, as determined by ELISA. The abscissa represents a fusion protein concentration expressed in a logarithmic term; and the ordinate represents the absorbance at 450 nm.

FIG. 5 shows growth inhibition effects of a fusion protein BY24.24 (Fc-mCD80) and an antibody mPD1 on mouse colon cancer CT26, respectively.

FIG. 6 shows tumor volumes (Tumor Volume (TV)) in a solvent group, an Opdivo group, a conjugate BY24.26 group and an Opdivo+BY24.26 group in a mouse model of human hepatoma cells HUH7. The abscissa represents the number of days after mice are inoculated with human hepatoma cells HUH7; and the ordinate represents the tumor volume.

FIG. 7A shows expression photos (magnification: 100×) of CD8⁺, CD4⁺, and CD31⁺ in a solvent group taken in a mouse model of human hepatoma cells HUH7.

FIG. 7B shows expression photos (magnification: 100×) of CD8⁺, CD4⁺, and CD31⁺ in an Opdivo group taken in a mouse model of human hepatoma cells HUH7.

FIG. 7C shows expression photos (magnification: 100×) of CD8⁺, CD4⁺, and CD31⁺ in a conjugate BY24.26 group taken in a mouse model of human hepatoma cells HUH7.

FIG. 7D shows expression photos (magnification: 100×) of CD8⁺, CD4⁺, and CD31⁺ in an Opdivo+BY24.26 group taken in a mouse model of human hepatoma cells HUH7.

FIG. 8 shows tumor volumes in a solvent group, an antibody BY20.2 group and a conjugate BY20.3 group in a mouse model of human hepatoma cells HUH7. The abscissa represents the number of days after mice are inoculated with human hepatoma cells HUH7; and the ordinate represents the tumor volume.

FIG. 9 shows tumor volumes in a solvent group, a conjugate BY24.22 (VEGFR-Fc-CD80) group and an conjugate BY41.6 group in a mouse model of human hepatoma cells HUH7. The abscissa represents the number of days after mice are inoculated with human hepatoma cells HUH7; and the ordinate represents the tumor volume.

FIG. 10A shows the crystal structure analysis (PDB ID: 118L) of CTLA-4 and CD80.

FIG. 10B shows a crystal structure of Fc (PDB ID: 3KYM).

FIG. 11 shows amino acid sequences of human CD80, human CD86, and human ICOSL.

FIG. 12 shows an amino acid sequence of mouse CD80.

FIG. 13 shows an amino acid sequence of a human CD80 extracellular domain IgV and an amino acid sequence of a human CD80 extracellular domain IgC.

FIG. 14 illustrates amino acid sequences of fusion proteins in a Fc region and a CD80 extracellular region.

FIG. 15 shows an amino acid sequence of an anti-HER2/neu antibody and CD80 bifunctional conjugate.

FIG. 16 shows an amino acid sequence of an anti-VEGF antibody and CD80 bifunctional conjugate.

FIG. 17 shows an amino acid sequence of an anti-CD20 antibody and CD80 bifunctional conjugate.

FIG. 18 shows an amino acid sequence of an anti-Trop-2 antibody and CD80 bifunctional conjugate.

FIG. 19 shows an amino acid sequence of an anti-PD1 antibody and CD80 bifunctional conjugate.

FIG. 20 shows an amino acid sequence of an anti-PD-L1 antibody and CD80 bifunctional conjugate.

FIG. 21 shows an amino acid sequence of an anti-glypican-3 antibody and CD80 bifunctional conjugate.

FIG. 22 shows an amino acid sequence of an anti-CTLA-4 antibody and CD80 bifunctional conjugate.

FIG. 23 shows an amino acid sequence of an anti-EGFR antibody and CD80 bifunctional conjugate.

FIG. 24 shows an amino acid sequence of an anti-ALK-1 antibody and CD80 bifunctional conjugate.

FIG. 25 shows an amino acid sequence of an anti-CD30 antibody and CD80 bifunctional conjugate.

FIG. 26 shows an amino acid sequence of an anti-CD33 antibody and CD80 bifunctional conjugate.

FIG. 27 shows an amino acid sequence of an anti-CEA antibody and CD80 bifunctional conjugate.

FIG. 28 shows an amino acid sequence of an anti-IGF1R antibody and CD80 bifunctional conjugate.

FIG. 29 shows an amino acid sequence of an anti-CD47 antibody and CD80 bifunctional conjugate.

FIG. 30 shows an amino acid sequence of an anti-TIM-3 antibody and CD80 bifunctional conjugate.

FIG. 31 shows an amino acid sequence of an anti-LAG-3 antibody and CD80 bifunctional conjugate.

FIG. 32 shows an amino acid sequence of an anti-TIGIT antibody and CD80 bifunctional conjugate.

FIG. 33 shows an amino acid sequence of an anti-4-1BB antibody and CD80 bifunctional conjugate.

FIG. 34 shows an amino acid sequence of an anti-OX40 antibody and CD80 bifunctional conjugate.

FIG. 35 shows an amino acid sequence of an anti-ICOS antibody and CD80 bifunctional conjugate.

FIG. 36 shows an amino acid sequence of an anti-CD27 antibody and CD80 bifunctional conjugate.

FIG. 37 shows an amino acid sequence of a transforming growth factor β II receptor extracellular region and CD80 extracellular region IgG-like bifunctional conjugate.

FIG. 38 shows an amino acid sequence of a CD24 extracellular region and CD80 extracellular region IgG-like bifunctional conjugate.

FIG. 39 shows an amino acid sequence of an ALK1 extracellular region and CD80 extracellular region IgG-like bifunctional conjugate.

FIG. 40 shows an amino acid sequence of an FZD8 extracellular region and CD80 extracellular region IgG-like bifunctional conjugate.

FIG. 41 shows an amino acid sequence of a CD47 extracellular region and CD80 extracellular region IgG-like bifunctional conjugate.

FIG. 42 shows an amino acid sequence of a VEGFR extracellular region and CD80 extracellular region bifunctional conjugate.

FIG. 43 shows an amino acid sequence of an interleukin 1 receptor accessory protein extracellular region and CD80 extracellular region bifunctional conjugate.

FIG. 44 shows an amino acid sequence of a CD95 protein extracellular region and CD80 extracellular region bifunctional conjugate.

FIG. 45 shows an amino acid sequence of an IL-2 and CD80 extracellular region bifunctional conjugate.

FIG. 46 shows an amino acid sequence of an IL-7 and CD80 extracellular region bifunctional conjugate.

FIG. 47 shows an amino acid sequence of an IL-33 and CD80 extracellular region bifunctional conjugate.

FIG. 48 shows an amino acid sequence of an IL-13 and CD80 extracellular region bifunctional conjugate.

FIG. 49 shows an amino acid sequence of an anti-CLDN18.2 antibody and CD80 bifunctional conjugate.

FIG. 50 shows an amino acid sequence of an anti-IL-17 antibody and aCD80 bifunctional conjugate.

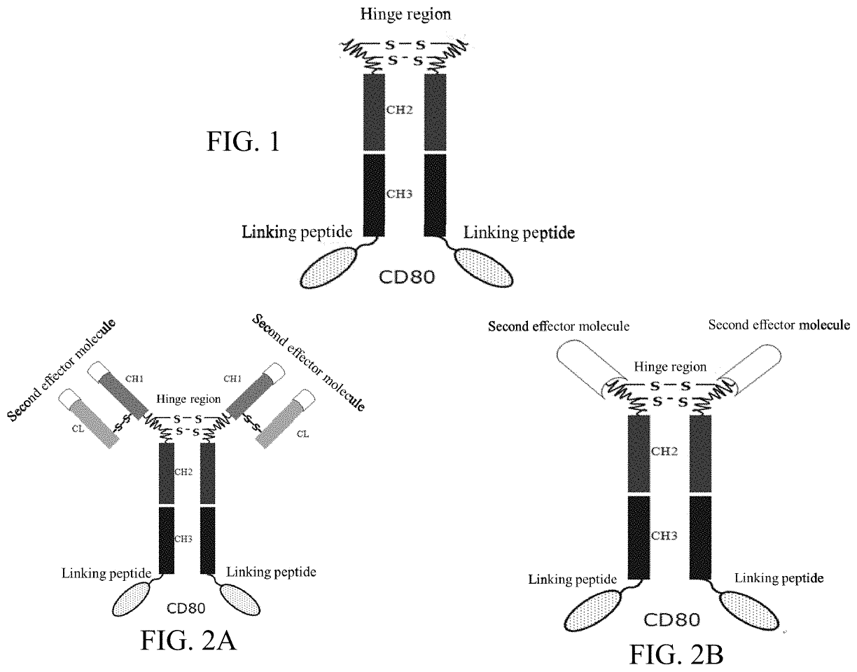

### DETAILED DESCRIPTION OF THE INVENTION

The present invention provides an Fc-CD80 fusion protein and a conjugate containing the Fc-CD80 fusion protein, a pharmaceutical composition including the Fc-CD80 fusion protein, and a pharmaceutical composition including the conjugate.

The present invention further provides a method for producing an Fc-CD80 fusion protein and a conjugate containing the Fc-CD80 fusion protein, and uses of the Fc-CD80 fusion protein and the conjugate containing the Fc-CD80 fusion protein for the treatment or prevention of cancerous diseases in individuals.

Unless otherwise defined below, the terms used in this specification are as commonly used in the art.

### I. Definition

The term “approximate” when used in conjunction with numeric values means to cover numeric values in a range that has a lower limit of 5% less than a specified numeric value and an upper limit of 5% greater than the specified numeric value.

As used herein, the term “comprising” or “including” means including the described elements, integers, or steps, but does not exclude any other features, integers, or steps.

The terms “PD-1/PD-L1 inhibitory signal transduction pathway”, “PD-1/PD-L1 signal transduction pathway”, “PD-1/PD-L1 signal transduction pathway”, and “PD-1/PD-L1 pathway” are used interchangeably herein and refer to any intracellular signal transduction pathway initiated by the binding of PD-1 to PD-L1.

The terms “mitigate”, “interfere”, “inhibit” or “block” PD-1/PD-L1 inhibitory signal transduction pathways, as used herein, are used interchangeably and refer to (i) interfering with the interaction between PD-1 and PD-L1; and/or (ii) inhibiting at least one biological function leading to the PD-1/PD-L1 signal transduction pathway. The “mitigate”, “interfere”, “inhibit” or “block” the PD-1/PD-L1 inhibitory signal transduction pathways after specific binding of the Fc-CD80 fusion protein or its conjugate of the present invention to PD-L1 are not necessarily complete “mitigate”, “interfere”, “inhibit” or “block”.

The terms “CD28/B7 signal transduction pathway”, “CD28/B7 co-stimulatory pathway”, and “CD28/B7 pathway” used herein are used interchangeably herein and refer to (i) a signal transduction pathway that stimulates cell activation by binding of CD28 to CD80; and/or (ii) a signal transduction pathway that stimulates cell activation by binding CD28 to CD86.

“CD80” and “CD86” are both transmembrane glycoproteins, which are members of the immunoglobulin superfamily (IgSF) with a highly similar structure, and are also collectively referred as B7 molecules. Extracellular regions of CD80 and CD86 are each composed of an immunoglobulin V (IgV) region and an immunoglobulin C (IgC) region. A mature CD80 molecule is composed of 254 amino acids, of which 208 amino acids are in the extracellular region, 25 amino acids in the transmembrane region and 21 amino acids in the intracellular region. Similarly, a mature CD86 molecule is composed of 303 amino acids, of which 222 amino acids are in the extracellular region, 20 amino acids in the transmembrane region and 61 amino acids in the intracellular region.

CD80, also referred to as B7.1, is expressed on the surfaces of T cells, B cells, dendritic cells, and monocytes, and binds to CD28, PD-L1, and CTLA-4 at a relatively low affinity through its immunoglobulin V (IgV) region, wherein the binding affinity of CD80 to CD28 is 4 μM; the binding affinity of CD80 to PD-L1 is about 1.7 μM; and the binding affinity of CD80 to CTLA-4 is 0.2 μM (Butte M J, et al., Programmed death-1 ligand 1 interacts specifically with the B7-1 costimulatory molecule to inhibit T cell responses, Immunity, July 2007; 27(1): 111-122). CD86 binds to CD28 and CTLA-4, but not to PD-L1.

Soluble CD80 (e.g., CD80-Fc) is able to have a sustained activation effect on T lymphocytes through a CD28/B7 co-stimulatory pathway and stimulate the production of interferon. Experiments have shown that CD80-Fc maintains the production of interferon from T lymphocytes in vitro, and is even more effective than an anti-PD-1 antibody or an anti-PD-L1 antibody. In terms of in-vivo inhibition of tumor growth, soluble CD80 (e.g., CD80-Fc) is more effective than the anti-PD-L1 antibody (Ostrand-Rosenberg S, et al., Novel strategies for inhibiting PD-1 pathway-mediated immune suppression while simultaneously delivering activating signals to tumor-reactive T cells, Cancer Immunol Immunother, October 2015; 64(10): 1287-93). CD80-Fc can inhibit PD-1/PD-L1 pathway-mediated immunosuppression by binding to PD-L1 and deliver co-stimulatory signals to T cells activated by the CD28/B7 co-stimulatory pathway, thereby enhancing T lymphocyte activation. In summary, CD80-Fc can mitigate an immunosuppressive effect of the PD-1/PD-L1 pathway while activating tumor immunoreactive T cells. Although soluble CD86 (e.g., CD86-Fc) can also activate CD28 and even produce a 3-5-fold activation effect on CD80-Fc, eventually CD80-Fc has a stronger activation effect on T lymphocytes than CD86-Fc because CD86 does not bind to PD-L1 (Haile S T, et al., Soluble CD80 restores T cell activation and overcomes tumor cell programmed death ligand 1-mediated immune suppression, J Immunol., Sep. 1, 2013; 191(5): 2829-36).

Existing studies have shown that CD80-Fc has the following effects: (i) CD80-Fc has a better tumor inhibition effect than the PD-L1 antibody when used alone (AACR ANNUAL MEETING, Apr. 14-18, 2018, Chicago, Ill., USA); (ii) CD80-Fc promotes the infiltration of lymphocytes to a tumor tissue and has a better effect than the PD-L1 antibody (Horn L A, et al., Soluble CD80 Protein Delays Tumor Growth and Promotes Tumor-Infiltrating Lymphocytes, Cancer Immunol Res., January 2018; 6(1): 59-68); and (iii) CD80-Fc has a better tumor inhibition effect than inhibitors of the PD-1/PD-L1 pathway when used alone, and has a synergistic effect when combined with PD-1 antibodies. Five Prime Therapeutics, Inc. even considers CD80-Fc is superior to T cell agonists like GITRL, OX40L, and 4-1BBL. As a result of seeing the positive immunotherapy effect of CD80-Fc, Five Prime Therapeutics, Inc.'s CD80-Fc program, FPT155, plans to conduct clinical trials in the near future.

The terms “B7/CTLA-4 pathway” and “B7/CTLA-4 signal transduction pathway” are used interchangeably herein and refer to (i) a signal transduction pathway induced by the binding of CD80 to CTLA-4; and/or (ii) a signal transduction pathway induced by the binding of CD86 to CTLA-4.

Glypican-3 (GPC3) is a membranous heparan sulfate glycoprotein. The GPC3 protein is linked to a core protein by a heparan sulfate glucosamine glycan chain, and a carboxyl terminal of the core protein is anchored to the cell membrane surface by GPI. GPC3 is closely related to the occurrence and progression of liver cancer, melanoma and ovarian clear cell cancer. GPC3 expression has high specificity. It is highly expressed in liver cancer, slightly expressed in tumors such as melanoma, ovarian clear cell cancer, yolk sac tumor, neuroblastoma, hepatoblastoma and Wilm sarcoma cells, unexpressed in breast cancer, mesothelioma, ovarian epithelial cancer and lung cancer, and almost not expressed in normal human tissues, and thus expected to become one of the ideal targets for liver cancer immunotherapy (Ruan Jian, et al., Expression of Glypican-3 in malignant tumors and its clinical application, Oncology, 2011, 31(9): 863-866). At present, a total of four GPC3 antibodies have entered different research phases. GC33 (Ishiguro T., et al., Anti-glypican 3 antibody as a potential anti-tumor agent for human liver cancer. Cancer Res. 2008; 68(23): 9832-9838. Nakano K., et al., Generation of a humanized anti-glypican 3 antibody by CDR grafting and stability optimization. Anticancer Drugs. 2010; 21(10): 907-916) is the first humanized antibody to enter clinical research. GC33 is an antibody obtained by humanizing a mouse maternal antibody. GC33 recognizes polypeptide epitopes of carboxyl terminals (542-563) of Glypican-3, and exerts anti-tumor effects mainly through antibody-dependent cytotoxicity (ADCC) and recruitment of tumor-infiltrating lymphocytes (TIL). A Phase I clinical study (NCT00976170) in combination with sorafenib has been completed at present, and patients in a phase II clinical study are being recruited (NCT01507168). YP7 is a high-affinity humanized antibody (Phung Y, Gao W, Man Y G, Nagata S, Ho M., High-affinity monoclonal antibodies to cell surface tumor antigen glypican-3 generated through a combination of peptide immunization and flow cytometry screening. MAbs. September-October 2012; 4(5): 592-9). YP7 has an affinity K_{D} of 0.3 nM for Glypican-3, and recognizes polypeptide epitopes of carboxyl terminals (510-560) of Glypican-3. YP7 has a strong tumor suppressive activity. HN3 is a humanized single-domain antibody (Feng M. et al., Therapeutically targeting glypican-3 via a conformation-specific single-domain antibody in hepatocellular carcinoma. Proc Natl Acad Sci USA. 2013; 110(12): E1083-1091). This antibody can directly inhibit the proliferation of HCC cells. This antibody is screened by a phage antibody library technology. HN3 binds to a core protein site of Glypican-3 at high affinity. HN3 has a good inhibitory effect on positive liver cancer cells in vivo and in vitro. The uniqueness of HN3 lies in that it can directly inhibit the proliferation of tumor cells, participate in a YAP signaling pathway, and leads to cell cycle arrest. MDX-1414 is a fully-humanized antibody (Feng M, Ho M., Glypican-3 antibodies: a new therapeutic target for liver cancer. FEBS Lett. 2014 Jan. 21; 588(2): 377-82). MDX-1414 is screened by Medarex from multiple strains of fully-humanized antibodies, and has high affinity, strong specificity, internalization characteristics. In-vivo and in-vitro studies have shown that MDX-1414 has a better inhibition effect on tumor cell growth without any obvious toxic and side effects. It is still in the preclinical research phase.

The terms “VEGF/VEGFR pathway” and “VEGF/VEGFR signal transduction pathway” are used interchangeably herein and refer to one or more signal transduction pathways mediated by binding one or more members of VEGF family to one or more members of cell surface receptor VEGFR family. The VEGF family contains six closely related polypeptides, which are highly conserved homodimeric glycoproteins with six isoforms: VEGF-A, -B, -C, -D, -E, and a placental growth factor (PLGF), with molecular weights ranging from 35 kDa to 44 kDa. The expression of VEGF-A (including its splicing such as VEGF₁₆₅) is correlated with the microvascular density of some solid tumors, and the concentration of VEGF-A in tissues is associated with the prognosis of solid tumors such as breast cancer, lung cancer, prostate cancer, and colon cancer. The biological activity of each VEGF family member is mediated by one or more members of cell surface VEGF receptor (VEGFR) family. The VEGFR family includes VEGFR1 (also referred to as Flt-1), VEGFR2 (also referred to as KDR, Flk-1), VEGFR3 (also referred to as Flt-4), etc. Among them, VEGFR1 and VEGFR2 are closely related to angiogenesis, and VEGF-C/D/VEGFR3 is closely related to lymphangiogenesis. The main biological functions of the VEGF family include: (1) selectively promoting the mitosis of vascular endothelial cells, stimulating the endothelial cell proliferation and promoting the angiogenesis; (2) improving the permeability of blood vessels, especially small blood vessels, so that plasma macromolecules are extravasated and deposited in an extravascular matrix, thereby providing nutrients for the growth of tumor cells and the establishment of new capillary networks; (3) promoting the proliferation and metastasis of a tumor, wherein the proliferation and metastasis of the tumor rely on the VEGF family to make vascular endothelial cells secrete collagenase and plasminogen, thereby degrading a vascular basement membrane, and at the same time, the newly formed microvascular basement membrane inside the tumor tissues is imperfect, which makes the tumor easy to enter the blood circulation; (4) VEGF, as an immunosuppressive molecule, inhibiting the body's immune response and promoting the infiltration and metastasis of malignant tumors (Lapeyre-Prost A, et al., Immunomodulatory Activity of VEGF in Cancer, Int Rev Cell Mol Biol., 2017; 330: 295-342); (5) other functions: the VEGF families can induce gaps and fenestrations in epithelial cells, and can activate cytoplasmic vesicles and organelles of epithelial cells; the VEGF families directly stimulate endothelial cells to release proteolytic enzymes, degrade the matrix, release more VEGF family molecules and accelerate the development of tumors, and extracellular proteases in turn can activate the binding property of the extracellular matrix and the release of the VEGF family; the VEGF family releases plasma proteins (including fibrinogen) by increasing the vascular permeability to form a cellulose network, which provides a good matrix for tumor growth, development and metastasis; and the VEGF family promotes the production of abnormal blood vessels, and hinder the infiltration of immune cells. Clinical studies have shown that an anti-VEGF monoclonal antibody, an anti-VEGFR monoclonal antibody, or soluble VEGFR can be used to block the binding of the VEGF family to its receptors and hinder the transduction of the VEGF family signaling pathways. Bevacizumab (trade name: Avastin), developed by Genentech, is a recombinant human-mouse chimeric anti-VEGF antibody that can play an anti-angiogenic role by blocking the binding of VEGF-A to VEGFR, making VEGFR inactive. Bevacizumab is currently used in the treatment of metastatic colorectal cancer, lung cancer, breast cancer, pancreatic cancer, kidney cancer, and the like. Aflibercept, developed by Sanofi-aventis and Regeneron, is a kind of VEGF-Trap, which is a fusion protein obtained by fusing a VEGFR1 extracellular second domain and a VEGFR2 extracellular third domain with a human IgG1 constant region, and this fusion protein can exert anti-tumor effects on some tumor patients by inhibiting the angiogenesis.

HER2 belongs to the same HER family as an epidermal growth factor receptor EGFR (also referred to as HER1) and is a type I transmembrane glycoprotein, with an extracellular region containing 632 amino acids, a transmembrane region composed of 22 highly hydrophobic amino acids and an intracellular region composed of 580 amino acids at the C-terminal. Tyrosine in the positions of 1139, 1196 and 1246 at an intracellular C-terminal is taken as tyrosine phosphorylation sites. Tumor patients with high HER2 expression are often insensitive to radiotherapy and chemotherapy, and are prone to tumor metastasis, resulting in poor prognosis. HER2 is overexpressed in a variety of tumor tissues, including breast cancer (25 to 30%), ovarian cancer (18 to 43%), non-small cell lung cancer (13 to 55%), prostate cancer (5 to 46%), gastric cancer (21 to 64%), head and neck tumors (16 to 50%) and other malignant tumors derived from epithelial cells, but lowly expressed or unexpressed in adult normal tissues, and thus becomes an ideal target molecule for tumor immunotherapy. Therapeutic antibodies trastuzumab and pertuzumab, and trastuzumab emtansine (T-DM1) antibody-coupled drugs (ADC), which take HER2 as targets, have been on the market for many years and have been approved by the FDA for the clinical treatment of breast cancer and gastric cancer, and have achieved good therapeutic effects. At present, there are still a variety of HER2-targeted drugs margetuximab, timigutuzumab, trastuzumab deruxtecan, RC48, zenocutuzumab and A166 under development.

Transforming growth factor β (TGF-β) superfamily signal transduction plays an important role in the regulation of cell growth, differentiation, and development in many biological systems. Transforming growth factors include activin, TGFβ, and BMP, which, once bound to corresponding receptors, phosphorylate intracellular signal transduction molecules Smads, thereby activating a signaling pathway. In the tumor microenvironment, TGF-β plays an important role in immunosuppression. TGF-β regulates the production and functions of many types of immune cells. It inhibits the production and functions of effector T cells and antigen-presenting dendritic cells (DC cells) by directly promoting the proliferation of Treg cells, inhibits the immune system, and is an important component of the tumor microenvironment. TGF-β creates an immunosuppressive tumor microenvironment (TME) to promote tumor progression and metastasis. The TGF-β projects currently under research include an M7824 (anti-PD-L1-TGF-βRII.) bifunctional immune fusion protein (Lan Y, et al., Enhanced preclinical anti-tumor activity of M7824, a bifunctional fusion protein simultaneously targeting PD-L1 and TGF-β, Sci. Transl. Med. Jan. 17, 2018; 10(424)), metelimumab, lerdelimumab, fresolimumab, etc. A soluble TGF-βRII receptor has a good inhibitory effect on tumor growth (Rowland-Goldsmith, et al., Soluble type II transforming growth factor-beta receptor attenuates expression of metastasis-associated genes and suppresses pancreatic cancer cell metastasis. Mol Cancer Ther. 2002; 1(3): 161-167).

Trop-2 is a monomeric transmembrane cell surface glycoprotein, which is localized on a chromosome 1p32 region, is intron-free, has an encoding product containing 323 amino acids, including a signal peptide of 26 amino acids, an extracellular region of 248 amino acids, a transmembrane region of 23 amino acids and a cytoplasmic tail region of 26 amino acids, has a relative molecular mass of about 35 000, and thus is considered as a cancer-associated antigen. The Trop2 gene can lead to the overexpression of cyclin D, cyclin E, CDK2 and CDK4 by activating an ErK1/2 signaling pathway, while reducing tumorigenesis caused by the expression of p27 and E-cadherin (Liu T, Liu Y, Bao X., et al., Overexpression of TROP2 predicts poor prognosis of patients with cervical cancer and promotes the proliferation and invasion of cervical cancer cells by regulating ERK signal transduction pathway[J]. PLoS One, 2013, 8(9): e75864). The expression of Trop-2 is related to the migration and invasion of various tumors. TROP-2 is highly expressed in a variety of epithelial-derived tumors and is an ideal target for the treatment of malignant tumors. At present, the antibody drugs under development include sacituzumab, sacituzumab govitecan, SKB264, etc.

“Affinity” or “binding affinity” refers to the intrinsic binding affinity that reflects the interaction between members of a binding pair. The affinity of a molecule X for its partner Y can generally be represented by a dissociation constant (K_{D}), which is a ratio of a dissociation rate constant to an association rate constant (k_{off} and kₒₙ, respectively). The affinity can be measured by common methods known in the art. One specific method used to measure the affinity is surface plasmon resonance (SPR).

The term “antibody” is used herein in the broadest sense and includes, but is not limited to, monoclonal antibodies, polyclonal antibodies, and multispecific antibodies (e.g., bispecific antibodies) so long as they exhibit the desired antigen binding activity. The antibody may be a complete antibody (e.g., having two full-length light chains and two full-length heavy chains) of any type and subtype (e.g., IgM, IgD, IgG1, IgG2, IgG3, IgG4, IgE, IgA1, and IgA2).

The terms “full antibody”, “full-length antibody”, “full antibody” and “complete antibody” are used interchangeably herein to refer to an antibody having a structure substantially similar to a structure of a natural antibody.

The term “antibody heavy chain” refers to the larger one of two types of polypeptide chains present in an antibody molecule in their naturally occurring conformation, which determines in normal circumstances the category to which the antibody belongs.

The term “antibody light chain” refers to the smaller one of two types of polypeptide chains present in an antibody molecule in their naturally occurring conformation. κ light chain and λ light chain refer to two main types of antibody light chains.

The terms “antibody fragment” and “antigen-binding fragment” are used interchangeably herein and are a part or segment of an antibody or antibody chain that has fewer amino acid residues than a complete or full antibody or antibody chain, which can bind to an antigen or compete with a complete antibody (i.e., a complete antibody from which the antigen-binding fragment originates) for binding to an antigen. An antigen binding fragment can be prepared by a recombinant DNA technology, or by enzymatic or chemical cleavage of a complete antibody. The antigen binding fragment includes, but is not limited to, Fab, Fab′, F(ab′)₂, Fv, or single-chain Fv. The Fab fragment is a monovalent fragment composed of V_{L}, V_{H}, C_{L} and CH1 domains, for example, the Fab fragment can be obtained by papain digestion of a complete antibody. In addition, digestion of a complete antibody by pepsin below a disulfide bond in a hinge region produces F(ab′)₂, which is a dimer of Fab′ and is a bivalent fragment. F(ab′)₂ can be reduced under neutral conditions by breaking the disulfide bond in the hinge region, thus converting the F(ab′)₂ dimer to a Fab′ monomer. The Fab′ monomer is basically a Fab fragment with a hinged region (for a more detailed description of other antibody fragments, see Fundamental Immunology, edited by W. E. Paul, Raven Press, N.Y. (1993)). The Fv fragment consists of V_{L} and V_{H} domains of a single arm of the antibody. Additionally, although the two domains of the Fv fragment, V_{L} and V_{H}, are encoded by separate genes, they can be linked using a recombinant method by a synthetic linker that can be generated by taking the two domains as a single protein chain in which V_{L} and V_{H} regions are paired to form a single-chain Fv. The antibody fragment can be obtained by a chemical method, a recombinant DNA method or a protease digestion method.

The term “immunoglobulin” refers to a protein with a structure that has a naturally occurring antibody. For example, IgG-type immunoglobulin is a heterotetrameric glycoprotein of approximately 150,000 Daltons composed of two light chains and two heavy chains joined by a disulfide bond. From an N-terminal to a C-terminal, each heavy chain of the immunoglobulin has a variable region (VH) (also referred to as a variable heavy chain domain or heavy chain variable domain), followed by three constant domains (CH1, CH2 and CH3) (also referred as heavy chain constant regions). Similarly, from the N-terminal to the C-terminal, each light chain of the immunoglobulin has a variable region (VL) (also referred to as a variable light chain domain or light chain variable domain), followed by a constant light chain (CL) domain (also referred as light chain constant region). The heavy chains of the immunoglobulin can be assigned to one of 5 categories called α(IgA), δ(IgD), ε(IgE), γ(IgG) or μ(IgM), some of which can be further divided into subclasses such as γ₁(IgG1), γ₂(IgG2), γ₃(IgG₃), γ₄(IgG₄), α₁(IgA₁) and α₂(IgA₂). The light chains of the immunoglobulin can be divided into one of two types, called κ and λ, based on amino acid sequences of their constant domains. The immunoglobulin consists essentially of two Fab molecules and one Fc domain linked by an immunoglobulin hinge region.

The term “Fc domain” or “Fc region” are used herein to define a C-terminal region of an immunoglobulin heavy chain that contains at least a portion of constant region. This term includes a native sequence Fc region and a variant Fc region. The native immunoglobulin “Fc domain” contains two or three constant domains, namely a CH2 domain, a CH3 domain, and an optional CH4 domain. For example, in a native antibody, the immunoglobulin Fc domain includes second and third constant domains (a CH2 domain and a CH3 domain) containing two heavy chains derived from IgG, IgA, and IgD-type antibodies; or second, third, and fourth constant domains (a CH2 domain, a CH3 domain, and a CH4 domain) containing two heavy chains derived from IgM and IgE-type antibodies. Unless otherwise noted herein, amino acid residues in the Fc region or heavy chain constant region are numbered according to an EU numbering system (also known as an EU index) described by Kabat, et al., in Sequence of Proteins of Immunological Interes, 5^{th} ed., Public Health Service, National Institutes of Health, Bethesda, Md., in 1991. In some embodiments, the immunoglobulin Fc domain of the present invention is a dimeric protein including a pair of such immunoglobulin constant region polypeptides, each of which contains a further downstream portion of a hinge region, as well as CH2 and CH3 domains. Such “Fc” may or may not contain an S—S interchain bridge in the hinge region.

“Human immunoglobulin” is an immunoglobulin that possesses an amino acid sequence corresponding to immunoglobulin produced by a human or human cell or is derived from a non-human source using a human immunoglobulin library or other sequences that encode human immunoglobulin.

“Identity percentage (%)” of an amino acid sequence means a percentage of amino acid residues in a candidate sequence that are identical to amino acid residues of a specific amino acid sequence shown in this specification after the candidate sequence is compared with the specific amino acid sequence shown in this specification and, if necessary, vacancies are introduced to achieve a maximum sequence identity percentage, without considering no conservative permutation as part of sequence identity.

The term “effective linking” means that various specified components are in a relationship that allows them to function in an expected way.

The term “N-terminal” refers to the last amino acid at the N-terminal, and the term “C-terminal” refers to the last amino acid at the C-terminal.

The term “fusion” refers to direct linking of two or more components by a peptide bond or effective linking with the help of one or more peptide linkers. In some embodiments, the fusion protein of the present invention is a fusion protein that links the immunoglobulin Fc domain with a CD80 extracellular domain (ECD) directly by a peptide bond or effectively by means of one or more peptide linkers.

As used herein, the term “conjugate” refers to a polypeptide molecule including at least two components, wherein the first component includes an Fc-CD80 fusion protein, the second component includes a second effector molecule, and the first and second components are directly linked to each other by a peptide bond or by means of a peptide linker. The secondary effector molecule is any molecule that is capable of producing a beneficial biological effect except CD80, including but not limited to an antibody fragment, a receptor extracellular domain, cytotoxin, cytokine, a detectable label, a radioisotope, a therapeutic, a binding protein, or a molecule having a second amino acid sequence.

As used herein, the term “Fab-like fragment” is a second component of the conjugate of the present invention, which is formed such that the second effector molecule is linked to an N-terminal of a CH1 domain of an immunoglobulin heavy chain and a second effector molecule is linked to an N-terminal of a CL domain of an immunoglobulin light chain.

The term “host cells” refers to cells into which exogenous polynucleotides have been introduced, including progenies of such cells. The host cells include “transformants” and “transformed cells” which include primary transformed cells and progenies derived therefrom. The host cells are of any type of cell system that may be used to produce the Fc-CD80 fusion protein or its conjugate of the present invention. Host cells include cultured cells, as well as cells inside transgenic animals, transgenic plants, or cultured plant tissues or animal tissues.

The term “individual” or “subject” is used interchangeably to refer to mammals. Mammals include, but are not limited to, domesticated animals (e.g., cows, sheep, cats, dogs, and horses), primates (e.g., human and nonhuman primates such as monkeys), rabbits, and rodents (e.g., mice and rats). In particular, the individual is human beings.

The term “treatment” refers to a clinical intervention intended to change the natural course of a disease in an individual being treated. The desired therapeutic effects include, but are not limited to, preventing the onset or recurrence of the disease, alleviating symptoms, reducing any direct or indirect pathological consequences of the disease, preventing metastasis, reducing a progression rate of the disease, improving or alleviating a disease status, and alleviating or improving the prognosis. In some embodiments, the Fc-CD80 fusion protein of the present invention, the conjugate thereof or a pharmaceutical composition containing the Fc-CD80 fusion protein of the present invention and/or conjugate thereof are used to delay the development of a disease or to slow down the progression of the disease.

The term “anti-tumor effect” refers to a biological effect that can be exhibited by a variety of means including, but not limited to, for example, reduction in tumor volume, reduction in tumor cell number, reduction in tumor cell proliferation, or reduction in tumor cell survival. The terms “tumor”, “cancer” and “cancerous disease” are used interchangeably herein and cover solid tumors and fluid tumors.

### II. Fc-CD80 Fusion Protein and its Conjugate

A CD80 extracellular domain contained in the Fc-CD80 fusion protein of the present invention has the ability to bind to CD28, CTLA-4 and PD-L1. In the Fc-CD80 fusion protein, the CD80 extracellular domain is optionally linked to the immunoglobulin Fc domain by a linking peptide, and is located at the C-terminal of the Fc domain. The CD80 extracellular domain is an extracellular full-length (IgV and IgCQ or CD80-IgV functional domain of CD8, or a functional fragment thereof. The specific amino acid sequences are shown in Table 1. The terms “extracellular structural domain”, “extracellular domain”, and “extracellular functional domain” are used interchangeably herein.

The inventors have unexpectedly found that the placement of the CD80 extracellular domain at the C-terminal of Fc helps to improve the binding ability to CD28, CTLA-4 and PD-L1.

TABLE 1
Sequences of CD80 extracellular domain

| | Functional | |
|---|---|---|
| SEQ ID NO: | domain | Amino acid sequence |
| SEQ ID NO: 1 | CD80-IgV | VIHVTKEVKEVATLSCGHNVSVEELA |
| | | QTRIYWQKEKKMVLTMMSGDMNIWPE |
| | | YKNRTIFDITNNLSIVILALRPSDEG |
| | | TYECVVLKYEKDAFKREHLAEVTLSV |
| | | KA |
| SEQ ID NO: 2 | CD80-IgV/C | VIHVTKEVKEVATLSCGHNVSVEELA |
| | | QTRIYWQKEKKMVLTMMSGDMNIWPE |
| | | YKNRTIFDITNNLSIVILALRPSDEG |
| | | TYECVVLKYEKDAFKREHLAEVTLSV |
| | | KADFPTPSISDFEIPTSNIRRIICST |
| | | SGGFPEPHLSWLENGEELNAINTTVS |
| | | QDPETELYAVSSKLDFNMTTNHSFMC |
| | | LIKYGHLRVNQTFNWNTTKQEHFPDN |

present invention further provides a conjugate including the Fc-CD80 fusion protein as a first component; and a second component containing a second effector molecule, the second effector molecule being, for example, an antibody fragment, a receptor extracellular domain or other proteins (e.g., cytokines). In one embodiment, the second component consists of a second effector molecule located at the N-terminal to the Fc-CD80 fusion protein. In another embodiment, the second component includes a second effector molecule. For example, the second component is a “Fab-like fragment”, which is formed such that the second effector molecule is linked to an N-terminal of a CH1 domain of an immunoglobulin heavy chain and a second effector molecule is linked to an N-terminal of a CL domain of an immunoglobulin light chain.

In the conjugate of the present invention, when the second component is a Fab fragment of an antibody, the Fab fragment forms an IgG molecule with Fc in the Fc-CD80 fusion protein. In some embodiments, IgG molecular categories include IgG1, IgG2 or IgG4. In one embodiment, IgG4 is mutated with S228P in an IgG4 constant region in order to prevent the occurrence of arm-exchange.

TABLE 2
Heavy-chain constant region sequence
of the IgG molecule

| Heavy chain | |
|---|---|
| categories | Amino acid sequence |
| IgG1 | TLVTVSSASTKGPSVFPLAPSSKSTSGGTAALGCLVKD |
| | YFPEPVTVSWNSGALTSGVHTFPAVLQSSGLYSLSSVV |
| | TVPSSSLGTQTYICNVNHKPSNTKVDKKVEPKSCDKTH |
| | TCPPCPAPELLGGPSVFLFPPKPKDTLMISRTPEVTCV |
| | VVDVSHEDPEVKFNWYVDGVEVHNAKTKPREEQYNSTY |
| | RVVSVLTVLHQDWLNGKEYKCKVSNKALPAPIEKTISK |
| | AKGQPREPQVYTLPPSREEMTKNQVSLTCLVKGFYPSD |
| | IAVEWESNGQPENNYKTTPPVLDSDGSFFLYSKLTVDK |
| | SRWQQGNVFSCSVMHEALHNHYTQKSLSLSPGK |
| | (SEQ ID NO: 3) |
| IgG2 | TTVTVSTASTKGPSVFPLAPCSRSTSESTAALGCLVKD |
| | YFPEPVTVSWNSGALTSGVHTFPAVLQSSGLYSLSSVV |
| | TVPSSNFGTQTYTCNVDHKPSNTKVDKTVERKCCVECP |
| | PCPAPPVAGPSVFLFPPKPKDTLMISRTPEVTCVVVDV |
| | SHEDPEVQFNWYVDGVEVHNAKTKPREEQFNSTFRVVS |
| | VLTVVHQDWLNGKEYKCKVSNKGLPAPIEKTISKTKGQ |
| | PREPQVYTLPPSREEMTKNQVSLTCLVKGFYPSDIAVE |
| | WESNGQPENNYKTTPPMLDSDGSFFLYSKLTVDKSRWQ |
| | QGNVFSCSVMHEALHNHYTQKSLSLSPGK |
| | (SEQ ID NO: 4) |
| IgG4 | TLVTVSSASTKGPSVFPLAPCSRSTSESTAALGCLVKD |
| | YFPEPVTVSWNSGALTSGVHTFPAVLQSSGLYSLSSVV |
| | TVPSSSLGTKTYTCNVDHKPSNTKVDKRVESKYGPPCP |
| | PCPAPEFLGGPSVFLFPPKPKDTLMISRTPEVTCVVVD |
| | VSQEDPEVQFNWYVDGVEVHNAKTKPREEQFNSTYRVV |
| | SVLTVLHQDWLNGKEYKCKVSNKGLPSSIEKTISKAKG |
| | QPREPQVYTLPPSQEEMTKNQVSLTCLVKGFYPSDIAV |
| | EWESNGQPENNYKTTPPVLDSDGSFFLYSRLTVDKSRW |
| | QEGNVFSCSVMHEALHNHYTQKSLSLSLGK |
| | (SEQ ID NO: 5) |

In some embodiments, a light-chain constant region type of the IgG molecule is a κ or λ type, preferably the K type.

TABLE 3
Light-chain constant region sequence

| Light-chain | |
|---|---|
| region type | Amino acid sequence |
| κ | TKVEIKRTVAAPSVFIFPPSDEQLKSGTASVVCLLNNF |
| | YPREAKVQWKVDNALQSGNSQESVTEQDSKDSTYSLSS |
| | TLTLSKADYEKHKVYACEVTHQGLSSPVTKSFNRGEC |
| | (SEQ ID NO: 6) |
| λ | TKLTVLGQPKAAPSVTLFPPSSEELQANKATLVCLISD |
| | FYPGAVTVAWKADSSPVKAGVETTTPSKQSNNKYAASS |
| | YLSLTPEQWKSHRSYSCQVTHEGSTVEKTVAPTECS |
| | (SEQ ID NO: 7) |

In some embodiments, Fc of the IgG molecule includes CH2 and CH3 of IgG1, IgG2 or IgG4.

TABLE 4
Human Fc molecule (CH2—CH3) sequences

| Heavy chain | |
|---|---|
| categories | Fc region amino acid sequence |
| IgG1 | DKTHTCPPCPAPELLGGPSVFLFPPKPKDTLMISRTPE |
| | VTCVVVDVSHEDPEVKFNWYVDGVEVHNAKTKPREEQY |
| | NSTYRVVSVLTVLHQDWLNGKEYKCKVSNKALPAPIEK |
| | TISKAKGQPREPQVYTLPPSREEMTKNQVSLTCLVKGF |
| | YPSDIAVEWESNGQPENNYKTTPPVLDSDGSFFLYSKL |
| | TVDKSRWQQGNVFSCSVMHEALHNHYTQKSLSLSPGK |
| | (SEQ ID NO: 8) |
| IgG2 | ERKCCVECPPCPAPPVAGPSVFLFPPKPKDTLMISRTP |
| | EVTCVVVDVSHEDPEVQFNWYVDGVEVHNAKTKPREEQ |
| | FNSTFRVVSVLTVVHQDWLNGKEYKCKVSNKGLPAPIE |
| | KTISKTKGQPREPQVYTLPPSREEMTKNQVSLTCLVKG |
| | FYPSDIAVEWESNGQPENNYKTTPPMLDSDGSFFLYSK |
| | LTVDKSRWQQGNVFSCSVMHEALHNHYTQKSLSLSPGK |
| | (SEQ ID NO: 9) |
| IgG4 | ESKYGPPCPPCPAPEFLGGPSVFLFPPKPKDTLMISRT |
| | PEVTCVVVDVSQEDPEVQFNWYVDGVEVHNAKTKPREE |
| | QFNSTYRVVSVLTVLHQDWLNGKEYKCKVSNKGLPSSI |
| | EKTISKAKGQPREPQVYTLPPSQEEMTKNQVSLTCLVK |
| | GFYPSDIAVEWESNGQPENNYKTTPPVLDSDGSFFLYS |
| | RLTVDKSRWQEGNVFSCSVMHEALHNHYTQKSLSLSLG |
| | K |
| | (SEQ ID NO: 10) |

In some embodiments, an amino acid sequence of a linking peptide may be selected from, but not limited to, any of the following sequences: AKTTPKLEEGEFSEAR(SEQ ID NO: 11), AKTTPKLEEGEFSEARV(SEQ ID NO: 12); AKTTPKLGG(SEQ ID NO: 13); SAKTTPKLGG(SEQ ID NO: 14); SAKTTP(SEQ ID NO: 15); RADAAP(SEQ ID NO: 16), RADAAPTVS(SEQ ID NO: 17); RADAAAAGGPGS(SEQ ID NO: 18), RADAAAA(SEQ ID NO: 19), SAKTTPKLEEGEFSEARV(SEQ ID NO:20); ADAAP(SEQ ID NO:21), DAAPTVSIFPP(SEQ ID NO:22), TVAAP(SEQ ID NO:23); TVAAPSVFIFPP(SEQ ID NO:24); QPKAAP(SEQ ID NO:25), QPKAAPSVTLFPP(SEQ ID NO:26); AKTTPP(SEQ ID NO:27), AKTTPPSVTPLAP(SEQ ID NO:28), AKTTAP(SEQ ID NO:29); AKTTAPSVYPLAP(SEQ ID NO:30); ASTKGP(SEQ ID NO:31), ASTKGPSVFPLAP(SEQ ID NO:32); GGGGSGGGGSGGGGS(SEQ ID NO:33), GENKVEYAPALMALS(SEQ ID NO:34); GPAKELTPLKEAKVS(SEQ ID NO:35); GHEAAAVMQVQYPAS(SEQ ID NO:36). In some embodiments, the linking peptide is (G4S)n, wherein n is an integer of 1-5, and preferably the linking peptide is GGGGSGGGGSGGGGS (SEQ ID NO: 33).

In the conjugate of the present invention, the second effector molecule in the second component may be an antibody fragment, a receptor extracellular domain or other proteins (e.g., cytokines).

In some embodiments, the second effector molecule is such an antibody fragment that specifically binds to a tumor-specific antigen or a tumor-associated antigen. The tumor-specific antigen or tumor-associated antigen includes, but is not limited to: an epidermal growth factor receptor (EGFR1), HER2/neu, CD20, a vascular endothelial growth factor (VEGF), an insulin-like growth factor receptor (IGF-1R), a TRAIL receptor, an epithelial cell adhesion molecule, a carcinoembryonic antigen, a prostate specific membrane antigen (PSMA), Mucin-1, CD30, CD33, Trop-2, CD40, CD137, Ang2, cMet; PDGF, DLL-4; CD138, CD19, CD133; CD38, CD22, ErbB3, angiopoietin-2 (Ang-2), TWEAK, CLDN18.2, CD73, MSTN (myostatin, growth differentiation factor 8), AXL (AXL receptor tyrosine kinase), TNFRSF12A (Tumor Necrosis Factor Receptor (TNFR) Superfamily, member 12A), PVRL4 (4 associated with poliovirus receptor 4), MUC5AC (mucin 5AC), ITGAV_ITGB3 (Integrin αV_β3), FOLR1 (Folate Receptor 1), GPNMB (Glycoprotein (transmembrane) nmb), SDC1 (Syndecan-1), ENG (Endoglin), CA9 (Carbonic Anhydrase IX), PTPRC (Protein Tyrosine Phosphatase Receptor Type C), a DNA/histone (H1) complex, CEACAM5 (Carcinoembryonic Antigen-related Cell Adhesion Molecule 5), SLC39A6 (Solute Carrier Family 39, member 6), TNFRSF10B (Tumor Necrosis Factor Receptor (TNFR) Superfamily, member 10B), SLC34A2 (Solute Carrier Family 34 Sodium Phosphate, member 2), KIRD2 subgroup (Killer Cell Immunoglobulin-like Receptor from KIRD2 Subgroup), TNFRSF10A (Tumor Necrosis Factor Receptor (TNFR) Superfamily, member 10A), ganglioside GD3, CCR4 (Chemokine (C-C motif) Receptor 4), KLRC1 (Killer Cell Lectin-like Receptor Superfamily C, member 1), AMHR2 (Anti-Mullerian Hormone Receptor type 2), PDGFRA (Platelet-derived Growth Factor Receptor a Subunit), CD248 (Endothelin, Tumor Endothelial Marker 1), EGFL7 (Epidermal Growth Factor (EGF)-like Repeat Superfamily, member 7), CD79B (Immunoglobulin-associated CD79β)), ALCAM (Activated Leukocyte Adhesion Molecule CD166), VIM (Vimentin), MAG (Myelin-Associated Glycoprotein), PRLR (Prolactin Receptor), DLL3 (6-like 3), CD200 (OX-2), LRRC15 (Leucine-rich Repeat-containing Protein 15), SLITRK6 (SLIT and NTRK-like Family, member 6), FZD10 (Frizzled Class Receptor 10), NOTCH2 (Notch Protein 2), NOTCH3 (Notch Protein 3), EPCAM (Epithelial Cell Adhesion Molecule, tumor-associated calcium signaling transducer 1), ITGAV (Integrin αV), ACVRL1 (Activin A Receptor Type II Like Protein 1), CSF1R (Colony-stimulating Factor 1 Receptor), ACVR2A (Activin A Receptor Type IIA), MUC1 sialylated carbohydrate tumor-associated (CA242), ganglioside GD2, EPHA3 (Ephrin Receptor A3), GUCY2C (Guanylate Cyclase 2C), PTPRC (Protein Tyrosine Phosphatase Receptor Type C), and IL1RAP (Interleukin 1 Receptor Accessory Protein).

The antibody fragment includes, but is not limited to, those derived from: cetuximab, trastuzumab, abciximab, daclizumab, basiliximab, palivizumab, infliximab, gemtuzumab ozogamicin, alemtuzumab, ibritumomab tiuxetan, adalimumab, omalizumab, tositumomab, efalizumab, bevacizumab, panitumumab, natalizumab, IGN101 (Aphton), volociximab (Biogen Idec and PDL BioPharm), anti-CD23 mAb (Biogen Idel), CAT-3888 (Cambridge Antibody Technology), sacituzumab, CDP-791 (Imclone), eraptuzumab (Immunomedics), MDX-010 (Medarex and BMS), MDX-060 (Medarex), MDX-070 (Medarex), matuzumab (Merck), CP-675, 206 (Pfizer), CAL (Roche), SGN-30 (Seattle Genetics), zanolimumab (Serono and Genmab), adecatumumab (Sereno), oregovomab (United Therapeutics), nimotuzumab (YM Bioscience), ABT-874 (Abbott Laboratories), denosumab (Amgen), AM 108 (Amgen), AMG 714 (Amgen), fontolizumab (Biogen Idec and PDL BioPharm), daclizumab (Biogent Idec and PDL BioPharm), golimumab (Centocor and Schering-Plough), CNTO 1275 (Centocor), ocrelizumab (Genetech and Roche), HuMax-CD20 (Genmab), belimumab (HGS and GSK), epratuzumab (Immunomedics), MLN1202 (Millennium Pharmaceuticals), visilizumab (PDL BioPharm), tocilizumab (Roche), ocrerlizumab (Roche), certolizumab pegol (UCB, previously, a Celltech), eculizumab (Alexion Pharmaceuticals), pexelizumab (Alexion Pharmaceuticals and Procter & Gamble), abciximab (Centocor), raIbizimumab (Genetech), mepolizumab (GSK), TNX-355 (Tanox), capromab, oleclumab, NZV930, TJD5, domagrozumab, enapotamab, enavatuzumab, enfortumab vedotin, ensituximab, clivatuzumab tetraxetan, cantuzumab mertansine, intetumumab, etaracizumab, mirvetuximab, farletuzumab, glembatumumab, indatuximab ravtansine, carotuximab, derlotuximab biotin, altumomab, labetuzumab, ladiratuzumab, benufutamab, conatumumab, tigatuzumab, drozitumab, lexatumumab, lifastuzumab vedotin, lirilumab, mapatumumab, ecromeximab, mitumomab, mogamulizumab, monalizumab, murlentamab, olaratumab, tovetumab, ontuxizumab, parsatuzumab, iladatuzumab, polatuzumab, praluzatamab, pritumumab, refanezumab, rolinsatamab, rovalpituzumab, samalizumab, samrotamab, samrotamab vedotin, sirtratumab, sirtratumab vedotin, tabituximab, tabituximab barzuxetan, brontictuzumab, tarextumab, adecatumumab, catumaxomab, edrecolomab, labetuzumab, demcizumab, dilpacimab, enoticumab, navicixizumab, Faricimab, nesvacumab, vanucizumab, hCTM01, abituzumab, ascrinvacumab, axatilimab, cabiralizumab, emactuzumab, bimagrumab, dinutuximab, ifabotuzumab, indusatumab, apamistamab, nidanilimab, or MYO-029 (Wyeth). In some embodiments, the antibody fragment is, for example, Fab, Fab′, F(ab′)₂, Fv, or single-strain Fv.

Table 5 exemplifies tumor-specific antigens or tumor-associated antigens that are regarded as the targets of the second effector molecules in the conjugate, names of the second effector molecules (e.g., antibodies), and variable region amino acid sequences of the second effector molecules (e.g., antibodies).

TABLE 5
Tumor-specific antigens or tumor-associated antigen targets,
names of antibodies, and variable region
amino acid sequences of the antibodies

| | | INN | light chain or | Variable region amino acid |
|---|---|---|---|---|
| Target | Antibody | Num. | heavy chain | sequence of antibodies |
| VEGF | Bevacizumab | 8017 | H | EVQLVESGGGLVQPGGSLRLSCAASGYTFTNYGMN |
| | | | | WVRQAPGKGLEWVGWINTYTGEPTYAADFKRRFTF |
| | | | | SLDTSKSTAYLQMNSLRAEDTAVYYCAKYPHYYGS |
| | | | | SHWYFDVWGQGTLVTV |
| | | | | (SEQ ID NO: 37) |
| | | | L | DIQMTQSPSSLSASVGDRVTITCSASQDISNYLNW |
| | | | | YQQKPGKAPKVLIYFTSSLHSGVPSRFSGSGSGTD |
| | | | | FTLTISSLQPEDFATYYCQQYSTVPWTFGQG |
| | | | | (SEQ ID NO: 38) |
| HER2 | Trastuzumab | 7637 | H | EVQLVESGGGLVQPGGSLRLSCAASGFNIKDTYIH |
| | | | | WVRQAPGKGLEWVARIYPTNGYTRYADSVKGRFTI |
| | | | | SADTSKNTAYLQMNSLRAEDTAVYYCSRWGGDGFY |
| | | | | AMDYWGQG (SEQ ID NO: 39) |
| | | | L | DIQMTQSPSSLSASVGDRVTITCRASQDVNTAVAW |
| | | | | YQQKPGKAPKLLIYSASFLYSGVPSRFSGSRSGTD |
| | | | | FTLTISSLQPEDFATYYCQQHYTTPPTFGQG |
| | | | | (SEQ ID NO: 40) |
| EGFR | Cetuximab | 7906 | H | QVQLKQSGPGLVQPSQSLSITCTVSGFSLTNYGVH |
| | | | | WVRQSPGKGLEWLGVIWSGGNTDYNTPFTSRLSIN |
| | | | | KDNSKSQVFFKMNSLQSNDTAIYYCARALTYYDYE |
| | | | | FAYWGQG (SEQ ID NO: 41) |
| | | | L | DILLTQSPVILSVSPGERVSFSCRASQSIGTNIHW |
| | | | | YQQRTNGSPRLLIKYASESISGIPSRFSGSGSGTD |
| | | | | FTLSINSVESEDIADYYCQQNNNWPTTFGAG |
| | | | | (SEQ ID NO: 42) |
| CD20 | Rituximab | 7609 | H | QVQLQQPGAELVKPGASVKMSCKASGYTFTSYNMH |
| | | | | WVKQTPGRGLEWIGAIYPGNGDTSYNQKFKGKATL |
| | | | | TADKSSSTAYMQLSSLTSEDSAVYYCARSTYYGGD |
| | | | | WYFNVWGAG (SEQ ID NO: 43) |
| | | | L | QIVLSQSPAILSASPGEKVTMTCRASSSVSYIHWF |
| | | | | QQKPGSSPKPWIYATSNLASGVPVRFSGSGSGTSY |
| | | | | SLTISRVEAEDAATYYCQQWTSNPPTFGGG |
| | | | | (SEQ ID NO: 44) |
| Phosphat- | Codrituzumab | 9759 | H | QVQLVQSGAEVKKPGASVKVSCKASGYTFTDYEMH |
| idylinositol | | | | WVRQAPGQGLEWMGALDPKTGDTAYSQKFKGRVTL |
| proteoglycan 3 | | | | TADKSTSTAYMELSSLTSEDTAVYYCTRFYSYTYW |
| | | | | GQG (SEQ ID NO: 45) |
| | | | L | DVVMTQSPLSLPVTPGEPASISCRSSQSEVHSNRN |
| | | | | TYLHWYLQKPGQSPQLLIYKVSNRFSGVPDRFSGS |
| | | | | GSGTDFTLKISRVEAEDVGVYYCSQNTHVPPTFGQ |
| | | | | G (SEQ ID NO: 46) |
| CTLA-4 | Ipilimumab | 8568 | H | QVQLVESGGGVVQPGRSLRLSCAASGFTFSSYTMH |
| | | | | WVRQAPGKGLEWVTFISYDGNNKYYADSVKGRFTI |
| | | | | SRDNSKNTLYLQMNSLRAEDTAIYYCARTGWLGPF |
| | | | | DYWGQG (SEQ ID NO: 47) |
| | | | L | EIVLTQSPGTLSLSPGERATLSCRASQSVGSSYLA |
| | | | | WYQQKPGQAPRLLIYGAFSRATGIPDRFSGSGSGT |
| | | | | DFTLTISRLEPEDFAVYYCQQYGSSPWTFGQG |
| | | | | (SEQ ID NO: 48) |
| CD30 | Iratumumab | 8713 | H | QVQLQQWGAGLLKPSETLSLTCAVYGGSFSAYYWS |
| | | | | WIRQPPGKGLEWIGDINHGGGTNYNPSLKSRVTIS |
| | | | | VDTSKNQFSLKLNSVTAADTAVYYCASLTAYWGQG |
| | | | | (SEQ ID NO: 49) |
| | | | L | DIQMTQSPTSLSASVGDRVTITCRASQGISSWLTW |
| | | | | YQQKPEKAPKSLIYAASSLQSGVPSRFSGSGSGTD |
| | | | | FTLTISSLQPEDFATYYCQQYDSYPITFGQG |
| | | | | (SEQ ID NO: 50) |
| CD33 | Gemtuzumab | 8024 | H | EVQLVQSGAEVKKPGSSVKVSCKASGYTITDSNIH |
| | | | | WVRQAPGQSLEWIGYIYPYNGGTDYNQKFKNRATL |
| | | | | TVDNPTNTAYMELSSLRSEDTAFYYCVNGNPWLAY |
| | | | | WGQG (SEQ ID NO: 51) |
| | | | L | DIQLTQSPSTLSASVGDRVTITCRASESLDNYGIR |
| | | | | FLTWFQQKPGKAPKLLMYAASNQGSGVPSRFSGSG |
| | | | | SGTEFTLTISSLQPDDFATYYCQQTKEVPWSFGQG |
| | | | | (SEQ ID NO: 52) |
| CEA | Cergutuzumab | 10080 | H | QVQLVQSGAEVKKPGASVKVSCKASGYTFTEFGMN |
| | | | | WVRQAPGQGLEWMGWINTKTGEATYVEEFKGRVTF |
| | | | | TTDTSTSTAYMELRSLRSDDTAVYYCARWDFAYYV |
| | | | | EAMDYWGQG (SEQ ID NO: 53) |
| | | | L | DIQMTQSPSSLSASVGDRVTITCKASAAVGTYVAW |
| | | | | YQQKPGKAPKLLIYSASYRKRGVPSRFSGSGSGTD |
| | | | | FTLTISSLQPEDFATYYCHQYYTYPLFTFGQG |
| | | | | (SEQ ID NO: 54) |
| IGF1R | Teprotumumab | 9107 | H | QVELVESGGGVVQPGRSQRLSCAASGFTFSSYGMH |
| | | | | WVRQAPGKGLEWVAIIWFDGSSTYYADSVRGRFTI |
| | | | | SRDNSKNTLYLQMNSLRAEDTAVYFCARELGRRYF |
| | | | | DLWGRG (SEQ ID NO: 55) |
| | | | L | EIVLTQSPATLSLSPGERATLSCRASQSVSSYLAW |
| | | | | YQQKPGQAPRLLIYDASKRATGIPARFSGSGSGTD |
| | | | | FTLTISSLEPEDFAVYYCQQRSKWPPWTFGQG |
| | | | | (SEQ ID NO: 56) |
| Trop-2 | Sacituzumab | 10418 | H | QVQLQQSGSELKKPGASVKVSCKASGYTFTNYGMN |
| | | | | WVKQAPGQGLKWMGWINTYTGEPTYTDDFKGRFAF |
| | | | | SLDTSVSTAYLQISSLKADDTAVYFCARGGFGSSY |
| | | | | WYFDVWGQG (SEQ ID NO: 57) |
| | | | L | DIQLTQSPSSLSASVGDRVSITCKASQDVSIAVAW |
| | | | | YQQKPGKAPKLLIYSASYRYTGVPDRFSGSGSGTD |
| | | | | FTLTISSLQPEDFAVYYCQQHYITPLTFGAG |
| | | | | (SEQ ID NO: 58) |

In some embodiments, the second effector molecule is such an antibody fragment, which can specifically bind to an immune checkpoint molecule of an immune cell and relieve an inhibitory effect on a tumor immune system. The immune checkpoint molecule includes, but is not limited to: PD1, PD-L1, CTLA-4, TIM-3, LAG-3, TIGIT, STING, VISTA, CD47, or Siglec-15 (S15) molecule.

The antibody fragments include, but are not limited to, those derived from nivolumab, pembrolizumab, camrelizumab, cemiplimab, pidilizumab, spartalizumab, atezolizumab, avelumab, durvalumab, ipilimumab, tremelimumab, cobolimab, relatlimab, tiragolumab, etigilimab, vibostolimab, magrolimab, NC318, REGN3767, LAG525, MTIG7192A, JNJ-61610588, TIM-3 (LY3321367, MBG453, MEDI9447, TSR-022), 189-192 LAG-3 (BMS-986016, LAG525), B7-H3 (enoblituzumab, 8H-9). In some embodiments, the antibody fragment is, for example, Fab, Fab′, F(ab′)₂, Fv, or single-strain Fv.

Table 6 exemplifies immune checkpoint molecules that are regarded as the targets of the second effector molecules in the conjugate, names of the second effector molecules (e.g., antibodies), and variable region amino acid sequences of the second effector molecules (e.g., antibodies).

TABLE 6
Targets of the immune checkpoint molecules, names of antibodies,
and variable region amino acid sequences of the antibodies

| | | INN | light chain or | Variable region amino acid |
|---|---|---|---|---|
| Target | Antibody | Num. | heavy chain | sequence of antibodies |
| PD1 | Nivolumab | 9623 | H | QVQLVESGGGVVQPGRSLRLDCKASGITFSNSGMH |
| | | | | WVRQAPGKGLEWVAVIWYDGSKRYYADSVKGRFTI |
| | | | | SRDNSKNTLFLQMNSLRAEDTAVYYCATNDDYWGQ |
| | | | | G (SEQ ID NO: 59) |
| | | | L | EIVLTQSPATLSLSPGERATLSCRASQSVSSYLAW |
| | | | | YQQKPGQAPRLLIYDASNRATGIPARFSGSGSGTD |
| | | | | FTLTISSLEPEDFAVYYCQQSSNWPRTFGQG |
| | | | | (SEQ ID NO: 60) |
| | Pembrolizumab | 9798 | H | QVQLVQSGVEVKKPGASVKVSCKASGYTFTNYYMY |
| | | | | WVRQAPGQGLEWMGGINPSNGGTNFNEKFKNRVTL |
| | | | | TTDSSTTTAYMELKSLQFDDTAVYYCARRDYRFDM |
| | | | | GFDYWGQG (SEQ ID NO: 61) |
| | | | L | EIVLTQSPATLSLSPGERATLSCRASKGVSTSGYS |
| | | | | YLHWYQQKPGQAPRLLIYLASYLESGVPARFSGSG |
| | | | | SGTDFTLTISSLEPEDFAVYYCQHSRDLPLTFGGG |
| | | | | (SEQ ID NO: 62) |
| PD-L1 | Atezolizumab | 9814 | H | EVQLVESGGGLVQPGGSLRLSCAASGFTFSDSWIH |
| | | | | WVRQAPGKGLEWVAWISPYGGSTYYADSVKGRFTI |
| | | | | SADTSKNTAYLQMNSLRAEDTAVYYCARRHWPGGF |
| | | | | DYWGQG (SEQ ID NO: 63) |
| | | | L | DIQMTQSPSSLSASVGDRVTITCRASQDVSTAVAW |
| | | | | YQQKPGKAPKLLIYSASFLYSGVPSRFSGSGSGTD |
| | | | | FTLTISSLQPEDFATYYCQQYLYHPATFGQG |
| | | | | (SEQ ID NO: 64) |
| CTLA-4 | Ipilimumab | 8568 | H | QVQLVESGGGVVQPGRSLRLSCAASGFTFSSYTMH |
| | | | | WVRQAPGKGLEWVTFISYDGNNKYYADSVKGRFTI |
| | | | | SRDNSKNTLYLQMNSLRAEDTAIYYCARTGWLGPF |
| | | | | DYWGQG (SEQ ID NO: 65) |
| | | | L | EIVLTQSPGTLSLSPGERATLSCRASQSVGSSYLA |
| | | | | WYQQKPGQAPRLLIYGAFSRATGIPDRFSGSGSGT |
| | | | | DFTLTISRLEPEDFAVYYCQQYGSSPWTFGQG |
| | | | | (SEQ ID NO: 66) |
| TIM-3 | Cobolimab | 10788 | H | EVQLLESGGGLVQPGGSLRLSCAAASGFTFSSYDM |
| | | | | SWVRQAPGKGLDWVSTISGGGTYTYYQDSVKGRFT |
| | | | | ISRDNSKNTLYLQMNSLRAEDTAVYYCASMDYWGQ |
| | | | | G (SEQ ID NO: 67) |
| | | | L | DIQMTQSPSSLSASVGDRVTITCRASQSIRRYLNW |
| | | | | YHQKPGKAPKLLIYGASTLQSGRFSGSGSGTDFTL |
| | | | | TISSLQPEDFAVYYCQQSHSAPLTFGGG |
| | | | | (SEQ ID NO: 68) |
| LAG-3 | Relatlimab | 10735 | H | QVQLQQWGAGLLKPSETLSLTCAVYGGSFSDYYWN |
| | | | | WIRQPPGKGLEWIGEINHRGSTNSNPSLKSRVTLS |
| | | | | LDTSKNQFSLKLRSVTAADTAVYYCAFGYSDYEYN |
| | | | | WFDPWGQG (SEQ ID NO: 69) |
| | | | L | EIVLTQSPATLSLSPGERATLSCRASQSISSYLAW |
| | | | | YQQKPGQAPRLLIYDASNRATGIPARFSGSGSGTD |
| | | | | FTLTISSLEPEDFAVYYCQQRSNWPLTFGQG |
| | | | | (SEQ ID NO: 70) |
| TIGIT | Tiragolumab | 10644 | H | EVQLQQSGPGLVKPSQTLSLTCAISGDSVSSNSAA |
| | | | | WNWIRQSPSRGLEWLGKTYYRFKWYSDYAVSVKGR |
| | | | | ITINPDTSKNQFSLQLNSVTPEDTAVFYCTRESTT |
| | | | | YDLLAGPFDYWGQG (SEQ ID NO: 71) |
| | | | L | DIVMTQSPDSLAVSLGERATINCKSSQTVLYSSNN |
| | | | | KKYLAWYQQKPGQPPNLLIYWASTRESGVPDRFSG |
| | | | | SGSGTDFTLTISSLQAEDVAVYYCQQYYSTPFTFG |
| | | | | PG (SEQ ID NO: 72) |
| CD47 | Magrolimab | 10999 | H | QVQLVQSGAEVKKPGASVKVSCKASGYTFTNYNMH |
| | | | | WVRQAPGQRLEWMGTIYPGNDDTSYNQKFKDRVTI |
| | | | | TADTSASTAYMELSSLRSEDTAVYYCARGGYRAMD |
| | | | | YWGQG (SEQ ID NO: 73) |
| | | | L | DIVMTQSPLSLPVTPGEPASISCRSSQSIVYSNGN |
| | | | | TYLGWYLQKPGQSPQLLIYKVSNRFSGVPDRFSGS |
| | | | | GSGTDFTLKISRVEAEDVGVYYCFQGSHVPYTFGQ |
| | | | | G (SEQ ID NO: 74) |

In some embodiments, the second effector molecule is such an antibody fragment, which can specifically bind to an immune agonist molecule of an immune cell and enhance the immune response of the immune system to tumors. The immune agonist molecule includes, but is not limited to: GITR, 4-1BBL, OX40, ICOS, TLR2 or CD27 and other molecules. The antibody fragments include, but are not limited to those derived from TRX518, AMG 228, urelumab, utomilumab, ivuxolimab, oxelumab, tavolimab, vonlerolizumab, varlilumab, GITR (TRX-518, BMS-986156, MK-4166, IN4CAGN01876, GWN323), OX40 (91B12, MOXR 0916, PF-04518600, MED10562, IN4CAGN01949, GSK3174998). In some embodiments, the antibody fragment is, for example, Fab, Fab′, F(ab′)a, Fv, or single-chain Fv.

Table 7 exemplifies immune agonist molecules that are regarded as the targets of the second effector molecules in the conjugate, names of the second effector molecules (e.g., antibodies), and variable region amino acid sequences of the second effector molecules (e.g., antibodies).

TABLE 7
Targets of the immune agonist molecules, names of antibodies,
and variable region amino acid sequences of the antibodies

| | | INN | light chain or | Variable region amino acid |
|---|---|---|---|---|
| Target | Antibody | Num. | heavy chain | sequence of antibodies |
| 4-1BB | Urelumab | 9365 | H | QVQLQQWGAGLLKPSETLSLTCAVYGGSFSGYYWSWI |
| | | | | RQSPEKGLEWIGEINHGGYVTYNPSLESRVTISVDTS |
| | | | | KNQFSLKLSSVTAADTAVYYCARDYGPGNYDWYFDLW |
| | | | | GRG (SEQ ID NO: 75) |
| | | | L | EIVLTQSPATLSLSPGERATLSCRASQSVSSYLAWYQ |
| | | | | QKPGQAPRLLIYDASNRATGIPARFSGSGSGTDFTLT |
| | | | | ISSLEPEDFAVYYCQQRSNWPPALTFCGG |
| | | | | (SEQ ID NO: 76) |
| OX40 | Ivuxolimab | 11055 | H | EVQLVESGGGLVQPGGSLRLSCAASGFTFSSYSMNWV |
| | | | | RQAPGKGLEWVSYISSSSSTIDYADSVKGRFTISRDN |
| | | | | AKNSLYLQMNSLRDEDTAVYYCARESGWYLFDYWGQG |
| | | | | (SEQ ID NO: 77) |
| | | | L | DIQMTQSPSSLSASVGDRVTITCRASQGISSWLAWYQ |
| | | | | QKPEKAPKSLIYAASSLQSGVPSRFSGSGSGTDFTLT |
| | | | | ISSLQPEDFATYYCQQYNSYPPTFGGG |
| | | | | (SEQ ID NO: 78) |
| ICOS | Vopratelimab | 10730 | H | EVQLVESGGGLVQPGGSLRLSCAASGFTFSDYWMDWV |
| | | | | RQAPGKGLVWVSNIDEDGSITEYSPFVKGRFTISRDN |
| | | | | AKNTLYLQMNSLRAEDTAVYYCTRWGRFGFDSWGQG |
| | | | | (SEQ ID NO: 79) |
| | | | L | DIVMTQSPDSLAVSLGERATINCKSSQSLLSGSFNYL |
| | | | | TWYQQKPGQPPKLLIFYASTRHTGVPDRFSGSGSGTD |
| | | | | FTLTISSLQAEDVAVYYCHHHYNAPPTFGPG |
| | | | | (SEQ ID NO: 80) |
| CD27 | Varlilumab | 9933 | H | QVQLVESGGGVVQPGRSLRLSCAASGFTFSSYDMHWV |
| | | | | RQAPGKGLEWVAVIWYDGSNKYYADSVKGRFTISRDN |
| | | | | SKNTLYLQMNSLRAEDTAVYYCARGSGNWGFFDYWGQ |
| | | | | G (SEQ ID NO: 81) |
| | | | L | DIQMTQSPSSLSASVGDRVTITCRASQGISRWLAWYQ |
| | | | | QKPEKAPKSLIYAASSLQSGVPSRFSGSGSGTDFTLT |
| | | | | ISSLQPEDFATYYCQQYNTYPRTFGQG |
| | | | | (SEQ ID NO: 82) |
| TLR2 | Tomaralimab | 11044 | H | EVQLVQSGSELKKPGASVKLSCKASGFTFTTYGINWV |
| | | | | RQAPGQGLEWIGWIYPRDGSTNFNENFKDRATITVDT |
| | | | | SASTAYMELSSLRSEDTAVYFCARLTGGTFLDYWGQG |
| | | | | (SEQ ID NO: 83) |
| | | | L | DIVLTQSPATLSLSPGERATLSCRASESVEYYGTSLM |
| | | | | QWYQQKPGQPPKLLIFGASNVESGVPDRFSGSGSGTD |
| | | | | FTLKISRVEAEDVGMYFCQQSRKLPWTFGGG |
| | | | | (SEQ ID NO: 84) |

In some embodiments, the second effector molecule is an extracellular receptor or a portion of the receptor. The extracellular receptor includes, but is not limited to: a vascular endothelial growth factor receptor (VEGFR), a transforming growth factor βII receptor, CD95, a lymphotoxin β receptor, an interleukin-1 receptor accessory protein, 4-1BBL, Lag-3, activin A receptor type II-like 1 (ALK), AITRL, an IL-15 receptor a (IL15RA), a frizzled class receptor 8 (FZD8), an activin receptor type IIB, an activin A receptor type IIA, GITR, OX40, CD24 or CD40.

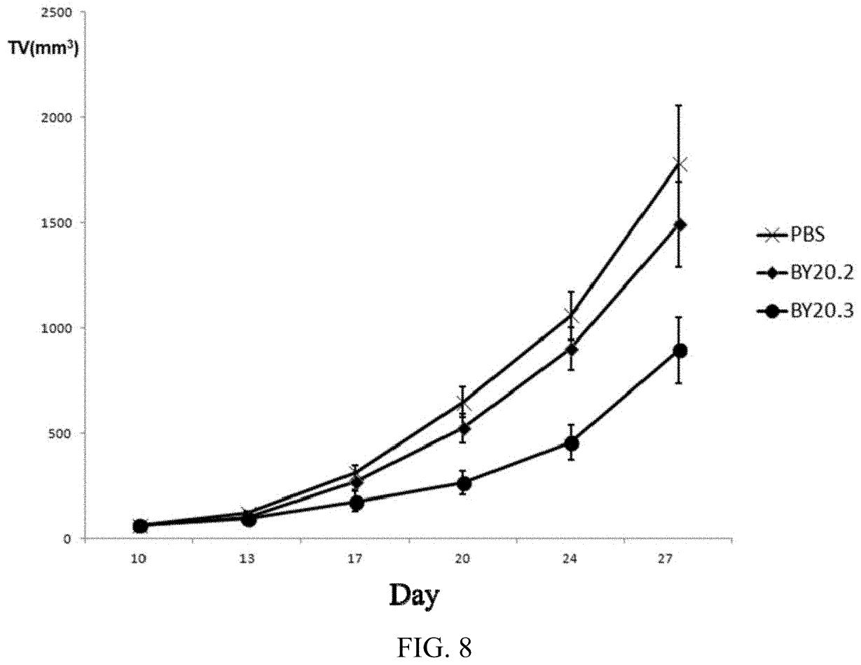

FIG. 8 exemplifies the extracellular receptor or the portion of the receptor that is regarded as the second effector molecule in the conjugate, and an amino acid sequence of an extracellular region thereof.

TABLE 8
Names of extracellular receptors and amino acid sequences of extracellular regions

| Receptors | Amino acid sequences of extracellular regions |
|---|---|
| Transforming | IPPHVQKSVNNDMIVTDNNGAVKFPQLCKFCDVRFSTCDNQKSCMSNCSITSICEKPQEVCV |
| growth factor βII | AVWRKNDENITLETVCHDPKLPYHDFILEDAASPKCIMKEKKKPGETFFMCSCSSDECNDNI |
| receptor | IFSEEYNTSNPD (SEQ ID NO: 85) |
| VEGFR | SDTGRPFVEMYSEIPEIIHMTEGRELVIPCRVTSPNITVTLKKFPLDTLIPDGKRIIWDSRK |
| | GFIISNATYKEIGLLTCEATVNGHLYKTNYLTHRQTNTIIDVVLSPSHGIELSVGEKEVLNC |
| | TARTELNVGIDFNWEYPSSKHQHKKEVNRDLKTQSGSEMKKFLSTLTIDGVTRSDQGEYTCA |
| | ASSGLMTKKNSTFVRVHEK(SEQ ID NO: 86) |
| CD95 | QVTDINSKGLELRKTVTTVETQNLEGLHHDGQFCHKPCPPGERKARDCTVNGDEPDCVPCQE |
| | GKEYTDKAHFSSKCRRCRLCDEGHGLEVEINCTRTQNTKCRCKPNFFCNSTVCEHCDPCTKC |
| | EHGIIKECTLTSNTKCKEEGSRSC (SEQ ID NO: 87) |
| Interleukin 1 | GAARSCRFRGRHYKREFRLEGEPVALRCPQVPYWLWASVSPRINLTWHKNDSARTVPGEEET |
| receptor accessory | RMWAQDGALWLLPALQEDSGTYVCTTRNASYCDKMSIEFRVFENTDAFLPFISYPQILTLST |
| protein | SGVLVCPDLSEFTRDKTDVKIQWYRDSLLLDKDNEKFLSVRGTTHLLVHDVAQEDAGYYRCV |
| | LTFAHEGQQYNITRSIELRIKKKKEETIPVIISPLKTISASLGSRLTIPCKVFLGTGTPLTT |
| | MLWWTANDTHIESAYPGGRVTEGPRQEYSENNENYIEVPLIFDPVTREDLHMDFKCVVHNTL |
| | SFQTLRTTVKEASSTFSWG (SEQ ID NO: 88) |
| Activin receptor-like | DPVKPSRGPLVTCTCESPHCKGPTCRGAWCTVVLVREEGRHPQEHRGCGNLHRELCRGRPTE |
| kinase 1 (ALK1) | FVNHYCCDSHLCNHNVSLVLEATQPPSEQPGTDGQLATGGGTHT (SEQ ID NO: 89) |
| LAG3 | LQPGAEVPVVWAQEGAPAQLPCSPTIPLQDLSLLRRAGVTWQHQPDSGPPAAAPGHPLAPGP |
| | HPAAPSSWGPRPRRYTVLSVGPGGLRSGRLPLQPRVQLDERGRQRGDFSLWLRPARRADAGE |
| | YRAAVHLRDRALSCRLRLRLGQASMTASPPGSLRASDWVILNCSFSRPDRPASVHWFRNRGQ |
| | GRVPVRESPHHHLAESFLFLPQVSPMDSGPWGCILTYRDGFNVSIMYNLTVLGLEPPTPLTV |
| | YAGAGSRVGLPCRLPAGVGTRSFLTAKWTPPGGGPDLLVTGDNGDFTLRLEDVSQAQAGTYT |
| | CHIHLQEQQLNATVTLAIITVTPKSFGSPGSLGKLLCEVTPVSGQERFVWSSLDTPSQRSFS |
| | GPWLEAQEAQLLSQPWQCQLYQGERLLGAAVYFTELSSPGDDDDKGSGSGEPKSC |
| | (SEQ ID NO: 90) |
| AITRL | VSRLEEEMRKLQATVQELQKRLDRLEETVQAKGGGGQLETAKEPCMAKFGPLPSKWQMASSE |
| | PPCVNKVSDWKLEILQNGLYLIYGQVAPNANYNDVAPFEVRLYKNKDMIQTLTNKSKIQNVG |
| | GTYELHVGDTIDLIFNSEHQVLKDNTYWGIILLANPQFIS (SEQ ID NO: 91) |
| IL-15RA | ITCPPPMSVEHADIWVKSYSLYSRERYICNSGFKRKAGTSSLTECVLNKATNVAHWTTPSLK |
| | CIREPKSC (SEQ ID NO: 92) |
| FZD8 | ASAKELACQEITVPLCKGIGYNYTYMPNQFNHDTQDEAGLEVHQFWPLVEIQCSPDLKFFLC |
| | SMYTPICLEDYKKPLPPCRSVCERAKAGCAPLMRQYGFAWPDRMRCDRLPEQGNPDTLCMDY |
| | NRTDLTTEPK (SEQ ID NO: 93) |
| IL1RAP | SERCDDWGLDTMRQIQVFEDEPARIKCPLFEHFLKFNYSTAHSAGLTLIWYWTRQDRDLEEP |
| | INFRLPENRISKEKDVLWFRPTLLNDTGNYTCMLRNTTYCSKVAFPLEVVQKDSCFNSPMKL |
| | PVHKLYIEYGIQRITCPNVDGYFPSSVKPTITWYMGCYKIQNFNNVIPEGMNLSFLIALISN |
| | NGNYTCVVTYPENGRTFHLTRTLTVKVVGSPKNAVPPVIHSPNDHVVYEKEPGEELLIPCTV |
| | YFSFLMDSRNEVWWTIDGKKPDDITIDVTINESISHSRTEDETRTQILSIKKVTSEDLKRSY |
| | VCHARSAKGEVAKAAKVKQKVPAPRYTVEKCKEREEKIILVSSANEIDVRPCPLNPNEHKGT |
| | ITWYKDDSKTPVSTEQASRIHQHKEKLWFVPAKVEDSGHYYCVVRNSSYCLRIKISAKFVEN |
| | EPNLCYNAQAIFKQKLPVAGDGGLVCPYMEFFKNENNELPKLQWYKDCKPLLLDNIHFSGVK |
| | DRLIVMNVAEKHRGNYTCHASYTYLGKQYPITRVIEFITLEENKPTRPVIVSPANETMEVDL |
| | GSQIQLICNVTGQLSDIAYWKWNGSVIDEDDPVLGEDYYSVENPANKRRSTLITVLNISEIE |
| | SRFYKHPFTCFAKNTHGIDAAYIQLIYPVTNSG (SEQ ID NO: 94) |
| GITRL | LQLETAKEPCMAKFGPLPSKWQMASSEPPCVNKVSDWKLEILQNGEYLIYGQVAPNANYNDV |
| | APFEVRLYKNKDMIQTLTNKSKIQNVGGTYELHVGDTIDLIFNSEHQVLKNNTYWGIILLAN |
| | PQFIS (SEQ ID NO: 95) |
| CD24 | SETTTGTSSNSSQSTSNSGLAPNPTNATTK(SEQ ID NO: 96) |

In some embodiments, the second effector molecule is a cytokine, such as IL-1, IL-2, IL-3, IL-4, IL-5, IL-6, IL-7, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28A, IL-281B, IL-29, IL-31, IL-32 and IL-33; a hematopoietic factor such as a macrophage colony-stimulating factor (M-CSF), a granulocyte macrophage colony-stimulating factor (GM-CSF), a granulocyte colony-stimulating factor (G-CSF) and erythropoietin; a tumor necrosis factor (TNF) such as TNF-α and TGF-β; a lymphokine such as lymphotoxin; a modulator for a metabolic process (e.g., leptin); interferon (e.g., IFN-α, IFN-β and IFN-γ); and a chemokine. In some embodiments of the present invention, the cytokine is an interleukin, such as IL-2, IL-7, IL-15 or IL-33.

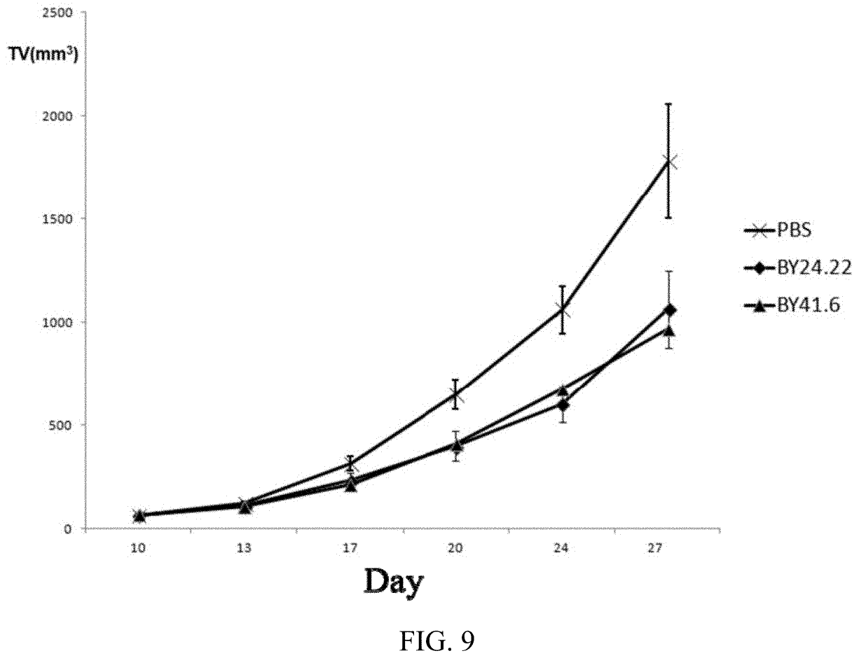

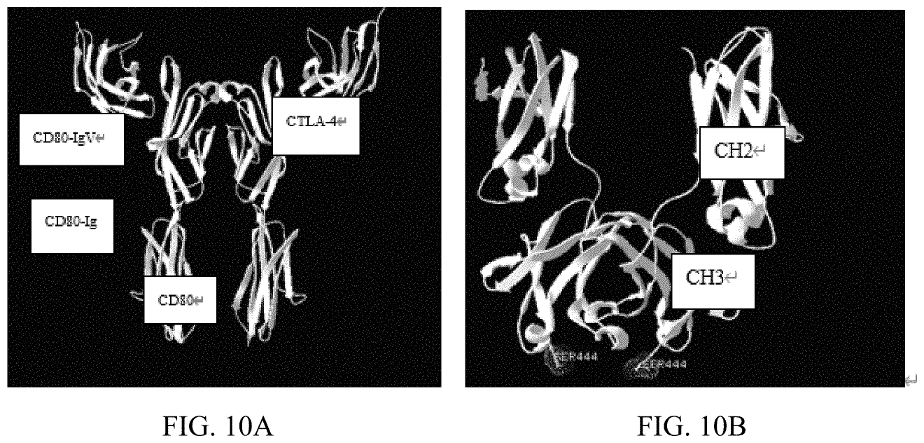

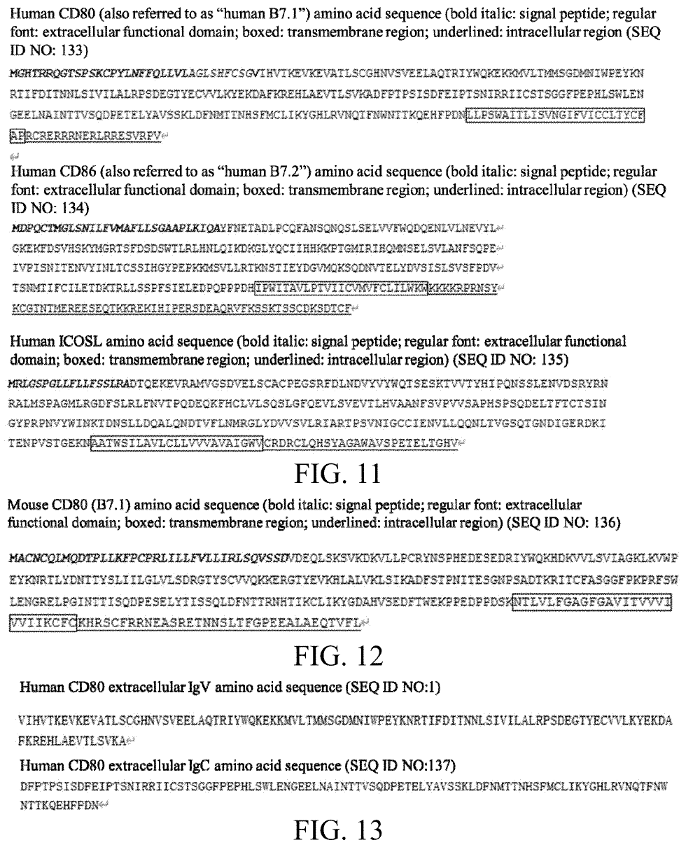

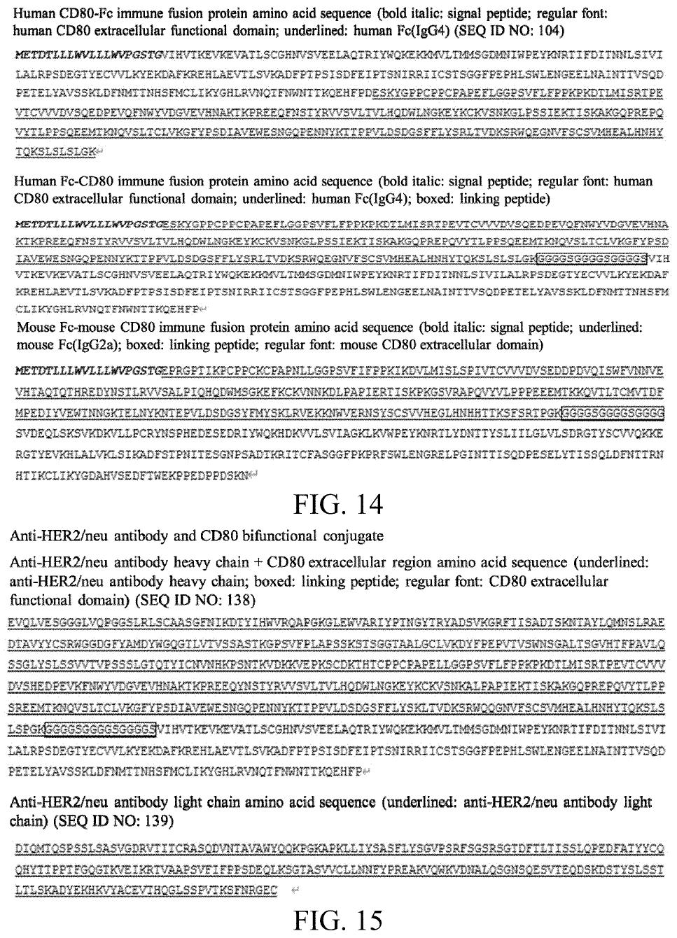

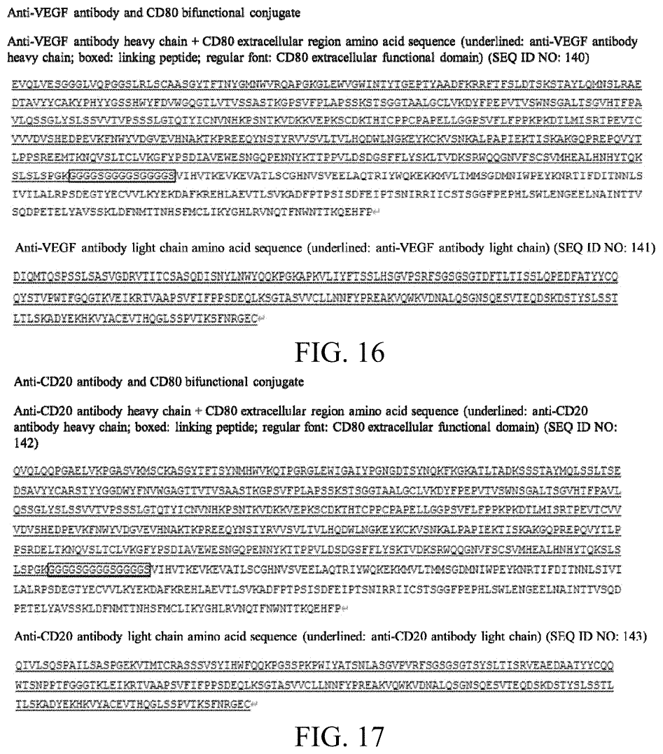

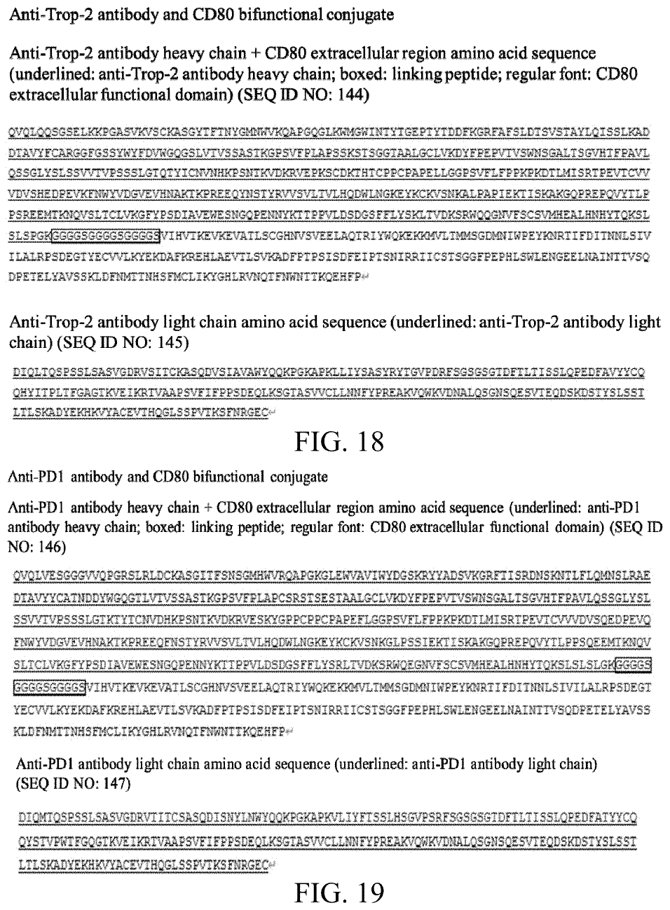

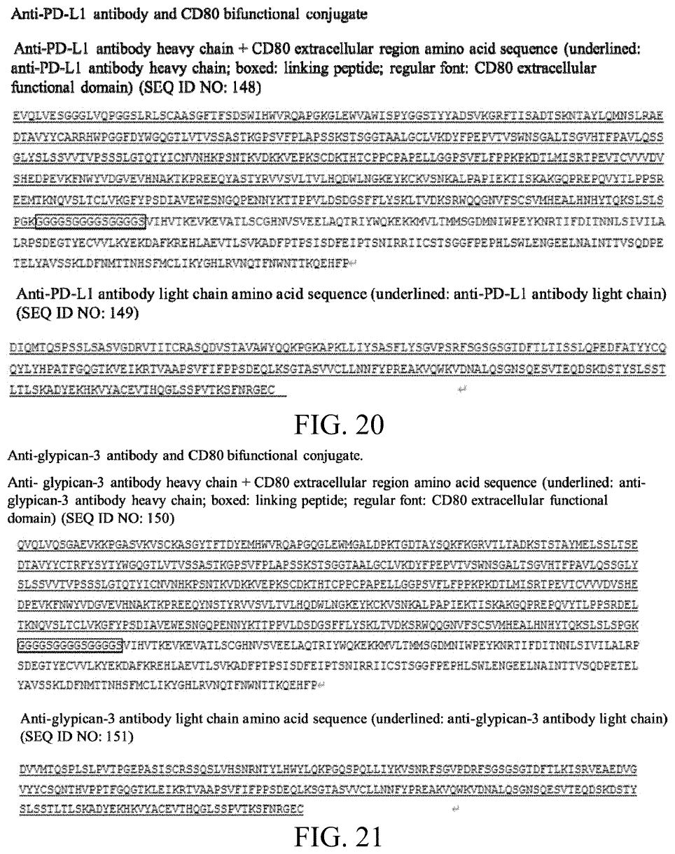

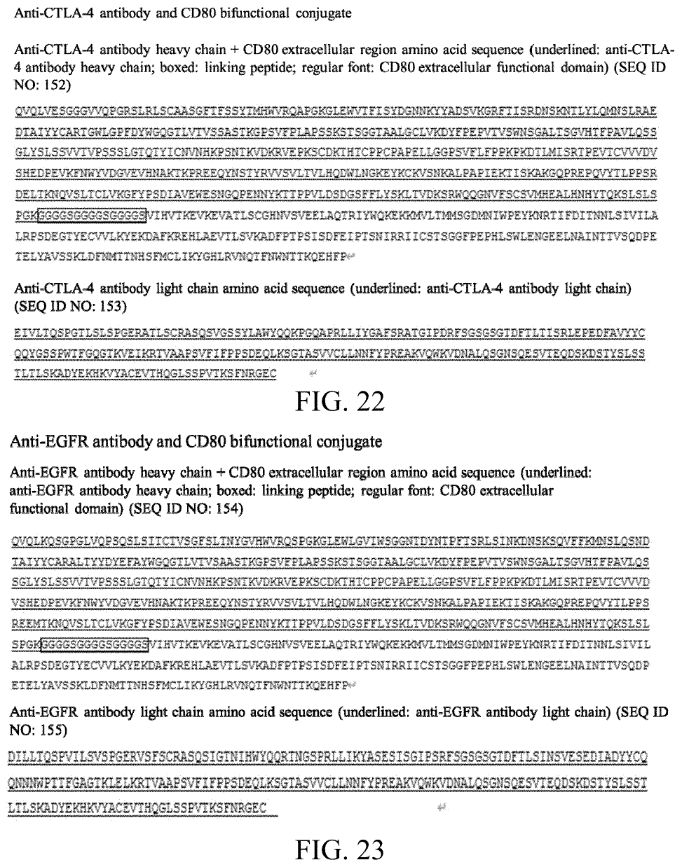

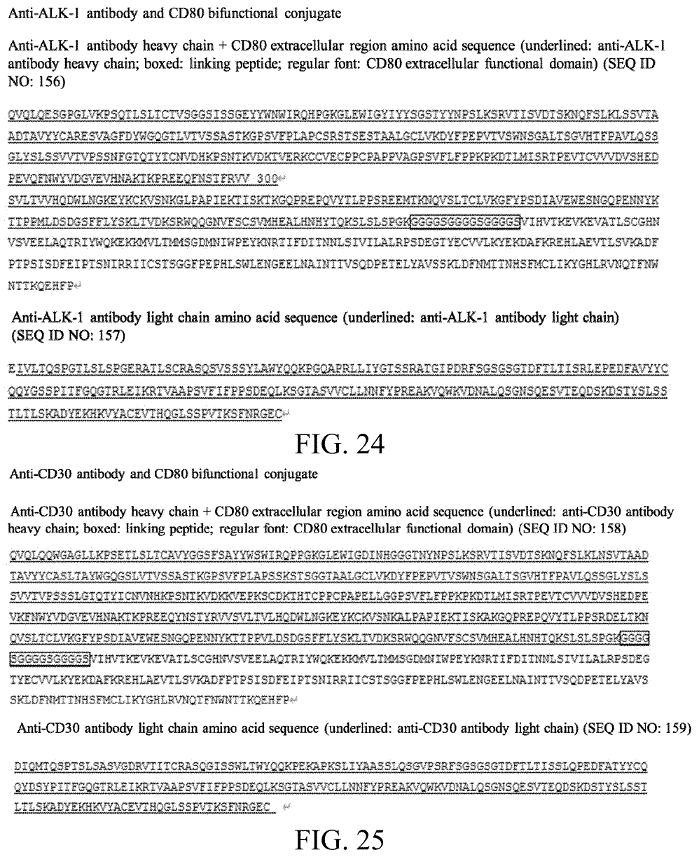

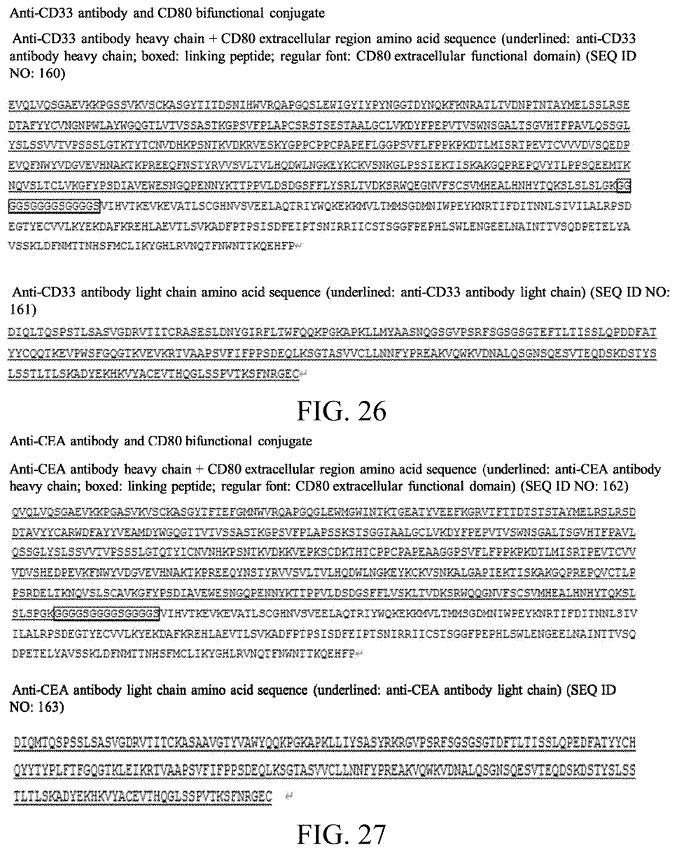

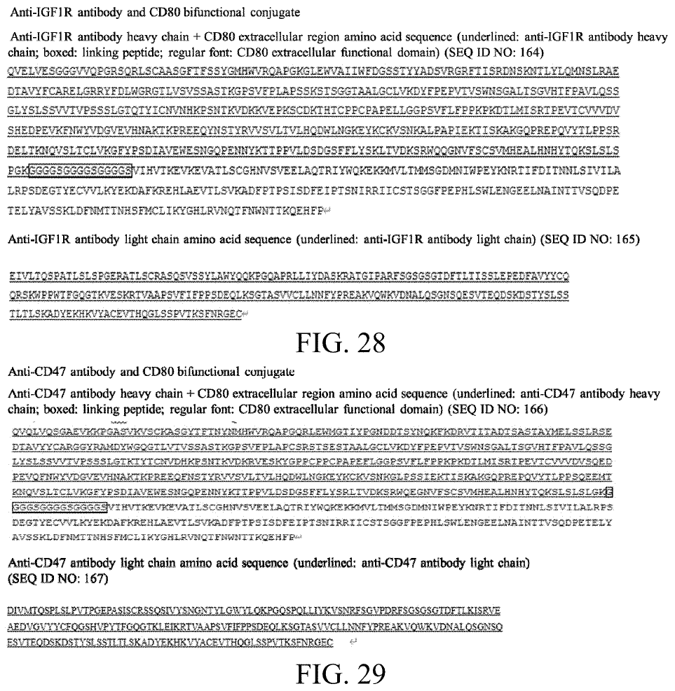

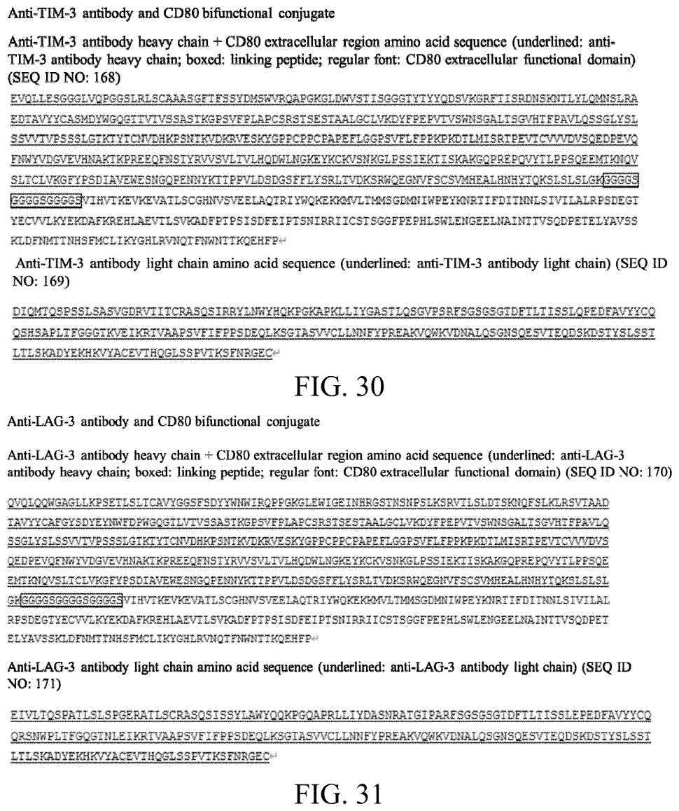

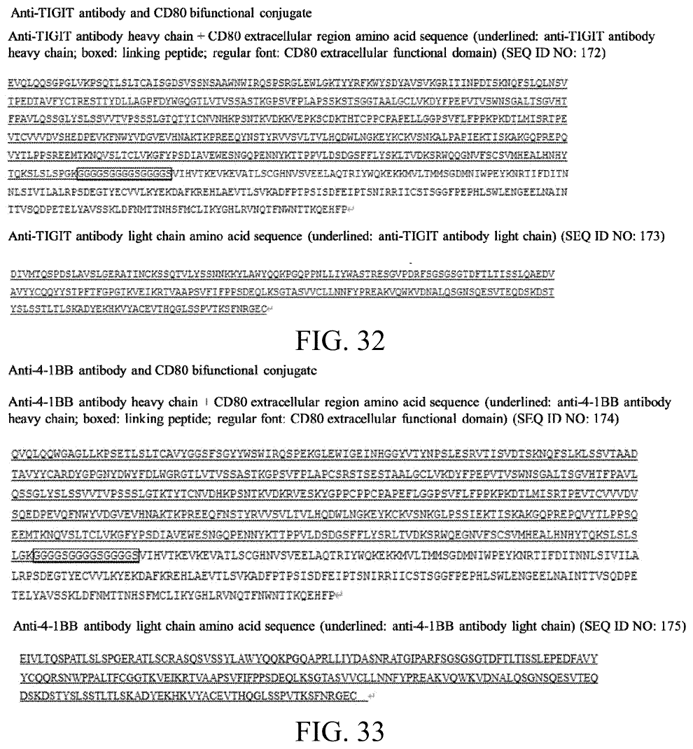

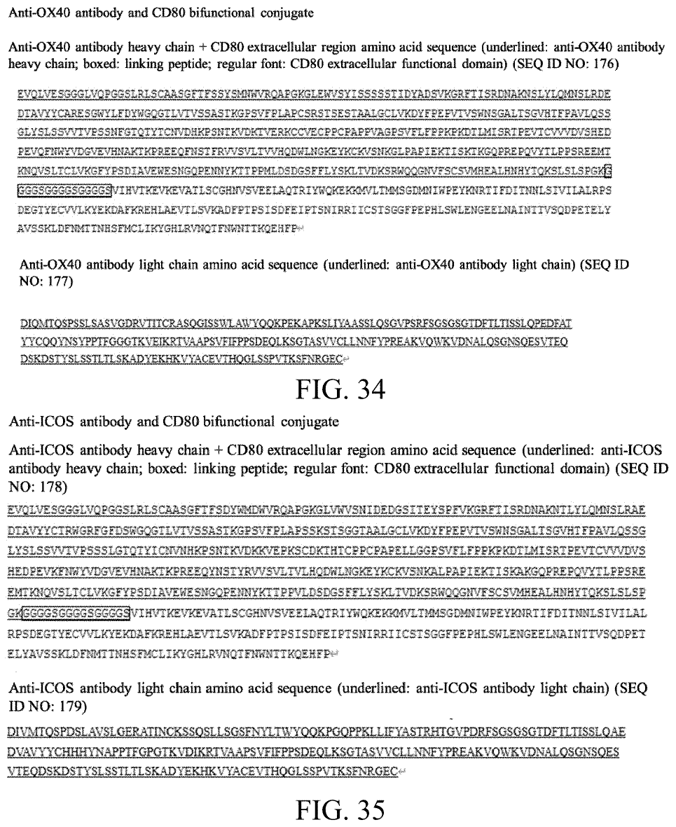

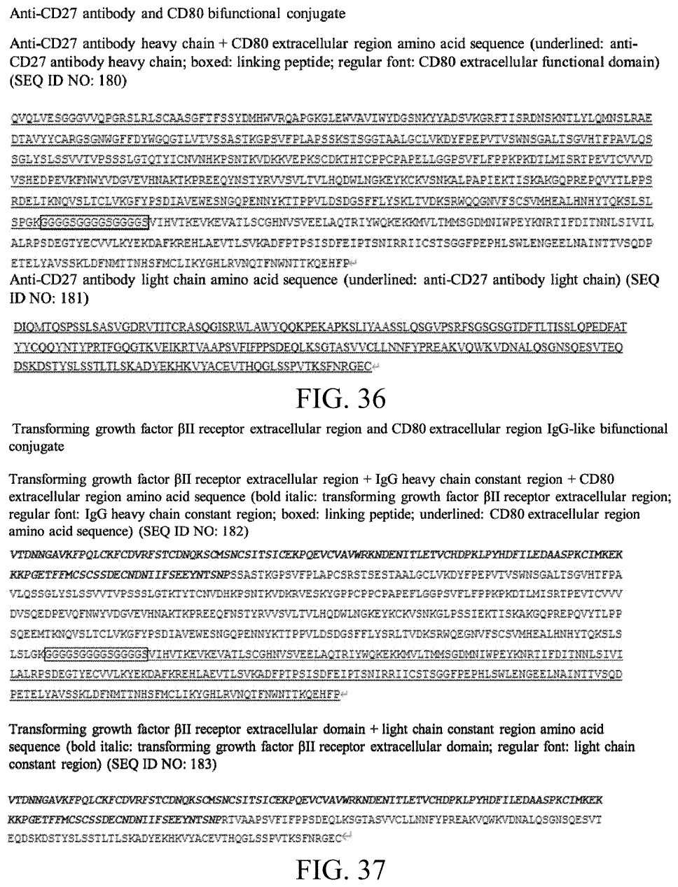

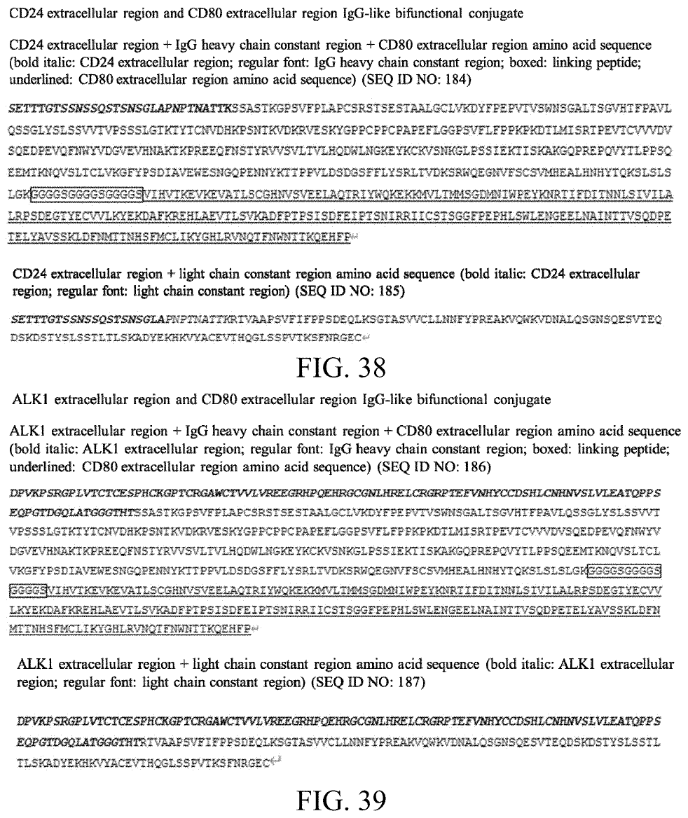

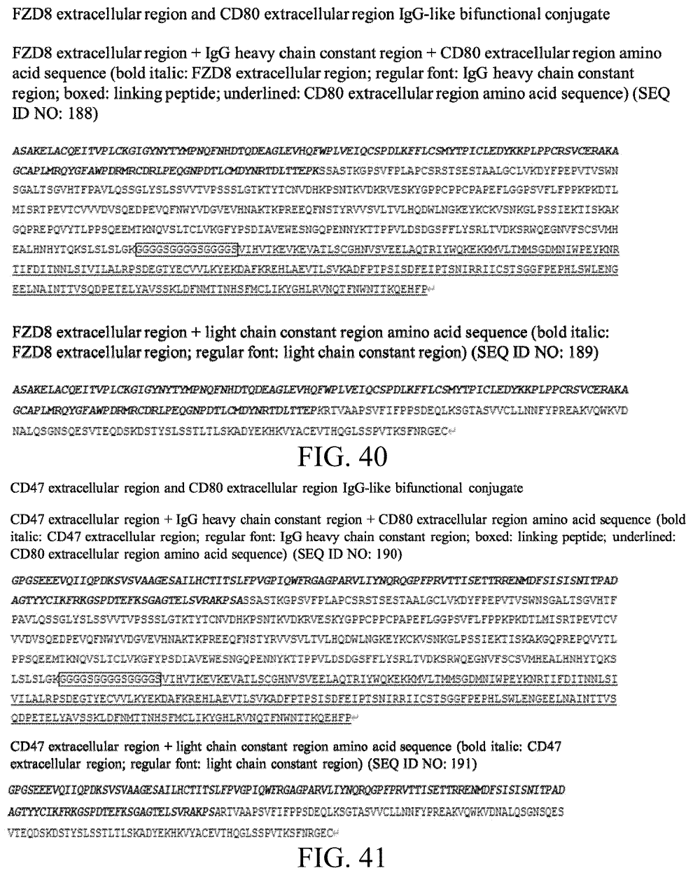

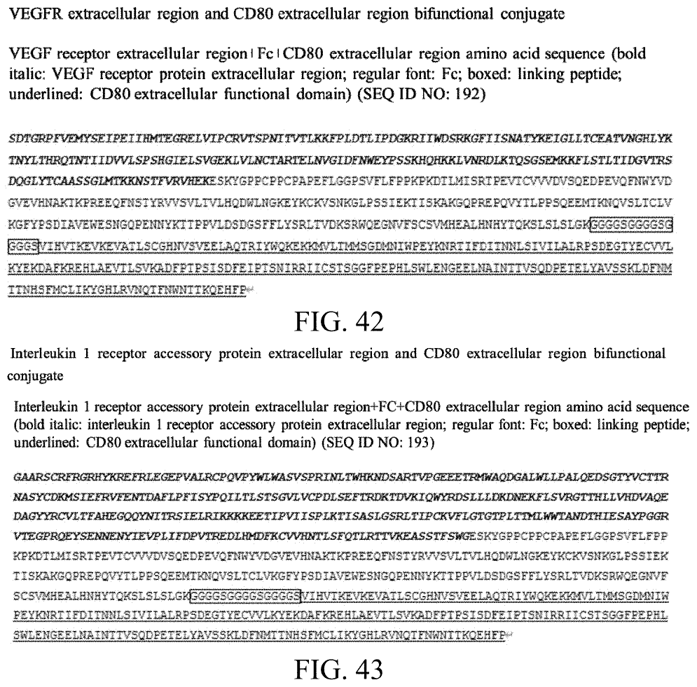

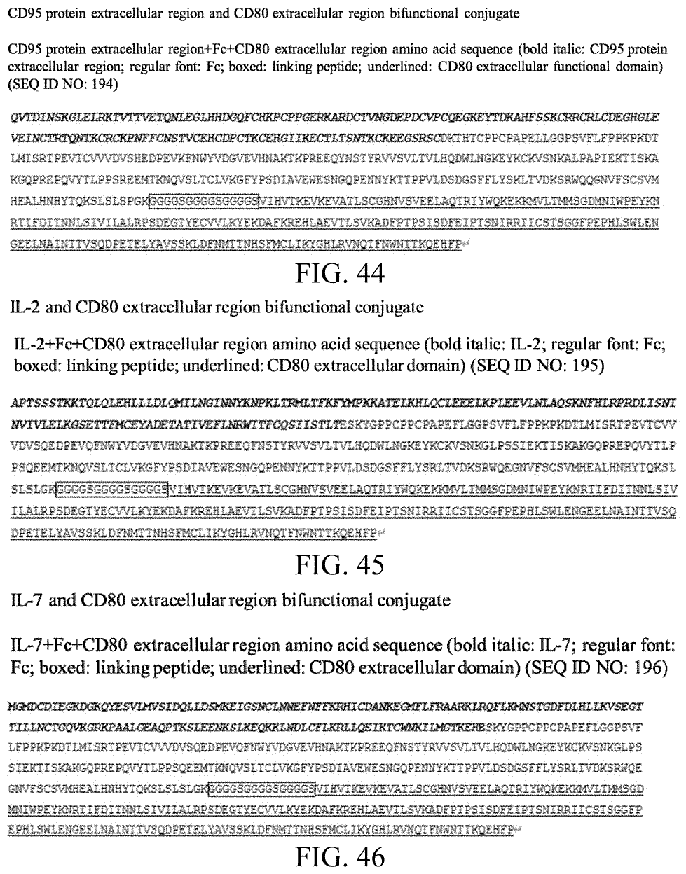

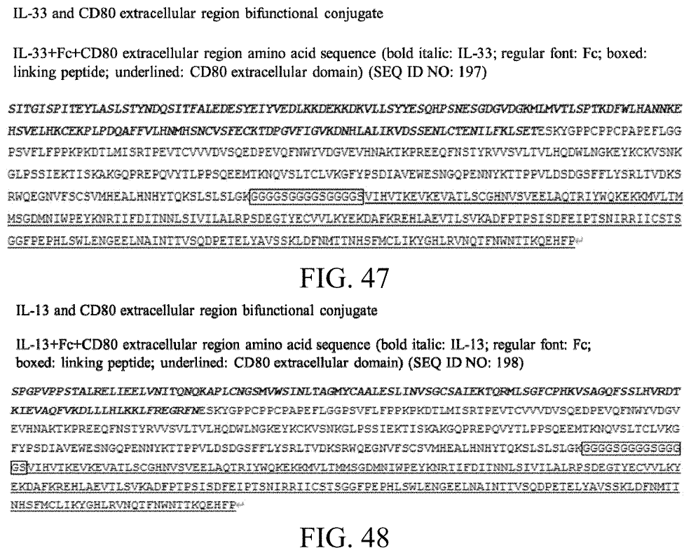

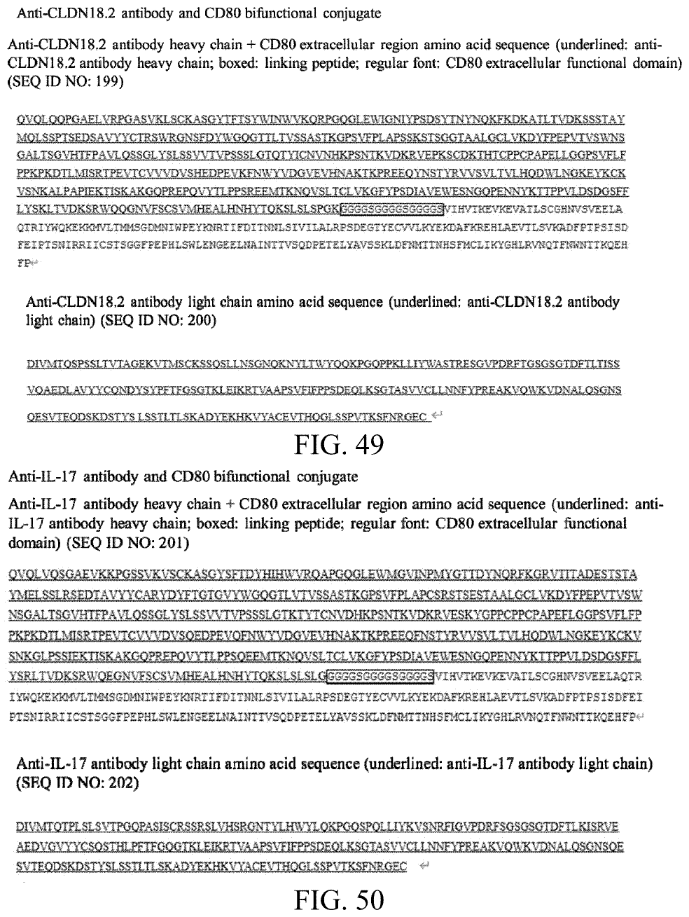

FIG. 9 exemplifies names of cytokins that are regarded as the second effector molecules in the conjugate, and amino acid sequences thereof.

FIG. 9 Names of exemplified cytokins and amino acid sequences thereof.

| Cytokins | Amino acid sequences |
|---|---|
| IL-2 | APTSSSTKKTQLQLEHLLLDLQMILNGINNYKNPKLTRMLTFKFYMPKKATELKHL |
| | QCLEEELKPLEEVLNLAQSKNFHLRPRDLISNINVIVLELKGSETTFMCEYADETATI |
| | VEFLNRWITFCQSIISTLT (SEQ ID NO: 97) |
| IL-7 | MGMDCDIEGKDGKQYESVLMVSIDQLLDSMKEIGSNCLNNEFNFFKRHICDANKE |
| | GMFLFRAARKLRQFLKMNSTGDFDLHLLKVSEGTTILLNCTGQVKGRKPAALGEA |
| | QPTKSLEENKSLKEQKKLNDLCFLKRLLQEIKTCWNKILMGTKEH (SEQ ID NO: 98) |
| IL-10 | SPGQGTQSENSCTHFPGNLPNMLRDLRDAFSRVKTFFQMKDQLDNLLLKESLLED |
| | FKGYLGCQALSEMIQFYLEEVMPQAENQDPDIKAHVNSLGENLKTLRLRLRRCHR |
| | FLPCENKSKAVEQVKNAFNKLQEKGIYKAMSEFDIFINYIEAYMTMKIRN (SEQ ID |
| | NO: 99) |
| IL-33 | SITGISPITEYLASLSTYNDQSITFALEDESYEIYVEDLKKDEKKDKVLLSYYESQHP |
| | SNESGDGVDGKMLMVTLSPTKDFWLHANNKEHSVELHKCEKPLPDQAFFVLHN |
| | MHSNCVSFECKTDPGVFIGVKDNHLALIKVDSSENLCTENILFKLSET (SEQ ID |
| | NO: 100) |

In one embodiment, the N-terminal of the conjugate of the present invention is an antibody bevacizumab that can bind to VEGF. The conjugate retains the binding ability of the bevacizumab antibody to VEGF, and has a better inhibitory effect on tumor growth; the conjugate is administered in combination with a PD1 antibody, and has a better anti-tumor effect than that of single administration; and the anti-tumor effect of this conjugate is closely related to the recruitment of T lymphocytes to infiltrate the interior of a tumor and the inhibition of tumor neovascularization.

In one embodiment, the N-terminal of the conjugate of the present invention is an antibody codrituzumab that can bind to GPC-3. The conjugate retains the binding ability of the codrituzumab antibody to GPC-3, and has a better tumor inhibition effect than that of codrituzumab.

In one embodiment, the N-terminal of the conjugate of the present invention is an antibody trastuzumab that can bind to HER2. The conjugate retains the binding ability of the trastuzumab antibody to HER2.

In one embodiment, the N-terminal of the conjugate of the present invention is an antibody sacituzumab that can bind to trop-2. The conjugate retains the binding ability of the sacituzumab antibody to trop-2.

In one embodiment, The N-terminal of the conjugate of the present invention is VEGFR that can bind to VEGF, or an extracellular domain thereof. The conjugate retains the binding ability of VEGFR to VEGF, and has a better effect of inhibiting tumor growth.

In one embodiment, the N-terminal of the conjugate of the present invention is TGF-βRII which can bind to TGF-β1, or an extracellular domain thereof. The conjugate can bind to TGF-β1 at high affinity and has a better effect of inhibiting tumor growth.

Furthermore, similar to CD80, CD86 and ICOSL are members of the immunoglobulin superfamily, and their extracellular domains are each composed of an IgV domain and an IgC (immunoglobulin constant) domain.

Both CD80 and CD86 can bind to CD28 and CTLA-4, but CD86 cannot bind to PD-L1. ICOS can also bind to CD28 and CTLA-4 (Liu W., et al., Adv Exp Med Biol. 2019; 1172: 63-78).

Therefore, although the present invention only exemplifies the Fc-CD80 fusion protein and the conjugate containing the Fc-CD80 fusion protein in the examples, the present invention also contemplates the technical solutions after CD80 is replaced with CD86 or ICOS, for example, a Fc-CD86 fusion protein and a conjugate containing the Fc-CD86 fusion protein; a Fc-ICOS fusion protein and a conjugate containing the Fc-ICOS fusion protein.

### III. Production and Purification of the Fc-CD80 Fusion Protein and the Conjugate Thereof

The Fc-CD80 fusion protein of the present invention and its conjugate may be, for example, obtained by solid-state peptide synthesis (e.g., Merrifield solid-phase synthesis) or recombinant production. For recombinant production, a polynucleotide that encodes each subunit of the Fc-CD80 fusion protein or conjugate thereof is isolated and inserted into one or more vectors for further cloning and/or expression in host cells. The polynucleotide can be readily isolated and sequenced using conventional methods. In one embodiment, a vector containing one or more polynucleotides of the present invention, preferably, an expression vector is provided.

The expression vector may be constructed using methods that are well-known to those skilled in the art. The expression vector includes, but is not limited to, viruses, plasmids, cosmids, a phage, or yeast artificial chromosomes (YACs). In a preferred embodiment, a glutamine synthase high-performance expression vector having a dual expression box is used.

Once an expression vector containing one or more polynucleotides of the present invention has been prepared for expression, the expression vector can be transfected or introduced into a suitable host cell. A variety of techniques can be used to accomplish this, e.g., protoplast fusion, calcium phosphate precipitation, electroporation, retroviral transduction, viral transfection, gene gun, liposome-based transfection, or other conventional techniques.

In one embodiment, a host cell containing one or more polynucleotides of the present invention is provided. In one embodiment, a host cell containing the expression vector of the present invention is provided. As used herein, the term “host cell” refers to any kind of cell system that can be engineered to produce the Fc-CD80 fusion protein of the present invention or the conjugate thereof. The host cell which is suitable for replicating and supporting the expression of the Fc-CD80 fusion protein or its conjugate of the present invention are well-known in the art. As needed, such cells may be transfected or transduced with specific expression vectors, and a large number of vector-containing cells may be cultured for inoculating a large-scale fermenter to obtain sufficient amounts of the Fc-CD80 fusion protein or its conjugate of the present invention for clinical applications. Suitable host cells include prokaryotic microorganisms such as E. coli, eukaryotic microorganisms such as filamentous fungi or yeast, or various eukaryotic cells such as Chinese hamster ovary cells (CHO) and insect cells. A mammalian cell line suitable for suspension culture can be used. Examples of useful mammalian host cell lines include a monkey kidney CV1 line (COS-7) transformed by SV40; a human embryonic kidney line (HEK 293 or 293F cells), baby hamster kidney cells (BHK), monkey kidney cells (CV1), African green monkey kidney cells (VERO-76), human cervical cancer cells (HELA), canine kidney cells (MDCK), Buffalo rat liver cells (BRL 3A), human lung cells (W138), human liver cells (Hep G2), CHO cells, NSO cells, and myeloma cell lines such as YO, NSO, P3X63 and Sp2/0. For a review of mammalian host cell lines suitable for protein production, see e.g., Yazaki and Wu, Methods in Molecular Biology, vol. 248 (edited by B.K.C. Lo, Humana Press, Totowa, N.J.), pp. 255-268 (2003). In a preferred embodiment, the host cell is a CHO, HEK293 or NSO cell.

Standard techniques for expressing foreign genes in these host cell systems are known in the art. In one embodiment, provided is a method for producing the Fc-CD80 fusion protein or this conjugate of the present invention. The method includes: culturing the host cell as provided herein under conditions suitable for the expression of the Fc-CD80 fusion protein or this conjugate, wherein the host cell includes a polynucleotide that encodes the Fc-CD80 fusion protein or the conjugate thereof, and the Fc-CD80 fusion protein or the conjugate thereof is recovered from the host cell (or a host cell culture medium).

The Fc-CD80 fusion protein or the conjugate thereof prepared as described herein can be purified by known prior arts such as high-performance liquid chromatography, ion exchange chromatography, gel electrophoresis, affinity chromatography, size exclusion chromatography and other purification means. The actual conditions used to purify a particular protein will also depend on factors such as net charge, hydrophobicity, and hydrophilicity, and these will be apparent to those skilled in the art.

The purity of the Fc-CD80 fusion protein or its conjugate of the present invention can be determined by any of a variety of well-known analytical methods, including gel electrophoresis, high-performance liquid chromatography, and the like. The Fc-CD80 fusion protein or the conjugate thereof provided herein, can be identified, screened, or characterized for their physical/chemical properties and/or biological activities by a variety of assays known in the art.

### IV. Combination Therapy

PD-1 is an immunosuppressive protein that has two ligands, i.e., PD-L1 and PD-L2, respectively. The interaction between PD-1 and PD-L1 is known to cause, for example, a decrease in tumor-infiltrating lymphocytes and/or immune evasion of cancer cells. Immunosuppression can be reversed by inhibiting the local interaction between PD-1 and PD-L1 or PD-L2; and this effect is additive when the interaction between PD-1 and PD-L2 is also blocked (Iwai Y., et al., Involvement of PD-L1 on tumor cells in the escape from host immune system and tumor immunotherapy by PD-L1 blockade, Proc. Nat'l. Acad. Sci. USA, 2002, 99: 12293-7). In view of the importance of the signal transduction of an immune checkpoint PD-1 in modulating immune responses, the present invention has developed a pharmaceutical composition for combined therapy, the pharmaceutical composition including an Fc-CD80 fusion protein and its conjugate of the present invention, and an anti-PD-1 antibody.

Compared to monotherapy using the Fc-CD80 fusion protein or its conjugate of the present invention or monotherapy using an anti-PD-1 antibody, the pharmaceutical composition described herein for combined therapy can provide superior beneficial effects, such as enhanced anticancer effects, reduced toxicity and/or reduced side effects. For example, the Fc-CD80 fusion protein or its conjugate of the present invention and/or the anti-PD-1 antibody in the pharmaceutical composition can be administered at lower doses or for shorter administration time than those required to achieve the same therapeutic effect as the administration using the monotherapy. Therefore, the present invention also discloses use of the pharmaceutical composition for combined therapy for the treatment of cancers. The effectiveness of the foregoing pharmaceutical composition can be detected in cell models and animal models known in the art.

The anti-PD-1 antibody contained in the combined therapy may be any anti-PD-1 antibody, as long as it is an antibody capable of inhibiting or reducing the binding of PD-1 to its ligand, including anti-PD-1 antibodies known in the prior art and anti-PD-1 antibodies developed in the future. The anti-PD-1 antibody is capable of specifically binding to PD-1 at high affinity, for example at a K_{D} of 10⁻⁸ M or less, preferably 10⁻⁹ M to 10⁻² M, thereby blocking a signal transduction pathway mediated by the binding of PD-1 to ligands PD-L1 and/or PD-L2.

### V. Pharmaceutical Compositions

The pharmaceutical composition of the present invention may include a “therapeutically effective amount” or a “prophylactically effective amount” of the Fc-CD80 fusion protein or its conjugate of the present invention. “Therapeutically effective amount” refers to an amount for effective achievement of the desired therapeutic result at a required dose and for a required period of time. The effective amount of treatment can vary based on a variety of factors such as disease status, individual age, sex, and weight. The therapeutically effective amount refers to any amount at which toxic or harmful effects are less than beneficial effects of treatment. “Therapeutically effective amount” preferably inhibits a measurable parameter (e.g., a tumor growth rate) by at least about 20%, more preferably at least about 40%, even more preferably at least about 60%, and still more preferably at least about 80%, relative to an untreated subject. The ability of the pharmaceutical composition of the present invention to inhibit measurable parameters (e.g., tumor volume) may be evaluated in an animal model system that predicts efficacies in human tumors.

“Prophylactically effective amount” refers to an amount for effective achievement of the desired therapeutic result at a required dose and for a required period of time. Typically, a prophylactically effective amount is less than a therapeutically effective amount because a prophylactic dose is administered in a subject prior to or at an earlier stage of a disease.

### VI. Uses of the Fc-CD80 Fusion Protein, a Conjugate Thereof and a Pharmaceutical Composition Including the Fc-CD80 Fusion Protein or the Conjugate Thereof

The Fc-CD80 fusion protein, the conjugate thereof and the pharmaceutical composition disclosed herein have therapeutic and prophylactic uses for cancers. For example, the Fc-CD80 fusion protein, the conjugate thereof and the pharmaceutical composition may be administered to cultured cells in vitro or ex vivo or administered to a subject, e.g., a human subject, to treat and/or prevent a variety of cancerous diseases.

In one aspect, the present invention relates to a method for inhibiting tumor cell growth in a subject in vivo using an Fc-CD80 fusion protein, a conjugate thereof, or a pharmaceutical composition, the method including: administering to the subject a therapeutically effective amount of the Fc-CD80 fusion protein, the conjugate thereof, or the pharmaceutical composition described above. In another embodiment, the present invention provides a method for preventing the appearance or metastasis or recurrence of tumor cells in a subject, the method including: administering to the subject a prophylactically effective amount of the Fc-CD80 fusion protein, the conjugate thereof, or the pharmaceutical composition described above.

In some embodiments, cancers treated and/or prevented with the Fc-CD80 fusion protein, the conjugate thereof, or the pharmaceutical composition include, but are not limited to, solid tumors, hematological cancers (e.g., leukemia, lymphoma, myeloma, such as, multiple myeloma) and metastatic lesions. In one embodiment, the cancers are solid tumors. Examples of solid tumors include malignant tumors, e.g., sarcomas and carcinomas of multiple organ systems, such as those that invade the lungs, breast, ovaries, lymphoids, gastrointestinal tract (e.g., colon), anus, genitals, and genitourinary tract (e.g., kidney, bladder epithelium, bladder cells, prostate), pharynx, CNS (e.g., brain, neurological cells, or glial cells), head and neck, skin (e.g., melanoma), nasopharynx (e.g., differentiated or undifferentiated metastatic or locally recurrent nasopharyngeal carcinoma) and pancreas, and adenocarcinoma, including malignant tumors such as colon cancer, rectum cancer, renal cell cancer, liver cancer, non-small cell lung cancer, small bowel cancer and esophageal cancer. Cancers may be early, intermediate or advanced or metastatic cancers.

In some embodiments, the cancer is selected from melanoma, breast cancer, colon cancer, esophageal cancer, gastrointestinal stromal tumor (GIST), kidney cancer (e.g., renal cell cancer), liver cancer, non-small cell lung cancer (NSCLC), ovarian cancer, pancreatic cancer, prostate cancer, head and neck tumors, gastric cancer, hematologic malignancies (e.g., lymphoma).

The following examples are described to assist in the understanding of the present invention. The examples are not intended and should not be construed in any way to limit the protection scope of the present invention.

### EXAMPLES

### Example 1. Construction, Expression, Purification and Specific Target Binding Ability Test of a Fc-hCD80 Fusion Protein

### Example 1.1. Construction of Expression Vector for Fc-hCD80 Fusion Protein

In this example, an expression vector for the Fc-hCD80 fusion protein is constructed. As its control, an expression vector for a hCD80-Fc fusion protein is also constructed.

1.1.1 Synthesis of an Encoding Nucleotide of a Fusion Protein in which the N-Terminal of the Human CD80 Extracellular Domain is Linked to the C-Terminal of Fc

According to the sequence of the CD80 extracellular domain in Table 1 and the human IgG4 Fc sequence in Table 4, the nucleotide sequence is optimized to be suitable for expression in Chinese hamster ovary cancer cells (CHO), and Shanghai Jierui Bioengineering Co., Ltd. is entrusted to synthesize the following polynucleotide sequence of SEQ ID NO: 101. The Fc-hCD80 fusion protein produced after the expression of the nucleotide sequence is also represented herein as a fusion protein BY24.30.

Nucleotide sequence of the fusion protein BY24.30 (Fc-hCD80)
(SEQ ID NO: 101)
CTCGAGGCCACCATGGAAACCGACACACTGCTGCTGTGGGTCCTGCTGCTGTGGGTGCCAGGCT
CTACAGGCGAGTCCAAGTACGGCCCTCCTTGCCCTCCTTGCCCTGCCCCTGAGTTCCTGGGCGGC
CCTTCCGTGTTCCTGTTCCCTCCTAAGCCTAAGGACACCCTGATGATCTCCCGCACCCCTGAGGTG
ACCTGCGTGGTGGTGGACGTGTCCCAGGAGGACCCTGAGGTGCAGTTCAACTGGTACGTGGACG
GCGTGGAGGTGCACAACGCCAAGACCAAGCCTCGCGAGGAGCAGTTCAACTCCACCTACCGCGT
GGTGTCCGTGCTGACCGTGCTGCACCAGGACTGGCTGAACGGCAAGGAGTACAAGTGCAAGGTG
TCCAACAAGGGCCTGCCTTCCTCCATCGAGAAGACCATCTCCAAGGCCAAGGGCCAGCCTCGCG
AGCCTCAGGTGTACACCCTGCCTCCTTCCCAGGAGGAGATGACCAAGAACCAGGTGTCCCTGAC
CTGCCTGGTGAAGGGCTTCTACCCTTCCGACATCGCCGTGGAGTGGGAGTCCAACGGCCAGCCTG
AGAACAACTACAAGACCACCCCTCCTGTGCTGGACTCCGACGGCTCCTTCTTCCTGTACTCCCGC
CTGACCGTGGACAAGTCCCGCTGGCAGGAGGGCAACGTGTTCTCCTGCTCCGTGATGCACGAGG
CCCTGCACAACCACTACACCCAGAAGTCCCTGTCCCTGTCCCTGGGCAAGGGAGGCGGAGGATC
TGGCGGCGGAGGCAGTGGAGGCGGCGGAAGTGTGATCCATGTAACAAAAGAAGTGAAGGAAGT
GGCTACACTCTCTTGCGGCCACAACGTGTCCGTGGAGGAACTAGCTCAGACCCGGATCTATTGGC
AGAAAGAAAAGAAGATGGTGCTGACCATGATGTCCGGCGACATGAACATTTGGCCAGAGTACAA
GAACCGCACAATTTTCGACATTACAAACAACCTCTCTATTGTGATTCTGGCTCTCAGGCCTAGCGA
CGAGGGCACATACGAGTGCGTGGTGCTCAAGTACGAGAAGGACGCTTTCAAGCGGGAGCACCTC
GCTGAGGTGACCCTGTCCGTGAAGGCCGACTTCCCTACTCCATCTATCTCCGACTTCGAGATCCCT
ACCTCTAACATCAGGCGGATCATCTGCAGCACCTCCGGCGGCTTCCCAGAGCCTCACCTCTCTTG
GCTGGAGAACGGCGAGGAGCTGAACGCTATCAACACCACAGTGTCTCAGGACCCTGAGACCGAG
CTGTACGCCGTGTCCTCTAAGCTCGACTTCAACATGACCACAAACCACTCCTTCATGTGCCTGATC
AAGTACGGCCACCTGAGAGTGAACCAGACATTCAACTGGAACACAACCAAGCAGGAGCACTTCC
CATAAGAATTC
Amino acid sequence of the fusion protein BY24.30 (Fc-hCD80, IgG4)
(SEQ ID NO: 102)
METDTLLLWVLLLWVPGSTGESKYGPPCPPCPAPEFLGGPSVFLFPPKPKDTLMISRTPEVTCVVVDV
SQEDPEVQFNWYVDGVEVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKGGGGSGGGGSGGGGSVIHV
TKEVKEVATLSCGHNVSVEELAQTRIYWQKEKKMVLTMMSGDMNIWPEYKNRTIFDITNNLSIVILA
LRPSDEGTYECVVLKYEKDAFKREHLAEVTLSVKADFPTPSISDFEIPTSNIRRIICSTSGGFPEPHLSW
LENGEELNAINTTVSQDPETELYAVSSKLDFNMTTNHSFMCLIKYGHLRVNQTFNWNTTKQEHFP
The amino acid sequence ″METDTLLLWVLLLWVPGSTG″ is a signal peptide.

1.1.2 Synthesis of an Encoding Nucleotide of a Control Fusion Protein in which the C-Terminal of the Human CD80 Extracellular Domain is Linked to the N-Terminal of Fc

Similar to the above example 1.1.1, the following polynucleotide sequence of SEQ ID NO: 103 is synthesized. The hCD80-Fc fusion protein produced after the expression of the nucleotide sequence is also represented herein as a fusion protein BY24.23 as a control.

Nucleotide sequence of the fusion protein BY24.23 (hCD80-Fc, IgG4)
(SEQ ID NO: 103)
CTCGAGGCCACCATGGAGACCGACACACTCCTCCTGTGGGTGCTGCTGCTGTGGGTGCCTGGCTC
CACTGGCGTCATCCATGTGACCAAAGAGGTCAAAGAAGTCGCAACTCTGTCTTGCGGACACAAC
GTCTCCGTCGAAGAACTCGCCCAGACTAGAATATATTGGCAGAAGGAAAAGAAGATGGTGCTCAC
CATGATGTCCGGCGATATGAACATTTGGCCTGAGTACAAGAACCGGACAATCTTCGATATTACTAAT
AACCTGAGTATCGTGATTCTCGCTCTGCGCCCTAGCGACGAGGGCACATACGAGTGTGTGGTGCT
GAAGTACGAGAAGGATGCATTCAAGCGGGAGCACCTCGCAGAGGTGACACTCTCCGTGAAGGCC
GACTTCCCAACCCCATCTATCTCCGACTTCGAGATCCCTACCTCTAACATCAGGCGGATCATCTGC
AGCACCTCCGGCGGCTTCCCAGAGCCTCACCTCTCTTGGCTGGAGAACGGCGAGGAGCTGAACG
CTATCAACACCACAGTGTCTCAGGACCCTGAGACCGAGCTGTACGCCGTGTCCTCTAAGCTCGAC
TTCAACATGACCACAAACCACTCCTTCATGTGCCTGATCAAGTACGGCCACCTGAGAGTGAACCA
GACATTCAACTGGAACACAACCAAGCAGGAGCACTTCCCAGACGAGTCCAAGTACGGCCCTCCT
TGCCCTCCTTGCCCTGCCCCTGAGTTCCTGGGCGGCCCTTCCGTGTTCCTGTTCCCTCCTAAGCCT
AAGGACACCCTGATGATCTCCCGCACCCCTGAGGTGACCTGCGTGGTGGTGGACGTGTCCCAGG
AGGACCCTGAGGTGCAGTTCAACTGGTACGTGGACGGCGTGGAGGTGCACAACGCCAAGACCA
AGCCTCGCGAGGAGCAGTTCAACTCCACCTACCGCGTGGTGTCCGTGCTGACCGTGCTGCACCA
GGACTGGCTGAACGGCAAGGAGTACAAGTGCAAGGTGTCCAACAAGGGCCTGCCTTCCTCCATC
GAGAAGACCATCTCCAAGGCCAAGGGCCAGCCTCGCGAGCCTCAGGTGTACACCCTGCCTCCTT
CCCAGGAGGAGATGACCAAGAACCAGGTGTCCCTGACCTGCCTGGTGAAGGGCTTCTACCCTTC
CGACATCGCCGTGGAGTGGGAGTCCAACGGCCAGCCTGAGAACAACTACAAGACCACCCCTCCT
GTGCTGGACTCCGACGGCTCCTTCTTCCTGTACTCCCGCCTGACCGTGGACAAGTCCCGCTGGCA
GGAGGGCAACGTGTTCTCCTGCTCCGTGATGCACGAGGCCCTGCACAACCACTACACCCAGAAG
TCCCTGTCCCTGTCCCTGGGCAAGTAAGAATTC
Amino acid sequence of the fusion protein BY24.23 (hCD80-Fc, IgG4)
(SEQ ID NO: 104)
METDTLLLWVLLLWVPGSTGVIHVTKEVKEVATLSCGHNVSVEELAQTRIYWQKEKKMVLTMMSG
DMNIWPEYKNRTIFDITNNLSIVILALRPSDEGTYECVVLKYEKDAFKREHLAEVTLSVKADFPTPSIS
DFEIPTSNIRRIICSTSGGFPEPHLSWLENGEELNAINTTVSQDPETEEYAVSSKLDFNMTTNHSFMCLIK
YGHLRVNQTFNWNTTKQEHFPDESKYGPPCPPCPAPEFLGGPSVFLFPPKPKDTLMISRTPEVTCVVV
DVSQEDPEVQFNWYVDGVEVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGK
The amino acid sequence ″METDTLLLWVLLLWVPGSTG″ is a signal peptide.

1.1.3 Construction of Expression Vector

The encoding nucleotide sequence of the Fc-hCD80 fusion protein is subjected to XhoI-EcoRI double enzyme digestion and linked to a glutamine synthase high-performance expression vector with a double expression box (Patent grant number: CN104195173B, obtained from Beijing Biyang Biotechnology Co., Ltd.). After a correct sequencing verification, it is used for the expression of the Fc-hCD80 fusion protein.

Similarly, the encoding nucleotide sequence of the hCD80-Fc fusion protein is subjected to XhoI-EcoRI double enzyme digestion and linked to a glutamine synthase high-performance expression vector with a double expression box (Patent grant number: CN104195173B, obtained from Beijing Biyang Biotechnology Co., Ltd.). After a correct sequencing verification, it is used for the expression of the hCD80-Fc fusion protein.

### Example 1.2. Expression of Fc-hCD80 Fusion Protein

In this example, the Fc-hCD80 fusion protein and the hCD80-Fc fusion protein as its control are expressed.

1.2.1 Transient Expression of Target Protein

293F cells (purchased from Invitrogen Corporation, catalog number: 11625-019) are suspended and cultured in a serum-free CD 293 culture solution (purchased from Invitrogen Corporation, catalog number: 11913-019). A cell culture is centrifuged prior to transfection to obtain cell pellets. The cells are suspended with a fresh serum-free CD 293 culture solution and adjusted in its cell concentration to 1×10⁶ cells/ml. A cell suspension is placed in a shake flask. Taking 100 ml of cell suspension as an example, 250 ug of a recombinant expression vector plasmid DNA containing a target gene prepared in Example 1.1 and 500 ug of polyethylenimine (PEI) (Sigma, cat: 408727) are added to 1 ml of serum-free CD 293 culture solution, are uniformly mixed and stand at room temperature for 8 minutes; and a PEI/DNA suspension is added dropwise to a shake flask containing 100 ml of cell suspension. The shake flask is mixed gently and cultured in a shaker at 5% CO₂ at 37° C. (120 rpm). A supernatant is collected and cultured after 5 days. The number of transfected cells (in volume), plasmids, and PEI can be expanded or reduced in this ratio.

1.2.2 Purification of Expression Protein

The target proteins, i.e., the Fc-hCD80 fusion protein and the hCD80-Fc fusion protein as its control, which are present in the culture supernatant collected in Example 1.2.1 above are purified with a HiTrap MabSelect SuRe 1 ml column (GE Healthcare Life Sciences product, cat: 11-0034-93) equilibrated with a pH 7.4 PBS solution. Briefly, the HiTrap MabSelect SuRe 1 ml column is equilibrated with a pH 7.4 PBS solution for 10 bed volumes at a flow rate of 0.5 ml/min; the culture supernatant collected in Example 1.2.1 above is filtered with a 0.45 m filter membrane and loaded to the HiTrap MabSelect SuRe 1 ml column equilibrated with the pH 7.4 PBS solution; after loading the supernatant, the column is first washed with the pH 7.4 PBS solution at a flow rate of 0.5 ml/min for 5-10 bed volumes and then eluted with 100 mM citrate buffer (pH 4.0) at a flow rate of 0.5 ml/min. Elution peaks are collected, and the Fc-hCD80 fusion protein and the hCD80-Fc fusion protein as its control are present in the elution peaks, respectively.

The purity and molecular weight of the target proteins BY24.30 (i.e., the Fc-hCD80 fusion protein) and BY24.23 (i.e., the hCD80-Fc fusion protein) are analyzed by SDS-PAGE electrophoresis in the presence of a reducing agent (5 mM 1,4-dithiothreitol) and staining with Coomassie blue. The results are shown in Table 10 below, where theoretical and actual measured values of molecular weight are given. Due to the glycosylation of proteins in a eukaryotic expression system, the actual measured molecular weight is higher than the theoretical predicted value.

TABLE 10
Molecular weights of purified fusion proteins

| | | | Theoretically | Actually |
|---|---|---|---|---|
| | | | predicted | measured |
| Names of | | | molecular | molecular |
| fusion | Structural | | weight | weight |
| proteins | formula | Subunit | (kDa) | (kDa) |
| BY24.30 | Fc-hCD80 | Any subunit | 50.2 | 65.0 |
| BY24.23 | hCD80-Fc | Any subunit | 50.2 | 65.0 |

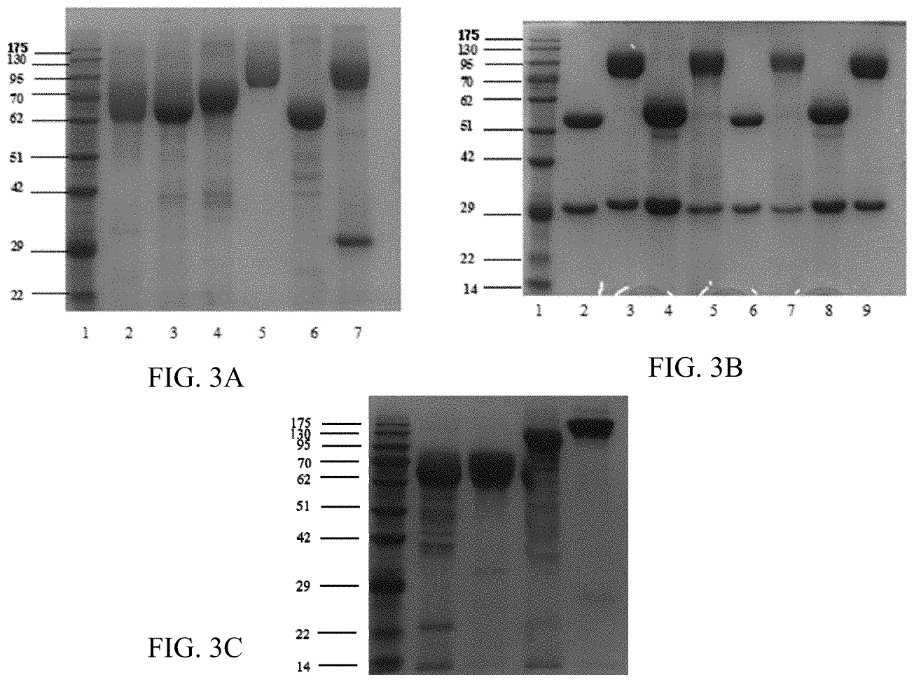

BY24.30 (i.e., the Fc-hCD80 fusion protein) and BY24.23 (i.e., the hCD80-Fc fusion protein) are subjected to SDS-PAGE electrophoresis under a reducing condition (5 mM 1,4-dithiothreitol) and a non-reducing condition, and stained with Coomassie blue. The results are shown in FIG. 3C. As can be seen from FIG. 3C, under the reducing condition, the molecular weights of BY24.30 and BY24.23 are almost exactly the same, about 65.0 kDa. However, under the non-reducing condition, the apparent molecular weights of BY24.30 and BY24.23 are significantly different, like two completely different proteins, of which the apparent molecular weight of BY24.30 is smaller, about 130 kDa, and the apparent molecular weight of BY24.23 is larger, about 175 kDa.

Since all disulfide bonds inside a polypeptide are broken under the reducing condition, peptide chains in the polypeptide are almost linear, so the protein molecular weights are usually measured under the reducing condition. Under the non-reducing condition, the disulfide bonds in the protein are well preserved, and the difference in protein electrophoresis states (apparent molecular weights) is closely related to protein conformation, in addition to being related to the molecular weight of the protein. The difference in apparent molecular weights of the fusion proteins BY24.23 and BY24.30 under the non-reducing condition indicates that BY24.23 and BY24.30 have different conformations. That is to say, the CD80 extracellular region is located at the N-terminal of Fc, and the CD80 extracellular region is located at the C-terminal of Fc, that is, the fusion proteins of two different conformations are obtained.

### Example 1.3. The Detection of the Ability of the Fc-hCD80 Fusion Protein to Specifically Bind to a Target Using ELISA

In this example, the binding ability of the Fc-hCD80 fusion protein to the target is detected. As a control, the binding ability of the hCD80-Fc fusion protein to the target is also detected. The specific method is as follows.

Recombinant human CD28 (Beijing Yigiao Shenzhou Biotechnology Co., Ltd., cat: 50103-M08H), recombinant human PD-L1 (Beijing ACROBiosystems, cat: PD1-H5229) and recombinant human CTLA-4 (Beijing Yigiao Shenzhou Biotechnology Co., Ltd., cat: 11159-H08H) are respectively diluted to 100 ng/ml and respectively coated with a 96-well ELISA plate (Corning, Inc., Item No: 42592). After coating at 37° C. for 2 h, the resulting products are washed for 3 times respectively with PBST. The washed products are closed overnight with 2% BSA PBST. On the next day, the purified fusion proteins BY24.23 (hCD80-Fc) and BY24.30 (Fc-hCD80) prepared in Example 1.2 are prepared to a concentration of 1.8 mg/ml, respectively, diluted by 3-fold gradient, with a total of 8 gradient dilutions and 2 complex wells for each concentration, and added at 50 μl/well to an ELISA plate. The fusion proteins are incubated at 37° C. for 2 h. An unbound solution is discarded and the resulting product is washed for 3 times with PBST. 1:5000 diluted goat anti-human IgG Fc-HRP (Beijing Borsi Technology Co., Ltd., cat: BHR111) is added at 50 l/well and incubated at 37° C. for 1 h. An unbound solution is discarded and the resulting product is washed for 3 times with PBST; and developed with a TMB color development solution (Kangwei Century Biotechnology Co., Ltd., cat: CW0050), and after 10 minutes, the development is terminated with 2M sulfuric acid. An absorbance OD value of each well at 450 nm is measured using an ELISA reader.

The protein concentrations of the fusion proteins BY24.23 (hCD80-Fc) and BY24.30 (Fc-hCD80) are plotted against the absorbance OD values using GraphPad Prism5 software, and data are fitted to produce a median maximum effective concentration EC₅₀ value for fusion-protein-mediated specific binding.

TABLE 11
EC₅₀ of fusion-protein-mediated specific binding

| | EC₅₀ of | EC₅₀ of | EC₅₀ of |
|---|---|---|---|
| | binding | binding | binding |
| | to CD28 | to PD-L1 | to CTLA-4 |
| Fusion proteins | (μg/ml) | (μg/ml) | (μg/ml) |
| BY24.30(Fc-hCD80) | 0.97 | 0.841 | 0.92 |
| BY24.23(hCD80-Fc) | 6.20 | 5.30 | 5.25 |

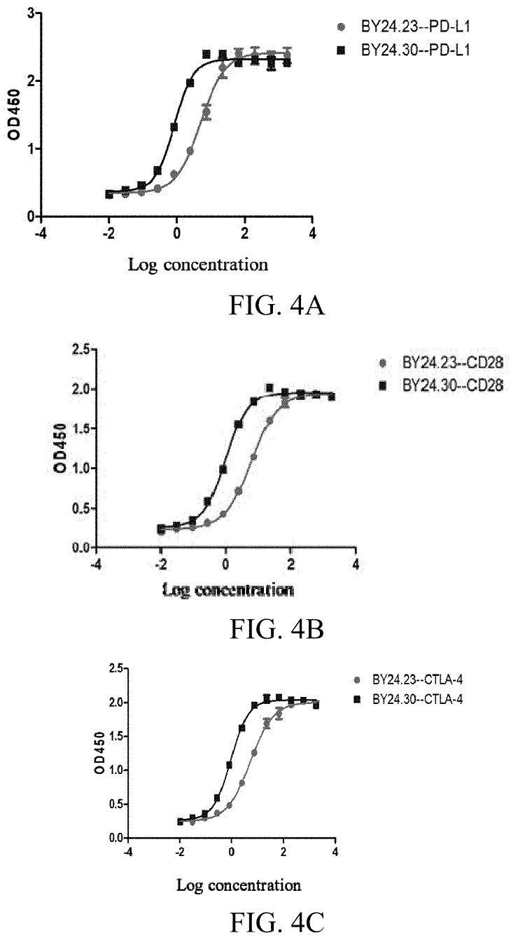

ELISA results show that the fusion proteins BY24.30 (i.e., Fc-hCD80 fusion protein) and BY24.23 (i.e., hCD80-Fc fusion protein) can both specifically bind to recombinant human PD-L1 and recombinant human CD28; and also specifically bind to recombinant human CTLA-4. FIGS. 4A, 4B and 4C show binding curves of the two fusion proteins that specifically bind to the recombinant human CD28, the recombinant human PD-L1 and the recombinant human CTLA-4, respectively. As can be seen from the drawings, when an antigen coating amount is 100 ng/ml, and the concentration of BY24.23 (hCD80-Fc) and BY24.30 (Fc-hCD80) reaches 1.8 mg/ml, the respective binding of BY24.30 (Fc-hCD80) and BY24.23 (hCD80-Fc) to CD28, CTLA-4 and PD-L1 reaches a platform, but the binding curves of these two fusion proteins to three targets are relatively visible, and the binding curves of BY24.23 (hCD80-Fc) to the targets all shift to the right compared with BY24.30 (Fc-hCD80), indicating that BY24.23 (hCD80-Fc) binds less strongly to CD28, CTLA-4 and PD-L1 than BY24.30 (Fc-hCD80).

### Example 2. Construction, Expression, Purification and Inhibition to a Mouse Tumor of a Fc-mCD80 Fusion Protein

### Example 2.1 Construction, Expression, and Purification of Expression Vector for the Fc-mCD80 Fusion Protein

According to the sequence of the CD80 extracellular domain and the Fc sequence pf mouse IgG2a, the nucleotide sequence is optimized to be suitable for expression in Chinese hamster ovary cancer cells (CHO), and Shanghai Jierui Bioengineering Co., Ltd. is entrusted to synthesize the following polynucleotide sequence of SEQ ID NO: 105. The Fc-mCD80 fusion protein produced after the expression of the nucleotide sequence is also represented herein as a fusion protein BY24.24.

Nucleotide sequence of the fusion protein BY24.24 (Fc-mCD80, IgG2a)
(SEQ ID NO: 105)
CTCGAGGCCACCATGGAGACAGACACACTCCTCCTGTGGGTGCTCCTGCTCTGGGTGCCAGGCA
GCACAGGCGAGCCTAGGGGCCCTACCATCAAGCCTTGCCCCCCATGCAAGTGCCCCGCTCCAAAC
CTCCTCGGCGGCCCTTCTGTGTTCATCTTCCCTCCAAAGATCAAGGACGTGCTGATGATCAGCCTG
TCCCCAATCGTGACATGCGTGGTGGTGGACGTGTCCGAGGACGACCCAGACGTGCAGATCTCTTG
GTTCGTGAACAACGTGGAGGTGCACACCGCTCAGACCCAGACCCACCGGGAGGACTACAACTCT
ACACTGAGAGTGGTGTCCGCCCTGCCTATCCAGCACCAGGACTGGATGTCTGGCAAGGAGTTCA
AGTGCAAGGTGAACAACAAGGACCTGCCCGCTCCTATCGAGAGAACCATCTCTAAGCCAAAGGG
CTCTGTGCGGGCTCCTCAGGTGTACGTGCTGCCTCCACCTGAGGAGGAGATGACAAAGAAGCAG
GTGACACTGACATGCATGGTGACCGACTTCATGCCAGAGGACATCTACGTGGAGTGGACCAACA
ACGGCAAGACCGAGCTGAACTACAAGAACACAGAGCCAGTGCTCGACAGCGACGGCTCTTACTT
CATGTACTCTAAGCTGAGAGTGGAGAAGAAGAACTGGGTGGAGCGGAACTCTTACTCTTGCAGC
GTGGTGCACGAGGGCCTGCACAACCACCACACCACAAAGTCTTTCTCCCGGACCCCAGGCAAGG
GAGGCGGAGGATCTGGCGGCGGAGGCAGTGGAGGCGGCGGAAGTGTGGACGAGCAGCTGTCTA
AGTCTGTGAAGGACAAGGTGCTGCTGCCTTGCCGGTACAACTCCCCTCACGAGGACGAGTCCGA
GGACCGGATCTACTGGCAGAAGCACGACAAGGTGGTGCTCAGCGTGATCGCCGGCAAGCTGAAG
GTGTGGCCCGAGTACAAGAACCGGACACTGTACGACAACACCACATACAGCCTGATCATCCTGGG
CCTCGTGCTGAGCGACCGGGGCACATACTCTTGCGTGGTGCAGAAGAAGGAGCGGGGCACATAC
GAGGTGAAGCACCTCGCCCTCGTGAAGCTGTCTATCAAGGCCGACTTCTCTACCCCTAACATCAC
AGAGTCTGGCAACCCTAGCGCCGACACAAAGCGGATCACATGCTTCGCTAGCGGCGGCTTCCCTA
AGCCACGGTTCTCTTGGTTGGAAAACGGCCGGGAGCTGCCAGGCATCAACACCACCATCTCTCA
GGACCCAGAGTCCGAGCTGTACACAATCTCTAGCCAGCTCGACTTCAACACCACCCGGAACCAC
ACAATCAAGTGCCTCATCAAGTACGGCGACGCTCACGTGTCTGAGGACTTCACATGGGAGAAGC
CACCTGAGGACCCTCCCGACTCTAAGAACTAAGAATTC
Amino acid sequence of the fusion protein BY24.24 (Fc-mCD80, IgG2a)
(SEQ ID NO: 106)
METDTLLLWVLLLWVPGSTGEPRGPTIKPCPPCKCPAPNLLGGPSVFIFPPKIKDVLMISLSPIVTCVVV
DVSEDDPDVQISWFVNNVEVHTAQTQTHREDYNSTLRVVSALPIQHQDWMSGKEFKCKVNNKDLP
APIERTISKPKGSVRAPQVYVLPPPEEEMTKKQVTLTCMVTDFMPEDIYVEWTNNGKTELNYKNTEP
VLDSDGSYFMYSKLRVEKKNWVERNSYSCSVVHEGLHNHHTTKSFSRTPGKGGGGSGGGGSGGGG
SVDEQLSKSVKDKVLLPCRYNSPHEDESEDRIYWQKHDKVVLSVIAGKLKVWPEYKNRTEYDNTTY
SLIILGLVLSDRGTYSCVVQKKERGTYEVKHLALVKLSIKADFSTPNITESGNPSADTKRITCFASGGFP
KPRFSWLENGRELPGINTTISQDPESELYTISSQLDFNTTRNHTIKCLIKYGDAHVSEDFTWEKPPEDPP
DSKN
The amino acid sequence ″METDTLLLWVLLLWVPGSTG″ is a signal peptide.

The encoding nucleotide sequence of BY24.24 is subjected to XhoI double enzyme digestion and linked to a glutamine synthase high-performance expression vector with a double expression box (Patent grant number: CN104195173B, obtained from Beijing Biyang Biotechnology Co., Ltd.). After a correct sequencing verification, the recombinant vector is used for the expression of the Fc-mCD80 fusion protein. The expressed Fc-mCD80 fusion protein is also referred to as a fusion protein BY24.24.

Similar to Example 1.2 above, the expression and purification of the Fc-mCD80 fusion protein are performed, and the molecular weights are measured. The results are shown in Table 12 below, where theoretical and actual measured values of molecular weight are given. Due to the glycosylation of proteins in a eukaryotic expression system, the actual measured molecular weight is higher than the theoretical predicted value.

TABLE 12
Molecular weights of purified fusion proteins

| | | | Theoretical | Actual |
|---|---|---|---|---|
| | | | predicted | measured |
| Names of | | | molecular | molecular |
| fusion | Structural | | weight | weight |
| proteins | formula | Subunit | (kDa) | (kDa) |
| BY24.24 | Fc-mCD80 | Any subunit | 50.0 | 65.0 |

### Example 2.2. Inhibitory Effect of Fusion Protein BY24.24 (Fc-mCD80) on Mouse Tumors

An anti-tumor effect of the fusion protein BY24.24 is preliminarily evaluated through a BALB/c mouse subcutaneous tumor model of a mouse colon cancer cell CT-26, which provides data support for subsequent preclinical pharmacodynamic tests.

Test Method

The BALB/c mouse subcutaneous tumor model of the mouse colon cancer cell CT-26 is established, and 18 qualified tumorigenic animals are screened and randomly divided into 3 groups: group 1 (PBS), group 2 (fusion protein BY24.24, 0.7 mg/kg), group 3 (mPD1, Bio X Cell company product, anti-mouse PD1 antibody, clone number: RMP1-14, 1.0 mg/kg), 6 mice in each group. The animals are administered by intraperitoneal injection, with an administration volume of 10 ml/kg, once every 3 days, for 6 consecutive administrations. The animals are euthanized on the 19^{th} day. During the administration period, the general clinical symptoms of the animals are observed twice a day, and the body weights and tumor diameters are measured every 3 days. After euthanasia, the tumors are excised, weighed, and photographed. It is effective that the relative tumor proliferation rate T/C %≤40% and P≤0.05 as compared to a negative control group against the relative tumor volume RTV.

Experimental Results

During the whole experiment, no animals die. The body weights of the animals increase slightly, but there is no significant difference in body weight between groups (P>0.05).

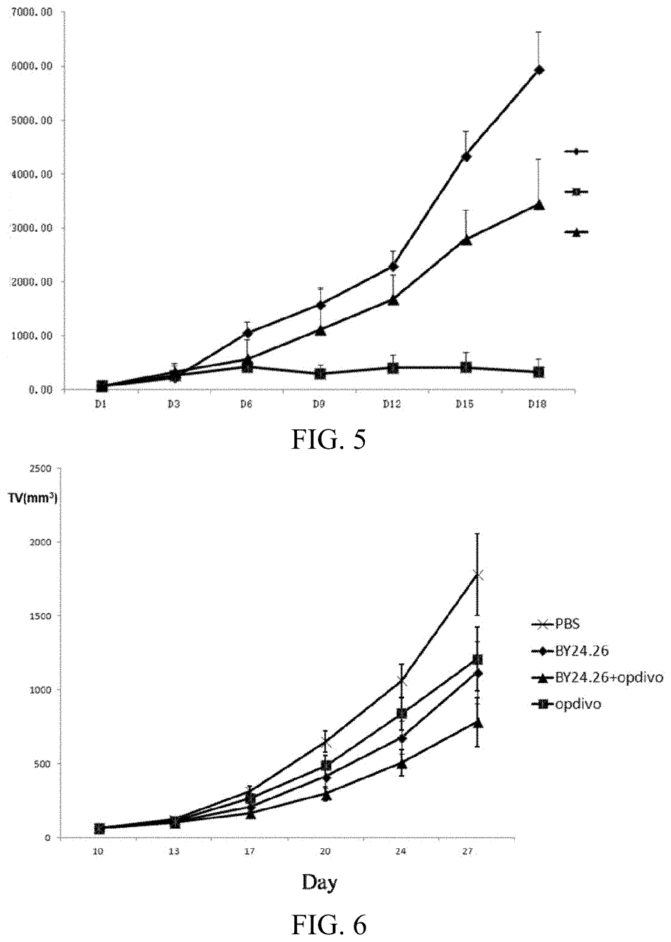

The tumor growth in each group is as follows: on the 19^{th} day after the first administration, in the PBS group, the average tumor volume is 5931.22±702.88 mm³, and the RTV is 84.01±21.23; in the BY24.24 group, the average tumor volume is 327.63±241.33 mm³ and the RTV is 3.16±5.20; and in the mPD1 group, the average tumor volume is 3437.04±846.53 mm³, and the RTV is 15.74±12.05. Compared with the mPD1 group and the PBS group, the BY24.24 group has significantly lower mean tumor volume and RTV values, has a statistical meaning (P<0.001 vs. PBS group; P<0.01 vs. mPD1 group) and also has a significant anti-tumor effect. mPD1 has an effect of inhibiting tumor proliferation, but is not statistically different from the PBS group (P>0.05) (from FIG. 5).

Conclusion:

Under the same molar dose, BY24.24 (fusion protein Fc-mCD80) has a significantly better growth inhibitory effect on mouse colon cancer cell CT26 than mPD1 (anti-mouse PD1 antibody).

### Example 3. Construction and Functional Study of an IgG1 Fc-CD80 Fusion Protein Conjugate Having an Anti-VEGF Antibody Fab

Example 3.1 Construction, Expression and Purification of an IgG1 Fc-CD80 Fusion Protein Conjugate Having an Anti-VEGF Antibody Fab

According to an amino acid sequence of an anti-VEGF monoclonal antibody bevacizumab numbered 8017 in the International Nonproprietary Name (INN) database, the following nucleotide sequences are optimized to be suitable for expression in Chinese hamster ovary cancer cells (CHO), and Shanghai Jierui Bioengineering Co., Ltd. is entrusted to synthesize a nucleotide sequence together with the sequence of the CD80 extracellular domain in Table 1. The resulting conjugate after expression of the nucleotide sequence is denoted herein as a conjugate BY24.26 (bevacizumab-CD80).

Light-chain (BY24.26L) nucleotide sequence of BY24.26 (bevacizumab-CD80)
(SEQ ID NO: 107)
CTCGAGGCCACCATGGAGACAGATACTCTGCTGCTCTGGGTCCTGCTGCTGTGGGTGCCAGGCAG
CACAGGCGACATCCAGATGACCCAGTCCCCTTCCTCCCTGTCTGCCTCCGTGGGCGACAGGGTGA
CCATCACCTGTTCCGCATCCCAGGATATCTCCAACTACCTGAACTGGTATCAGCAGAAGCCAGGA
AAGGCCCCCAAGGTGCTGATCTACTTTACTTCTTCCCTGCATTCCGGCGTGCCCTCCAGGTTCTCT
GGCTCCGGGTCTGGAACCGACTTTACACTGACAATCTCCTCTCTGCAGCCAGAAGACTTTGCCAC
CTATTACTGCCAGCAGTACTCCACTGTCCCCTGGACCTTCGGGCAAGGAACCAAAGTGGAAATCA
AGAGGACCGTGGCCGCCCCCAGCGTGTTCATCTTCCCTCCCAGCGACGAGCAGCTGAAGTCTGG
CACCGCCAGCGTGGTGTGCCTGCTGAACAACTTCTACCCCCGCGAGGCCAAGGTGCAGTGGAAG
GTGGACAACGCCCTGCAGAGCGGCAACAGCCAGGAGAGCGTGACCGAGCAGGACTCCAAGGAC
AGCACCTACAGCCTGAGCAGCACCCTGACCCTGAGCAAGGCCGACTACGAGAAGCACAAGGTGT
ACGCCTGCGAGGTGACCCACCAGGGACTGTCTAGCCCCGTGACCAAGAGCTTCAACCGGGGCGA
GTGCTAAGAATTC
Light-chain (BY24.26L) amino acid sequence of a conjugate BY24.26
(bevacizumab-CD80)
(SEQ ID NO: 108)
METDTLLLWVLLLWVPGSTGDIQMTQSPSSLSASVGDRVTITCSASQDISNYLNWYQQKPGKAPKVL
IYFTSSLHSGVPSRFSGSGSGTDFTLTISSLQPEDFATYYCQQYSTVPWTFGQGTKVEIKRTVAAPSVFIF
PPSDEQLKSGTASVVCLLNNFYPREAKVQWKVDNALQSGNSQESVTEQDSKDSTYSLSSTLTLSKAD
YEKHKVYACEVTHQGLSSPVTKSFNRGEC
Heavy-chain nucleotide sequence of a conjugate BY24.26 (bevacizumab-CD80)
(SEQ ID NO: 109)
TCTAGAGCCACCATGGAAACCGACACACTGCTGCTGTGGGTCCTGCTGCTGTGGGTGCCAGGCTC
TACAGGCGAAGTGCAGCTGGTCGAATCTGGCGGCGGACTGGTGCAACCAGGGGGCAGCCTGAG
GCTGTCCTGCGCCGCTTCTGGATACACCTTTACCAATTACGGCATGAATTGGGTGAGGCAGGCTCC
TGGCAAGGGCCTGGAGTGGGTGGGATGGATCAACACCTACACCGGCGAGCCCACCTACGCCGCT
GACTTTAAGAGGAGGTTCACCTTCTCTCTGGACACCTCCAAGTCCACCGCTTACCTGCAAATGAA
CTCTCTGAGGGCTGAAGACACCGCCGTGTACTACTGCGCCAAGTACCCCCATTACTACGGGTCCT
CCCACTGGTACTTCGACGTGTGGGGCCAAGGCACTCTGGTCACCGTGAGCTCCGCCAGCACCAA
GGGCCCCAGCGTCTTCCCACTGGCTCCTTCCTCTAAAAGCACTAGCGGAGGGACCGCAGCCCTGG
GCTGTCTGGTGAAAGACTACTTCCCCGAGCCCGTGACCGTCTCCTGGAACTCTGGAGCCCTGACC
TCCGGGGTGCACACCTTTCCCGCCGTGCTGCAGTCTTCTGGACTGTACTCCCTGTCCTCCGTCGTG
ACTGTGCCCAGCTCCTCCCTGGGAACTCAGACATACATCTGCAACGTGAACCACAAGCCTTCCAA
CACAAAGGTGGACAAGAAAGTCGAGCCCAAGAGCTGTGATAAGACCCATACATGTCCCCCATGC
CCCGCTCCAGAACTGCTGGGCGGACCTTCCGTGTTTCTGTTCCCACCCAAACCAAAGGACACACT
GATGATCAGCAGAACCCCTGAGGTGACTTGCGTGGTCGTGGACGTGAGCCATGAGGACCCCGAG
GTGAAGTTCAACTGGTATGTGGATGGCGTGGAAGTGCATAATGCCAAGACAAAACCTAGGGAAG
AGCAGTACAACAGCACCTACAGGGTGGTGAGCGTGCTGACCGTGCTGCACCAGGATTGGCTGAA
CGGCAAGGAATACAAGTGCAAGGTGTCCAATAAGGCTCTGCCTGCACCTATCGAGAAGACCATCA
GCAAAGCCAAGGGCCAACCCAGAGAGCCTCAAGTCTACACCCTGCCCCCAAGCAGGGAGGAGA
TGACCAAAAATCAAGTGAGCCTGACATGCCTGGTCAAAGGCTTCTACCCTAGCGACATCGCCGTG
GAGTGGGAGAGCAATGGCCAGCCTGAGAACAACTACAAGACCACTCCCCCCGTCCTGGATAGCG
ACGGCAGCTTCTTCCTGTACTCCAAACTGACAGTCGATAAAAGCAGGTGGCAGCAAGGCAATGTC
TTTAGCTGTAGCGTGATGCACGAGGCCCTGCATAACCACTACACTCAAAAGTCCCTGTCCCTGAG
CCCCGGAAAGGGAGGCGGAGGATCTGGCGGCGGAGGCAGTGGAGGCGGCGGAAGTGTGATCCA
TGTAACAAAAGAAGTGAAGGAAGTGGCTACACTCTCTTGCGGCCACAACGTGTCCGTGGAGGAA
CTAGCTCAGACCCGGATCTATTGGCAGAAAGAAAAGAAGATGGTGCTGACCATGATGTCCGGCGA
CATGAACATTTGGCCAGAGTACAAGAACCGCACAATTTTCGACATTACAAACAACCTCTCTATTGT
GATTCTGGCTCTCAGGCCTAGCGACGAGGGCACATACGAGTGCGTGGTGCTCAAGTACGAGAAG
GACGCTTTCAAGCGGGAGCACCTCGCTGAGGTGACCCTGTCCGTGAAGGCCGACTTCCCTACTCC
ATCTATCTCCGACTTCGAGATCCCTACCTCTAACATCAGGCGGATCATCTGCAGCACCTCCGGCGG
CTTCCCAGAGCCTCACCTCTCTTGGCTGGAGAACGGCGAGGAGCTGAACGCTATCAACACCACA
GTGTCTCAGGACCCTGAGACCGAGCTGTACGCCGTGTCCTCTAAGCTCGACTTCAACATGACCAC
AAACCACTCCTTCATGTGCCTGATCAAGTACGGCCACCTGAGAGTGAACCAGACATTCAACTGGA
ACACAACCAAGCAGGAGCACTTCCCATAAGTCGAC
Heavy-chain amino acid sequence of a conjugate BY24.26 (bevacizumab-CD80)
(SEQ ID NO: 110)
METDTLLLWVLLLWVPGSTGEVQLVESGGGLVQPGGSLRLSCAASGYTFTNYGMNWVRQAPGKGL
EWVGWINTYTGEPTYAADFKRRFTFSLDTSKSTAYLQMNSLRAEDTAVYYCAKYPHYYGSSHWYFD
VWGQGTLVTVSSASTKGPSVFPLAPSSKSTSGGTAALGCLVKDYFPEPVTVSWNSGALTSGVHTFPAV
LQSSGEYSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQKSL
SLSPGKGGGGSGGGGSGGGGSVIHVTKEVKEVATLSCGHNVSVEELAQTRIYWQKEKKMVLTMMS
GDMNIWPEYKNRTIFDITNNLSIVILALRPSDEGTYECVVLKYEKDAFKREHLAEVTLSVKADFPTPSI
SDFEIPTSNIRRIICSTSGGFPEPHLSWLENGEELNAINTTVSQDPETELYAVSSKLDFNMTTNHSFMCLI
KYGHLRVNQTFNWNTTKQEHFP
The underlined part ″METDTLLLWVLLLWVVPGG″ is a signal peptide sequence.

Shanghai Jierii Bioengineering Co., Ltd. synthesizes light-chain (XhoI-EcoRI) and heavy-chain encoding nucleotide sequences of the above conjugate BY24.26. The light chain and the heavy chain in the encoding nucleotide sequence of the BY24.26 are subjected to XhoI-EcoRI double enzyme digestion and XhoI-SalI double enzyme digestion respectively and then linked to a glutamine synthase high-performance expression vector with a double expression box (Patent grant number: CN104195173B, obtained from Beijing Biyang Biotechnology Co., Ltd.). After a correct sequencing verification, an expression vector of the conjugate BY24.26 is obtained for expression.

Similar to the above example 1.2, 250 ug of the prepared recombinant expression vector plasmid DNA including light-chain and heavy-chain nucleotide sequences of the conjugate BY24.26 and 500 ug of polyethylenimine (PEI) (Sigma, cat: 408727) are added to 1 ml of serum-free CD 293 culture solution, are uniformly mixed and stand at room temperature for 8 minutes; and a PEI/DNA suspension is added dropwise to a shake flask containing 100 ml of cell suspension. The shake flask is mixed gently and cultured in a shaker at 5% CO₂ at 37° C. (120 rpm). A supernatant is collected and cultured after 5 days.

The expression and purification of the conjugate BY24.26 are performed and the molecular weights are measured. The results are shown in Table 13 below, where theoretical and actual measured values of molecular weight are given. Due to the glycosylation of proteins in a eukaryotic expression system, the actual measured molecular weight is higher than the theoretical predicted value.

TABLE 13
Molecular weights of purified proteins

| | | Theoretical | Actual |
|---|---|---|---|
| | | predicted | measured |
| | | molecular | molecular |
| | | weight | weight |
| Names of proteins | Subunit | (kDa) | (kDa) |
| Control antibody | Light chain | 23.4 | 25 |
| bevacizumab | Heavy chain | 49.8 | 50 |
| Conjugate | Light chain | 23.4 | 25 |
| BY24.26 | Heavy chain | 74.4 | 95.0 |

### Example 3.2 Using Biacore T100 to Determine the Effect of the Conjugate BY24.26 on the Binding Ability of a Second Target

Surface plasmon resonance measurement is performed at 25° C. on a BIAcore® T100 instrument (GE Healthcare Biosciences AB, Sweden). The binding ability of an antigen VEGF165 (Beijing Yigiao Shenzhou Biotechnology Co., Ltd., catr: 11066-HNAH) to the conjugate BY24.26 is determined by surface plasmon resonance at 25° C. using HBS-EP (10 mM HEPES, pH 7.4, 150 mM NaCl, 3 mM EDTA, and 0.005% surfactant P20). Briefly, an anti-IgG antibody (GE Healthcare Life Sciences, cat: BR-1008-39) is immobilized directly onto a CM5 chip using a standard amine coupling kit, according to the manufacturer's instructions. The purified conjugate BY24.26 or the control antibody bevacizumab (purchased from Roche) is diluted in HEPES-buffered saline, specifically binds to chip-conjugated sheep anti-human IgG, and is injected at a flow rate of 5 l/min on a reaction matrix. A rate constant is obtained by kinetic binding measurements at different antigen concentrations within a range of 1.25 nM to 1000 nM. Based on kinetic information provided by the binding process and dissociation process in a sensing diagram, Biacore can detect a binding rate ka(1/ms), dissociation rate kd(1/s) and affinity K_{D}(M) of a ligand to an analyte. Data analysis is performed using BIA evaluation software (BIAevaluation 4.1 software, from GE Healthcare Biosciences AB, Sweden) to obtain affinity data in Table 14.

TABLE 14
Binding of a conjugate to a second target

| Names of proteins | Target | ka (1/Ms) | kd (1/s) | K_{D} (M) |
|---|---|---|---|---|
| Control antibody | VEGF-A | 3.52E+5 | 2.42E−5 | 5.87E−11 |
| bevacizumab | | | | |
| Conjugate | | 6.42E+5 | 3.23E−5 | 5.03E−11 |
| BY24.26 | | | | |

As can be seen from Table 14, the binding ability of the conjugate BY24.26 to the second target, VEGF-A, is comparable to that of bevacizumab.

### Example 3.3 In Vivo Biological Activity Study of Conjugate BY24.26 that can Bind to CD80 Ligand and VEGF

This embodiment investigates a tumor suppression effect and safety of the conjugate BY24.26, and discusses a synergistic effect and mechanism of the combination of the conjugate BY24.26 and a PD1 antibody opdivo.

Test Method

Human hepatoma cells HUH7 (purchased from Proton (Beijing) Biotechnology Co., Ltd.) are inoculated subcutaneously to right anterior lateral thorax ribs of male NCG mice (purchased from Jiangsu Jihui Yaokang Biotechnology Co., Ltd.); PBMC cells are inoculated in a tibia marrow cavity of each mouse, and the mice are administered in groups when the tumor grows to about 65 mm³, a total of 4 groups, 6 mice in each group, respectively, i.e., a solvent (PBS) group, an opdivo group (purchased from Bristol-Myers Squibb, 10 mg/kg, ip, q3d×6), a conjugate BY24.26 group (13 mg/kg, ip, q3d×6) and an opdivo (10 mg/kg, ip, q3d×6)+BY24.26 (13 mg/kg, IP, Q3D×6) group; and each group is administrated equimolarly according to molecular weights. The tumor volumes and body weights are measured weekly, and a relationship between the changes in weight and tumor volume of tumor-bearing mice and the administration time is recorded. At the end of the experiment, the tumor-bearing mice are euthanized, the tumors are excised, weighed, photographed, and the expression of markers CD4, CD8 and CD31 is detected by immunohistochemistry (IHC) after tumors in each group are immobilized. A T/C value is calculated according to the tumor volume, and the calculation formula is as follows: a tumor volume ratio of the treatment group/control group T/C (%)=RTV of the treatment group/RTV of the control group×100%. An average RTV of tumor volumes in the treatment group (T)/control group (C) is a ratio of tumor volumes after administration and prior to administration. Tumor growth inhibition rate (%)=(1−T/C)×100%.

Results:
The effects of administration on the safety of mice:

during the treatment period, the mice in each group are ingested with food and water normally, and no abnormal behaviors appear. The mice generally exhibit good conditions; and no mice die.
Biological activities of Opdivo, conjugate BY24.26 and Opdivo+BY24.26 (bevacizumab-CD80) groups: the tumor volumes in the solvent group, Opdivo group, conjugate BY24.26 group and Opdivo+BY24.26 group are 1779±275 mm³, 1209±216 mm³, 1116±209 mm³ and 780±163 mm³, respectively. The tumor growth inhibition rates in the Opdivo group, conjugate BY24.26 group and Opdivo+BY24.26 group are 32%, 40% and 58%, respectively, and the tumor volumes of these three groups are significantly lower than that in the solvent group (p<0.05); and the tumor volume in the Opdivo+BY24.26 group is significantly lower than that in the conjugate BY24.26 group and the Opdivo group (p<0.05). It can be seen that the conjugate BY24.26 has a synergistic effect with Opdivo.

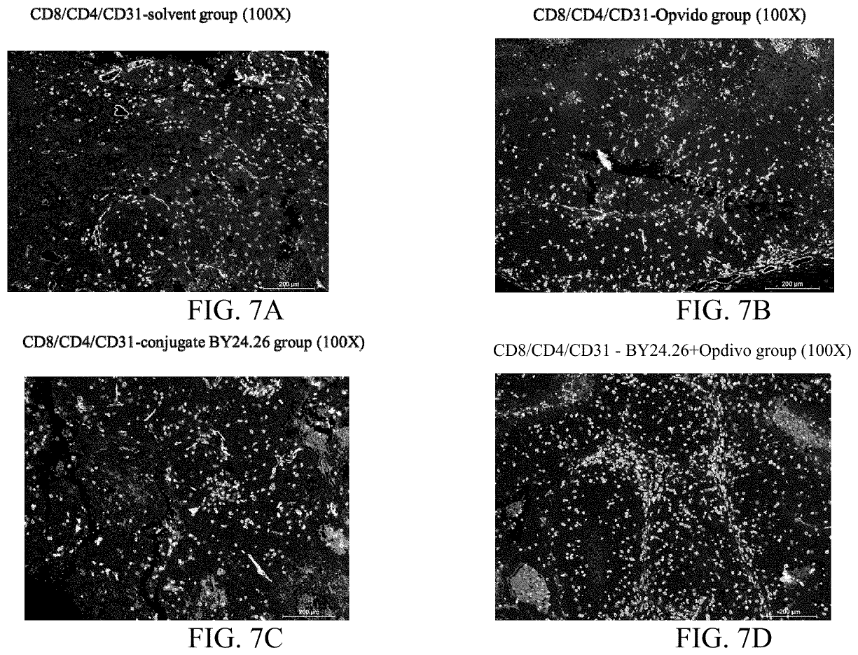

At the end of the experiment, the expressions of markers CD8, CD4 and CD31 in each group are detected by immunohistochemistry (IHC). The results are shown in FIG. 7A, FIG. 7B, FIG. 7C, and FIG. 7D, respectively (Note: in each drawing, green represents a positive expression site of the marker CD8, red represents a positive expression site of the marker CD4, yellow represents a positive expression site of the marker CD31, and blue represents a nucleus). In the solvent group, the percentage of CD8⁺ tumor-infiltrating lymphocytes is 6.9%, the percentage of CD4⁺ tumor-infiltrating lymphocytes is 3.3%, and the average number of CD31⁺ blood vessels is 29; in the Opdivo group, CD8⁺, CD4⁺ and CD31⁺ blood vessels are 24.5%, 11.1%, and 18, respectively; in the conjugate BY24.26 group, CD8⁺, CD4⁺ and CD31⁺ blood vessels are 25.3%, 21.9%, and 11, respectively; and in the Opdivo+BY24.26 group, CD8⁺, CD4⁺ and CD31⁺ blood vessels are 40.1%, 22.3%, and 18, respectively. The detection results of IHC show that after the treatment of the conjugate BY24.26 alone, the numbers of CD8+ and CD4⁺ tumor-infiltrating lymphocytes increase significantly compared with the solvent group, while the number of CD31⁺ blood vessels decreases significantly; after the combined administration of BY24.26 and Opdivo, the number of CD8⁺ tumor-infiltrating lymphocytes increases significantly compared with the solvent group, and greatly increases compared with the single administration of the conjugate BY24.26 group and the Opdivo group.

Conclusion:

The individual administration of the conjugate BY24.26 and the combined administration of the conjugate BY24.26 and the PD1 antibody Opdivo both produce an anti-tumor effect on a human liver cancer model HUH7, but the anti-tumor effect of combined administration is better than that of the corresponding individual administration group. IHC detection shows that the anti-tumor effect of the conjugate BY24.26 is closely related to recruitment of T cells to infiltrate the interior of the tumor and inhibition of tumor neovascularization, and the anti-tumor effect of the conjugate BY24.26 combined with the PD1 antibody Opdivo is related to their synergistic promotion of CD8+T lymphocyte infiltration to the interior of the tumor. Throughout the experiment, the animals in each group have favorable tolerance on the administration, indicating that the conjugate BY24.26 is safe.

### Example 4. Construction and Functional Study of an IgG1 Fc-CD80 Fusion Protein Conjugate Having an Anti-HER2 Antibody Fab

### Example 4.1 Construction, Expression and Purification of an IgG1 Fc-CD80 Fusion Protein Conjugate Having an Anti-HER2 Antibody Fab

According to an amino acid sequence of an anti-HER2 monoclonal antibody trastuzumab numbered 7637 in the International Nonproprietary Name (INN) database, the following nucleotide sequences are optimized to be suitable for expression in Chinese hamster ovary cancer cells (CHO), and Shanghai Jierui Bioengineering Co., Ltd. is enstrusted to synthesize a nucleotide sequence together with the sequence of the CD80 extracellular domain in Table 1. The resulting conjugate after expression of the nucleotide sequence is denoted herein as a conjugate BY12.7 (trastuzumab-CD80).

Light-chain (BY12.7L) amino acid sequence of the conjugate BY12.7:
(SEQ ID NO: 111)
CTCGAGGCCACCATGGAGACCGATACTCTGCTGCTCTGGGTGCTGCTGCTGTGGGTCCCCGGCTC
CACCGGCGACATCCAGATGACCCAATCCCCCTCCTCTCTGTCTGCCTCCGTGGGAGATAGGGTGA
CCATCACATGCAGGGCATCTCAGGACGTCAACACCGCTGTCGCTTGGTACCAGCAAAAGCCCGG
AAAGGCCCCCAAGCTGCTGATTTACAGCGCTTCCTTTCTGTATTCCGGCGTGCCCTCCAGGTTTTC
TGGAAGCAGGAGCGGGACCGATTTTACCCTGACAATCTCCAGCCTGCAACCCGAGGACTTCGCC
ACCTACTATTGCCAGCAGCACTACACCACACCCCCTACATTCGGCCAAGGCACCAAGGTGGAGAT
CAAGAGGACCGTGGCCGCCCCCAGCGTGTTCATCTTCCCTCCCAGCGACGAGCAGCTGAAGTCT
GGCACCGCCAGCGTGGTGTGCCTGCTGAACAACTTCTACCCCCGCGAGGCCAAGGTGCAGTGGA
AGGTGGACAACGCCCTGCAGAGCGGCAACAGCCAGGAGAGCGTGACCGAGCAGGACTCCAAGG
ACAGCACCTACAGCCTGAGCAGCACCCTGACCCTGAGCAAGGCCGACTACGAGAAGCACAAGGT
GTACGCCTGCGAGGTGACCCACCAGGGACTGTCTAGCCCCGTGACCAAGAGCTTCAACCGGGGC
GAGTGCTAAGAATTC
Light-chain (BY12.7L) amino acid sequence of a conjugate BY12.7:
(SEQ ID NO: 112)
METDTLLLWVLLLWVPGSTGDIQMTQSPSSLSASVGDRVTITCSASQDISNYLNWYQQKPGKAPKVL
IYFTSSLHSGVPSRFSGSGSGTDFTLTISSLQPEDFATYYCQQYSTVPWTFGQGTKVEIKRTVAAPSVFIF
PPSDEQLKSGTASVVCLLNNFYPREAKVQWKVDNALQSGNSQESVTEQDSKDSTYSLSSTLTLSKAD
YEKHKVYACEVTHQGLSSPVTKSFNRGEC
Heavy-chain nucleotide sequence of a conjugate BY12.7
(SEQ ID NO: 113)
TCTAGAGCCACCATGGAAACCGACACACTGCTGCTGTGGGTCCTGCTGCTGTGGGTGCCAGGCTC
TACAGGCGAGGTCCAACTGGTGGAGTCCGGAGGGGGACTGGTGCAACCTGGCGGCAGCCTGAG
GCTGTCCTGCGCTGCCTCCGGCTTCAACATCAAGGACACCTACATTCACTGGGTGAGGCAAGCAC
CCGGAAAGGGCCTGGAGTGGGTGGCCAGGATCTACCCCACCAACGGCTACACCAGGTACGCCGA
CTCCGTGAAGGGCAGGTTCACCATCTCCGCCGATACCTCCAAGAACACCGCCTACCTGCAAATGA
ACTCCCTGAGGGCCGAAGACACAGCCGTGTACTACTGCTCCAGGTGGGGAGGGGACGGCTTCTA
CGCCATGGACTATTGGGGCCAAGGCACTCTGGTCACCGTGAGCTCCGCCAGCACCAAGGGCCCC
AGCGTCTTCCCACTGGCTCCTTCCTCTAAAAGCACTAGCGGAGGGACCGCAGCCCTGGGCTGTCT
GGTGAAAGACTACTTCCCCGAGCCCGTGACCGTCTCCTGGAACTCTGGAGCCCTGACCTCCGGG
GTGCACACCTTTCCCGCCGTGCTGCAGTCTTCTGGACTGTACTCCCTGTCCTCCGTCGTGACTGTG
CCCAGCTCCTCCCTGGGAACTCAGACATACATCTGCAACGTGAACCACAAGCCTTCCAACACAAA
GGTGGACAAGAAAGTCGAGCCCAAGAGCTGTGATAAGACCCATACATGTCCCCCATGCCCCGCTC
CAGAACTGCTGGGCGGACCTTCCGTGTTTCTGTTCCCACCCAAACCAAAGGACACACTGATGATC
AGCAGAACCCCTGAGGTGACTTGCGTGGTCGTGGACGTGAGCCATGAGGACCCCGAGGTGAAGT
TCAACTGGTATGTGGATGGCGTGGAAGTGCATAATGCCAAGACAAAACCTAGGGAAGAGCAGTA
CAACAGCACCTACAGGGTGGTGAGCGTGCTGACCGTGCTGCACCAGGATTGGCTGAACGGCAAG
GAATACAAGTGCAAGGTGTCCAATAAGGCTCTGCCTGCACCTATCGAGAAGACCATCAGCAAAGC
CAAGGGCCAACCCAGAGAGCCTCAAGTCTACACCCTGCCCCCAAGCAGGGAGGAGATGACCAA
AAATCAAGTGAGCCTGACATGCCTGGTCAAAGGCTTCTACCCTAGCGACATCGCCGTGGAGTGGG
AGAGCAATGGCCAGCCTGAGAACAACTACAAGACCACTCCCCCCGTCCTGGATAGCGACGGCAG
CTTCTTCCTGTACTCCAAACTGACAGTCGATAAAAGCAGGTGGCAGCAAGGCAATGTCTTTAGCT
GTAGCGTGATGCACGAGGCCCTGCATAACCACTACACTCAAAAGTCCCTGTCCCTGAGCCCCGGA
AAGGGAGGCGGAGGATCTGGCGGCGGAGGCAGTGGAGGCGGCGGAAGTGTGATCCATGTAACA
AAAGAAGTGAAGGAAGTGGCTACACTCTCTTGCGGCCACAACGTGTCCGTGGAGGAACTAGCTC
AGACCCGGATCTATTGGCAGAAAGAAAAGAAGATGGTGCTGACCATGATGTCCGGCGACATGAA
CATTTGGCCAGAGTACAAGAACCGCACAATTTTCGACATTACAAACAACCTCTCTATTGTGATTCT
GGCTCTCAGGCCTAGCGACGAGGGCACATACGAGTGCGTGGTGCTCAAGTACGAGAAGGACGCT
TTCAAGCGGGAGCACCTCGCTGAGGTGACCCTGTCCGTGAAGGCCGACTTCCCTACTCCATCTAT
CTCCGACTTCGAGATCCCTACCTCTAACATCAGGCGGATCATCTGCAGCACCTCCGGCGGCTTCCC
AGAGCCTCACCTCTCTTGGCTGGAGAACGGCGAGGAGCTGAACGCTATCAACACCACAGTGTCT
CAGGACCCTGAGACCGAGCTGTACGCCGTGTCCTCTAAGCTCGACTTCAACATGACCACAAACCA
CTCCTTCATGTGCCTGATCAAGTACGGCCACCTGAGAGTGAACCAGACATTCAACTGGAACACAA
CCAAGCAGGAGCACTTCCCATAAGTCGAC
Heavy-chain (BY12.7H) amino acid sequence of a conjugate BY12.7
(SEQ ID NO: 114)
METDTLLLWVLLLWVPGSTGEVQLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGGDGFYAMDYWG
QGTLVTVSSASTKGPSVFPLAPSSKSTSGGTAALGCEVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPG
KGGGGSGGGGSGGGGSVIHVTKEVKEVATLSCGHNVSVEELAQTRIYWQKEKKMVLTMMSGDMNI
WPEYKNRTIFDITNNLSIVILALRPSDEGTYECVVLKYEKDAFKREHLAEVTLSVKADFPTPSISDFEIP
TSNIRRIICSTSGGFPEPHLSWLENGEELNAINTTVSQDPETELYAVSSKLDFNMTTNHSFMCLIKYGHL
RVNQTFNWNTTKQEHFP
The underlined part ″METDTLLLWVLLLWVVPGG″ is a signal peptide sequence.

Shanghai Jierii Bioengineering Co., Ltd. synthesizes light-chain and heavy-chain encoding nucleotide sequences of the above conjugate BY12.7. The light chain and the heavy chain in the encoding nucleotide sequence of the BY12.7 are subjected to XhoI-EcoRI double enzyme digestion and XhoI-SalI double enzyme digestion respectively and then linked to a glutamine synthase high-performance expression vector with a double expression box (Patent grant number: CN104195173B, obtained from Beijing Biyang Biotechnology Co., Ltd.). After a correct sequencing verification, an expression vector of the conjugate BY12.7 is obtained for expression.

Similar to Example 3.1 above, the expression and purification of the conjugate BY12.7 are performed, and the molecular weights are measured. The results are shown in Table 15 below, where theoretical and actual measured values of molecular weight are given. Due to the glycosylation of proteins in a eukaryotic expression system, the actual measured molecular weight is higher than the theoretical predicted value

TABLE 15
Molecular weights of purified proteins

| | | Theoretical | Actual |
|---|---|---|---|
| | | predicted | measured |
| | | molecular | molecular |
| | | weight | weight |
| Names of proteins | Subunit | (kDa) | (kDa) |
| Conjugate BY12.7 | Light chain | 23.4 | 25 |
| | Heavy chain | 73.7 | 95.0 |
| Control antibody | Light chain | 23.4 | 25 |
| trastuzumab | Heavy chain | 49.2 | 50 |

### Example 4.2 Using Biacore T100 to Determine the Effect of the Conjugate BY12.7 on the Binding Ability to a Second Target

Surface plasmon resonance measurement is performed at 25° C. on a BIAcore® T100 instrument (GE Healthcare Biosciences AB, Sweden). The binding ability of a recombinant protein HER2 (Beijing ACROBiosystems, Cat: HE2-H5225) as an antigen to the conjugate BY12.7 is determined by surface plasmon resonance at 25° C. using HBS-EP (10 mM HEPES, pH 7.4, 150 mM NaCl, 3 mM EDTA, and 0.005% surfactant P20). Briefly, an anti-IgG antibody (GE Healthcare Life Sciences, cat: BR-1008-39) is immobilized directly onto a CM5 chip using a standard amine coupling kit, according to the manufacturer's instructions. The purified conjugate BY12.7 or the control antibody trastuzumab (purchased from Roche) is diluted in HEPES-buffered saline, specifically binds to chip-conjugated sheep anti-human IgG, and is injected at a flow rate of 5 l/min on a reaction matrix. A rate constant is obtained by kinetic binding measurements at different antigen concentrations within a range of 1.25 nM to 1000 nM. Based on kinetic information provided by the binding process and dissociation process in a sensing diagram, Biacore can detect a binding rate ka(1/ms), dissociation rate kd(1/s) and affinity KD(M) of a ligand to an analyte. Data analysis is performed using BIA evaluation software (BIAevaluation 4.1 software, from GE Healthcare Biosciences AB, Sweden) to obtain affinity data in Table 16.

TABLE 16
Binding of a conjugate to a second target

| Names of proteins | Target | ka (1/Ms) | kd (1/s) | KD (M) |
|---|---|---|---|---|
| Control antibody | HER2-A | 2.35E+4 | 3.56E−5 | 1.56E−9 |
| trastuzumab | | | | |
| Conjugate BY12.7 | | 6.38E+4 | 7.52E−5 | 1.17E−9 |

As can be seen from Table 16, the binding ability of the conjugate BY12.7 to the second target, HER2, is comparable to that of trastuzumab.

### Example 5. Construction and Functional Study of an IgG1 Fc-CD80 Fusion Protein Conjugate Having an Anti-GPC-3 Antibody Fab

### Example 5.1 Construction, Expression and Purification of an IgG1 Fc-CD80 Fusion Protein Conjugate Having an Anti-GPC-3 Antibody Fab

First, an anti-GPC-3 monoclonal antibody trastuzumab is prepared as a control.

According to an amino acid sequence of an anti-GPC-3 monoclonal antibody codrituzumab numbered 9759 in the International Nonproprietary Name (INN) database, the following nucleotide sequences are optimized to be suitable for expression in Chinese hamster ovary cancer cells (CHO), and Shanghai Jierui Bioengineering Co., Ltd. is entrusted to synthesize a nucleotide sequence. The nucleotide sequence is expressed to produce the antibody BY20.2 (i.e., codrituzumab).

Light-chain (BY20.2L) amino acid sequence of the ani-GPC-3 antibody BY20.2:
(SEQ ID NO: 115)
CTCGAGGCCACCATGGAGACCGACACACTCCTCCTGTGGGTGCTGCTGCTGTGGGTGCCTGGCTC
CACTGGCGACGTGGTGATGACACAGTCTCCACTGAGCCTCCCTGTGACACCAGGCGAGCCCGCC
TCGATCTCTTGCCGGTCCTCTCAGAGCCTCGTGCACTCTAACAGAAACACATACCTGCACTGGTAC
TTACAGAAGCCCGGCCAGTCCCCTCAGCTGCTGATCTACAAGGTGTCCAACAGATTCAGCGGCGT
GCCCGATCGTTTCAGCGGCAGCGGCTCCGGCACAGACTTCACACTGAAAATTTCACGTGTGGAG
GCTGAGGACGTGGGCGTGTACTACTGCTCTCAGAACACCCACGTGCCACCTACATTCGGCCAGGG
CACAAAGCTCGAAATCAAGAGAACCGTGGCCGCCCCATCCGTGTTCATCTTCCCACCATCCGACG
AGCAGCTCAAGTCTGGCACCGCGTCAGTGGTGTGCCTGCTGAACAACTTCTACCCTAGGGAGGCT
AAGGTGCAGTGGAAGGTGGACAACGCTTTGCAGTCTGGCAACTCCCAGGAGTCTGTGACCGAGC
AGGACTCTAAGGACTCCACATACAGCCTGTCCTCTACACTCACCCTGTCCAAGGCCGACTACGAG
AAGCACAAGGTGTACGCTTGCGAGGTGACCCACCAGGGCCTGTCTTCTCCAGTGACAAAGTCTT
TCAACCGGGGCGAGTGCTAAGAATTC
Light-chain (BY20.2L) amino acid sequence of an anti-GPC-3 antibody BY20.2:
(SEQ ID NO: 116)
METDTLLLWLLLWVPGSTGDVVMTQSPLSLPVTPGEPASISCRSSQSLVHSNRNTYLHWYLQKPGQS
PQLLIYKVSNRFSGVPDRFSGSGSGTDFTLKISRVEAEDVGVYYCSQNTHVPPTFGQGTKLEIKRTVAA
PSVFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Heavy-chain (BY20.2H) nucleotide sequence of the ani-GPC-3 antibody BY20.2:
(SEQ ID NO: 117)
TCTAGAGCCACCATGGAGACCGACACCCTGCTGCTGTGGGTGCTGCTCCTGTGGGTGCCTGGCTC
CACAGGCCAGGTGCAGCTCGTGCAGTCCGGCGCCGAGGTGAAGAAGCCCGGCGCTTCTGTGAAG
GTGTCTTGCAAGGCCTCAGGCTACACATTCACCGACTACGAGATGCACTGGGTGAGGCAGGCTCC
TGGCCAGGGCCTGGAGTGGATGGGCGCTCTCGACCCAAAGACCGGCGACACCGCTTACTCTCAG
AAGTTCAAGGGCCGGGTGACACTGACCGCCGACAAGAGCACATCTACCGCTTACATGGAGCTATC
ATCTCTGACCTCCGAGGACACCGCCGTGTACTACTGCACCAGATTCTACTCTTACACATACTGGGG
CCAGGGCACACTCGTGACAGTGTCTAGCGCCTCGACAAAGGGCCCATCTGTGTTCCCACTCGCTC
CATCTTCTAAGTCCACATCTGGCGGCACCGCCGCTCTCGGCTGCCTCGTGAAGGACTACTTCCCCG
AGCCTGTGACAGTGTCTTGGAACAGCGGCGCCCTCACATCCGGCGTGCACACCTTCCCCGCTGTG
TTGCAGTCTAGCGGCCTGTACTCCCTGAGCAGCGTGGTGACAGTGCCATCCTCTTCTCTGGGCAC
CCAGACATACATCTGCAACGTGAACCACAAGCCTAGCAACACAAAGGTGGACAAGAAGGTGGAG
CCTAAGTCTTGCGACAAGACCCACACATGCCCACCTTGCCCCGCCCCTGAGCTGCTGGGCGGCCC
ATCTGTGTTCCTGTTCCCTCCAAAGCCAAAGGACACCCTCATGATCAGCAGAACCCCAGAGGTGA
CATGCGTGGTGGTGGACGTGTCTCACGAGGACCCCGAGGTGAAGTTCAACTGGTACGTGGACGG
CGTGGAGGTGCACAACGCTAAGACCAAGCCACGGGAGGAGCAGTACAACTCTACCTACAGAGTG
GTGTCCGTGCTCACAGTGCTGCACCAGGACTGGCTGAACGGCAAGGAGTACAAGTGCAAGGTGT
CCAACAAGGCCCTGCCCGCTCCTATCGAAAAAACAATCAGCAAGGCTAAGGGCCAGCCACGGGA
GCCTCAGGTGTACACACTGCCACCTTCCAGGGACGAGCTGACCAAGAACCAGGTGTCCCTCACA
TGCCTCGTGAAGGGCTTCTACCCTAGCGACATCGCCGTGGAGTGGGAGTCTAACGGCCAGCCAG
AGAACAACTACAAGACCACCCCTCCAGTGCTCGACTCCGACGGCTCTTTCTTCCTGTACTCTAAG
CTGACCGTGGACAAGTCACGATGGCAGCAGGGCAACGTGTTCAGCTGCTCCGTGATGCACGAGG
CTCTCCACAACCACTACACCCAGAAGTCCCTGAGCCTGTCTCCTGGCAAGTAAGTCGAC
Heavy-chain (BY20.2H) amino acid sequence of an anti-GPC-3 antibody BY20.2:
(SEQ ID NO: 118)
METDTLLLWVLLLWVPGSTGQVQLVQSGAEVKKPGASVKVSCKASGYTFTDYEMHWVRQAPGQG
LEWMGALDPKTGDTAYSQKFKGRVTLTADKSTSTAYMELSSLTSEDTAVYYCTRFYSYTYWGQGTEV
TVSSASTKGPSVFPLAPSSKSTSGGTAALGCEVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGK
The underlined part ″METDTLLLWVLLLWVVPGG″ is a signal peptide sequence.

Shanghai Jierii Bioengineering Co., Ltd. synthesizes light-chain and heavy-chain encoding nucleotide sequences of the above antibody BY20.2 (codrituzumab). The light chain and the heavy chain in the encoding nucleotide sequence of the conjugate BY20.2 are subjected to XhoI-EcoRI double enzyme digestion and XhoI-SalI double enzyme digestion respectively and then linked to a glutamine synthase high-performance expression vector with a double expression box (Patent grant number: CN104195173B, obtained from Beijing Biyang Biotechnology Co., Ltd.). After a correct sequencing verification, an expression vector of the antibody BY20.2 (codrituzumab) is obtained for expression.

Next, an IgG1 Fc-CD80 fusion protein conjugate with an anti-GPC-3 antibody Fab is prepared.

The preparation method is similar to Example 4.1 above, except that the antibodies used are different. The resulting conjugate after expression is denoted herein as a conjugate BY20.3 (codrituzumab-CD80).

Heavy-chain nucleotide sequence of the conjugate BY20.3
(SEQ ID NO: 119)
TCTAGAGCCACCATGGAGACCGACACACTCCTCCTGTGGGTGCTGCTGCTGTGGGTGCCTGGCTC
CACTGGCCAGGTGCAGCTCGTGCAGTCCGGCGCCGAGGTGAAGAAGCCCGGCGCTTCTGTGAAG
GTGTCTTGCAAGGCCTCAGGCTACACATTCACCGACTACGAGATGCACTGGGTGAGGCAGGCTCC
TGGCCAGGGCCTGGAGTGGATGGGCGCTCTCGACCCAAAGACCGGCGACACCGCTTACTCTCAG
AAGTTCAAGGGCCGGGTGACACTGACCGCCGACAAGAGCACATCTACCGCTTACATGGAGCTATC
ATCTCTGACCTCCGAGGACACCGCCGTGTACTACTGCACCAGATTCTACTCTTACACATACTGGGG
CCAGGGCACACTCGTGACAGTGTCTAGCGCCTCGACAAAGGGCCCATCTGTGTTCCCACTCGCTC
CATCTTCTAAGTCCACATCTGGCGGCACCGCCGCTCTCGGCTGCCTCGTGAAGGACTACTTCCCCG
AGCCTGTGACAGTGTCTTGGAACAGCGGCGCCCTCACATCCGGCGTGCACACCTTCCCCGCTGTG
TTGCAGTCTAGCGGCCTGTACTCCCTGAGCAGCGTGGTGACAGTGCCATCCTCTTCTCTGGGCAC
CCAGACATACATCTGCAACGTGAACCACAAGCCTAGCAACACAAAGGTGGACAAGAAGGTGGAG
CCTAAGTCTTGCGACAAGACCCACACATGCCCACCTTGCCCCGCCCCTGAGCTGCTGGGCGGCCC
ATCTGTGTTCCTGTTCCCTCCAAAGCCAAAGGACACCCTCATGATCAGCAGAACCCCAGAGGTGA
CATGCGTGGTGGTGGACGTGTCTCACGAGGACCCCGAGGTGAAGTTCAACTGGTACGTGGACGG
CGTGGAGGTGCACAACGCTAAGACCAAGCCACGGGAGGAGCAGTACAACTCTACCTACAGAGTG
GTGTCCGTGCTCACAGTGCTGCACCAGGACTGGCTGAACGGCAAGGAGTACAAGTGCAAGGTGT
CCAACAAGGCCCTGCCCGCTCCTATCGAAAAAACAATCAGCAAGGCTAAGGGCCAGCCACGGGA
GCCTCAGGTGTACACACTGCCACCTTCCAGGGACGAGCTGACCAAGAACCAGGTGTCCCTCACA
TGCCTCGTGAAGGGCTTCTACCCTAGCGACATCGCCGTGGAGTGGGAGTCTAACGGCCAGCCAG
AGAACAACTACAAGACCACCCCTCCAGTGCTCGACTCCGACGGCTCTTTCTTCCTGTACTCTAAG
CTGACCGTGGACAAGTCAAGGGAACGATGGCAGCAGGGCAACGTGTTCAGCTGCTCCGTGATGC
ACGAGGCTCTCCACAACCACTACACCCAGAAGTCCCTGAGCCTGTCTCCTGGCAAGGGCGGCGG
AGGATCTGGCGGCGGAGGCAGTGGAGGCGGCGGAAGTGTGATCCATGTAACAAAAGAAGTGAA
GGAAGTGGCTACACTCTCTTGCGGCCACAACGTGTCCGTGGAGGAACTAGCTCAGACCCGGATCT
ATTGGCAGAAAGAAAAGAAGATGGTGCTGACCATGATGTCCGGCGACATGAACATTTGGCCAGA
GTACAAGAACCGCACAATTTTCGACATTACAAACAACCTCTCTATTGTGATTCTGGCTCTCAGGCC
TAGCGACGAGGGCACATACGAGTGCGTGGTGCTCAAGTACGAGAAGGACGCTTTCAAGCGGGAG
CACCTCGCTGAGGTGACCCTGTCCGTGAAGGCCGACTTCCCTACTCCATCTATCTCCGACTTCGAG
ATCCCTACCTCTAACATCAGGCGGATCATCTGCAGCACCTCCGGCGGCTTCCCAGAGCCTCACCTC
TCTTGGCTGGAGAACGGCGAGGAGCTGAACGCTATCAACACCACAGTGTCTCAGGACCCTGAGA
CCGAGCTGTACGCCGTGTCCTCTAAGCTCGACTTCAACATGACCACAAACCACTCCTTCATGTGC
CTGATCAAGTACGGCCACCTGAGAGTGAACCAGACATTCAACTGGAACACAACCAAGCAGGAGC
ACTTCCCATAAGTCGAC
Heavy-chain amino acid sequence of a conjugate BY20.3:
(SEQ ID NO: 120)
METDTLLLWVLLLWVPGSTGQVQLVQSGAEVKKPGASVKVSCKASGYTFTDYEMHWVRQAPGQG
LEWMGALDPKTGDTAYSQKFKGRVTLTADKSTSTAYMELSSLTSEDTAVYYCTRFYSYTYWGQGTLV
TVSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGKGGGG
SGGGGSGGGGSVIHVTKEVKEVATLSCGHNVSVEELAQTRIYWQKEKKMVLTMMSGDMNIWPEYK
NRTIFDITNNLSIVILALRPSDEGTYECVVLKYEKDAFKREHLAEVTLSVKADFPTPSISDFEIPTSNIRRI
ICSTSGGFPEPHLSWLENGEELNAINTTVSQDPETELYAVSSKLDFNMTTNHSFMCLIKYGHLRVNQTF
NWNTTKQEHFP
The amino acid sequence ″METDTLLLWVLLLWVPGSTG″ is a signal peptide.

Shanghai Jierui Bioengineering Co., Ltd. synthesizes a heavy-chain encoding nucleotide sequence of the above conjugate BY20.3. Similar to Example 4.1 above, the heavy-chain encoding nucleotide sequence of the conjugate BY20.3 is subjected to XbaI-SalI double enzyme digestion, and then linked to an expression vector to which the light-chain (BY20.2L) nucleotide sequence of the anti-GPC-3 antibody BY20.2 has been linked. Through the expression after a correct sequencing verification, the conjugate BY20.3 (codrituzumab) is obtained.

Similar to Example 3.1 above, the expression and purification of the conjugate BY20.3 and the antibody BY20.2 are performed, and the molecular weights are measured. The results are shown in Table 17 below, where theoretical and actual measured values of molecular weight are given. Due to the glycosylation of proteins in a eukaryotic expression system, the actual measured molecular weight is higher than the theoretical predicted value.

TABLE 17
Molecular weights of purified proteins

| | | Theoretical | Actual |
|---|---|---|---|
| | | predicted | measured |
| | | molecular | molecular |
| Names of | | weight | weight |
| proteins | Subunit | (kDa) | (kDa) |
| Conjugate | Light chain | 24.0 | 25 |
| BY20.3 | Heavy chain | 73.3 | 95.0 |
| Control antibody | Light chain | 24.0 | 25 |
| BY20.2 | Heavy chain | 48.8 | 50.0 |

### Example 5.2 Using Biacore T100 to Determine the Effect of the Conjugate BY20.3 on the Binding Ability to a Second Target

Similar to the above Example 4.2, except that the antigen used is a GPC-3 protein (a product from Beijing Yigiao Shenzhou Biotechnology Co., Ltd., Cat: 10088-H08H) and the control antibody used is BY20.2, affinity data in Table 18 is obtained.

TABLE 18
Binding of a conjugate to a second target

| Names of proteins | Target | ka (1/Ms) | kd (1/s) | KD (M) |
|---|---|---|---|---|
| Control antibody BY20.2 | GPC-3 | 3.64E+4 | 7.57E−5 | 2.08E−9 |
| Conjugate BY20.3 | | 3.72E+4 | 5.54E−5 | 1.49E−9 |

As can be seen from Table 18, the binding ability of the conjugate BY20.3 to the second target, GPC-3, is comparable to that of codrituzumab.

### Example 5.3 In Vivo Biological Activity Study of Conjugate BY20.3 that can Bind to CD80 Ligand and GPC-3

This embodiment investigates a tumor suppression effect and safety of the conjugate BY20.3, and compares anti-tumor activities of the conjugate BY20.3 and the antibody BY20.2 (codrituzumab).

Test Method:

Experiments are performed similarly to Example 3.3 above. The experimental groups are as follows: a solvent (PBS) group, a conjugate BY20.3 (13 mg/kg, ip, q3d×6) group, and an antibody BY20.2 (10 mg/kg, ip, q3d×6) group.

Results:
The effects of administration on the safety of mice:

during the treatment period, the mice in each group are ingested with food and water normally, and no abnormal behaviors appears. The mice generally exhibit good conditions; and no mice die.
In vivo efficacy of the conjugate BY20.3 on a human liver cancer model HUH7

The tumor volumes in the solvent group, the antibody BY20.2 group and the conjugate BY20.3 group are 1779±275 mm³, 1492±201 mm³ and 896±157 mm³, respectively. The tumor growth inhibition rates of the antibody BY20.2 group and the conjugate BY20.3 group are 10% and 55%, respectively, and the tumor volume of the conjugate BY20.3 group is significantly lower than that of the solvent group and the antibody BY20.2 group (p<0.05). It is indicated that the tumor treatment effect of the conjugate BY20.3 is better than that of the antibody BY20.2.

### Example 6. Construction and Functional Study of an IgG1 Fc-CD80 Fusion Protein Conjugate Having an Anti-Trop-2 Antibody Fab

### Example 6.1 Construction, Expression and Purification of an IgG1 Fc-CD80 Fusion Protein Conjugate Having an Anti-Trop-2 Antibody Fab

First, an anti-trop-2 monoclonal antibody sacituzumab is prepared as a control.

According to an amino acid sequence of an anti-trop-2 monoclonal antibody sacituzumab numbered 10418 in the International Nonproprietary Name (INN) database, the following nucleotide sequence is optimized to be suitable for expression in Chinese hamster ovary cancer cells (CHO), and Shanghai Jierui Bioengineering Co., Ltd. is entrusted to synthesize a nucleotide sequence. The nucleotide sequence is expressed to produce the antibody BY43 (i.e., sacituzumab).

Light-chain (BY43L) amino acid sequence of the ani-trop-2 antibody BY43:
(SEQ ID NO: 121)
CTCGAGGCCACCATGGAGACCGATACTCTGCTGCTCTGGGTGCTGCTGCTGTGGGTCCCCGGCTC
CACCGGCGACATCCAGCTGACCCAGTCCCCTAGCTCCCTCAGCGCGTCAGTGGGCGACCGGGTG
AGCATCACATGCAAGGCTTCTCAGGACGTGTCTATCGCCGTGGCTTGGTATCAGCAGAAGCCAGG
CAAGGCCCCAAAGCTGCTGATCTACTCCGCCTCTTACAGATACACCGGCGTGCCCGACAGATTCA
GCGGCTCTGGCTCCGGCACCGACTTCACACTGACAATCTCTAGCTTGCAACCCGAGGACTTCGCC
GTGTACTACTGCCAGCAGCACTACATCACACCACTGACATTCGGCGCTGGCACAAAGGTGGAGAT
CAAGAGAACAGTGGCCGCTCCTAGCGTGTTCATCTTCCCACCATCCGACGAGCAGCTGAAGAGC
GGCACCGCTTCCGTGGTGTGCCTGCTGAACAACTTCTACCCTAGGGAGGCTAAGGTGCAGTGGA
AGGTGGACAACGCCCTACAATCTGGCAACTCTCAGGAGTCTGTGACAGAGCAGGACTCTAAGGA
CTCCACATACTCTCTGTCTTCTACCCTCACACTGTCCAAGGCCGACTACGAGAAGCACAAGGTGT
ACGCTTGCGAGGTGACCCACCAGGGCCTGTCCTCTCCTGTGACCAAGTCTTTCAACCGGGGCGA
GTGCTAAGAATTC
Light-chain (BY43L) amino acid sequence of an anti-trop-2 antibody BY43:
(SEQ ID NO: 122)
METDTLLLWVLLLWVPGSTGDIQLTQSPSSLSASVGDRVSITCKASQDVSIAVAWYQQKPGKAPKLLI
YSASYRYTGVPDRFSGSGSGTDFTLTISSLQPEDFAVYYCQQHYITPLTFGAGTKVEIKRTVAAPSVFIF
PPSDEQLKSGTASVVCLLNNFYPREAKVQWKVDNALQSGNSQESVTEQDSKDSTYSLSSTLTLSKAD
YEKHKVYACEVTHQGLS SPVTKSFNRGEC
Heavy-chain nucleotide sequence of the ani-trop-2 antibody BY43:
(SEQ ID NO: 123)
TCTAGAGCCACCATGGAAACCGACACACTGCTGCTGTGGGTCCTGCTGCTGTGGGTGCCAGGCTC
TACAGGCCAGGTGCAGCTGCAACAGTCCGGCAGCGAGCTGAAGAAGCCAGGCGCAAGCGTGAA
GGTGTCTTGCAAGGCATCTGGCTACACATTCACCAACTACGGCATGAACTGGGTGAAGCAGGCTC
CTGGCCAGGGCCTCAAGTGGATGGGCTGGATCAACACATACACAGGCGAGCCAACATACACCGA
CGACTTCAAGGGCAGATTCGCTTTCAGCCTCGACACATCCGTGAGCACCGCTTACCTCCAGATCA
GCTCTCTGAAGGCCGACGACACCGCCGTGTACTTCTGCGCGAGAGGCGGCTTCGGCAGCTCTTAC
TGGTACTTCGACGTGTGGGGCCAGGGCTCTCTCGTGACAGTGTCCAGCGCATCAACAAAGGGCC
CATCTGTGTTCCCACTCGCTCCATCTTCTAAGTCTACATCCGGCGGCACCGCCGCCCTCGGCTGCC
TCGTGAAGGACTACTTCCCTGAGCCTGTGACCGTGTCTTGGAACTCTGGCGCCCTCACATCCGGC
GTGCACACATTCCCCGCTGTGCTCCAGTCTAGCGGCCTGTACTCTCTGTCCTCTGTGGTGACAGTG
CCATCTAGCTCCCTCGGCACCCAGACCTACATCTGCAACGTGAACCACAAGCCTTCTAACACAAA
GGTGGACAAGAGAGTGGAGCCTAAGTCTTGCGACAAGACCCACACATGCCCACCTTGCCCCGCC
CCCGAGCTGCTGGGCGGCCCATCTGTGTTCCTGTTCCCTCCAAAGCCAAAGGACACACTCATGAT
CAGCAGAACCCCAGAGGTGACATGCGTGGTGGTGGACGTGTCTCACGAGGACCCCGAGGTGAA
GTTCAACTGGTACGTGGACGGCGTGGAGGTGCACAACGCTAAGACCAAGCCACGGGAGGAGCA
GTACAACTCTACCTACCGGGTGGTGAGCGTGCTGACAGTGCTGCACCAGGACTGGCTCAACGGC
AAGGAGTACAAGTGCAAGGTGTCTAACAAGGCCCTGCCCGCTCCTATCGAAAAGACCATCAGCA
AGGCTAAGGGCCAGCCTAGAGAGCCTCAGGTGTACACACTGCCACCTTCTCGGGAGGAGATGAC
AAAGAACCAGGTGTCCCTGACATGCCTCGTGAAGGGCTTCTACCCTAGCGACATCGCCGTGGAGT
GGGAGTCTAACGGCCAGCCCGAGAACAACTACAAGACCACCCCACCTGTGCTCGACTCCGACGG
CTCTTTCTTCCTGTACTCTAAGCTGACAGTGGACAAGTCCAGATGGCAGCAGGGCAACGTGTTCT
CTTGCTCCGTGATGCACGAGGCTCTCCACAACCACTACACCCAGAAGTCCCTGAGCCTGTCCCCT
GGCAAGGGATAAGTCGAC
Heavy-chain amino acid sequence of an anti-trop-2 BY43:
(SEQ ID NO: 124)
METDTLLLWVLLLWVPGSTGQVQLQQSGSELKKPGASVKVSCKASGYTFTNYGMNWVKQAPGQG
LKWMGWINTYTGEPTYTDDFKGRFAFSLDTSVSTAYLQISSLKADDTAVYFCARGGFGSSYWYFDV
WGQGSLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQKSLSL
SPGK
The underlined part ″METDTLLLWVLLLWVVPGG″ is a signal peptide sequence.

Shanghai Jierii Bioengineering Co., Ltd. synthesizes light-chain and heavy-chain encoding nucleotide sequences of the above antibody BY43 (sacituzumab). The light chain and the heavy chain in the encoding nucleotide sequence of BY43 are subjected to XhoI-EcoRI double enzyme digestion and XhoI-SalI double enzyme digestion respectively and then linked to a glutamine synthase high-performance expression vector with a double expression box (Patent grant number: CN104195173B, obtained from Beijing Biyang Biotechnology Co., Ltd.). After a correct sequencing verification, an expression vector of the antibody BY43 (sacituzumab) is obtained for expression.

Next, an IgG1 Fc-CD80 fusion protein conjugate having an anti-trop-2 antibody Fab is prepared.

The preparation method is similar to Example 4.1 above, except that the antibodies used are different. The resulting conjugate after expression is denoted herein as a conjugate BY43.2 (sacituzumab-CD80).

Heavy-chain nucleotide sequence of the conjugate BY43.2
(SEQ ID NO: 125)
TCTAGAGCCACCATGGAGACCGACACACTCCTCCTGTGGGTGCTGCTGCTGTGGGTGCCTGGCTC
CACTGGCCAGGTGCAGCTCGTGCAGTCCGGCGCCGAGGTGAAGAAGCCCGGCGCTTCTGTGAAG
GTGTCTTGCAAGGCCTCAGGCTACACATTCACCGACTACGAGATGCACTGGGTGAGGCAGGCTCC
TGGCCAGGGCCTGGAGTGGATGGGCGCTCTCGACCCAAAGACCGGCGACACCGCTTACTCTCAG
AAGTTCAAGGGCCGGGTGACACTGACCGCCGACAAGAGCACATCTACCGCTTACATGGAGCTATC
ATCTCTGACCTCCGAGGACACCGCCGTGTACTACTGCACCAGATTCTACTCTTACACATACTGGGG
CCAGGGCACACTCGTGACAGTGTCTAGCGCCTCGACAAAGGGCCCATCTGTGTTCCCACTCGCTC
CATCTTCTAAGTCCACATCTGGCGGCACCGCCGCTCTCGGCTGCCTCGTGAAGGACTACTTCCCCG
AGCCTGTGACAGTGTCTTGGAACAGCGGCGCCCTCACATCCGGCGTGCACACCTTCCCCGCTGTG
TTGCAGTCTAGCGGCCTGTACTCCCTGAGCAGCGTGGTGACAGTGCCATCCTCTTCTCTGGGCAC
CCAGACATACATCTGCAACGTGAACCACAAGCCTAGCAACACAAAGGTGGACAAGAAGGTGGAG
CCTAAGTCTTGCGACAAGACCCACACATGCCCACCTTGCCCCGCCCCTGAGCTGCTGGGCGGCCC
ATCTGTGTTCCTGTTCCCTCCAAAGCCAAAGGACACCCTCATGATCAGCAGAACCCCAGAGGTGA
CATGCGTGGTGGTGGACGTGTCTCACGAGGACCCCGAGGTGAAGTTCAACTGGTACGTGGACGG
CGTGGAGGTGCACAACGCTAAGACCAAGCCACGGGAGGAGCAGTACAACTCTACCTACAGAGTG
GTGTCCGTGCTCACAGTGCTGCACCAGGACTGGCTGAACGGCAAGGAGTACAAGTGCAAGGTGT
CCAACAAGGCCCTGCCCGCTCCTATCGAAAAAACAATCAGCAAGGCTAAGGGCCAGCCACGGGA
GCCTCAGGTGTACACACTGCCACCTTCCAGGGACGAGCTGACCAAGAACCAGGTGTCCCTCACA
TGCCTCGTGAAGGGCTTCTACCCTAGCGACATCGCCGTGGAGTGGGAGTCTAACGGCCAGCCAG
AGAACAACTACAAGACCACCCCTCCAGTGCTCGACTCCGACGGCTCTTTCTTCCTGTACTCTAAG
CTGACCGTGGACAAGTCAAGGGAACGATGGCAGCAGGGCAACGTGTTCAGCTGCTCCGTGATGC
ACGAGGCTCTCCACAACCACTACACCCAGAAGTCCCTGAGCCTGTCTCCTGGCAAGGGCGGCGG
AGGATCTGGCGGCGGAGGCAGTGGAGGCGGCGGAAGTGTGATCCATGTAACAAAAGAAGTGAA
GGAAGTGGCTACACTCTCTTGCGGCCACAACGTGTCCGTGGAGGAACTAGCTCAGACCCGGATCT
ATTGGCAGAAAGAAAAGAAGATGGTGCTGACCATGATGTCCGGCGACATGAACATTTGGCCAGA
GTACAAGAACCGCACAATTTTCGACATTACAAACAACCTCTCTATTGTGATTCTGGCTCTCAGGCC
TAGCGACGAGGGCACATACGAGTGCGTGGTGCTCAAGTACGAGAAGGACGCTTTCAAGCGGGAG
CACCTCGCTGAGGTGACCCTGTCCGTGAAGGCCGACTTCCCTACTCCATCTATCTCCGACTTCGAG
ATCCCTACCTCTAACATCAGGCGGATCATCTGCAGCACCTCCGGCGGCTTCCCAGAGCCTCACCTC
TCTTGGCTGGAGAACGGCGAGGAGCTGAACGCTATCAACACCACAGTGTCTCAGGACCCTGAGA
CCGAGCTGTACGCCGTGTCCTCTAAGCTCGACTTCAACATGACCACAAACCACTCCTTCATGTGC
CTGATCAAGTACGGCCACCTGAGAGTGAACCAGACATTCAACTGGAACACAACCAAGCAGGAGC
ACTTCCCATAAGTCGAC
Heavy-chain amino acid sequence of a conjugate BY43.2:
(SEQ ID NO: 126)
METDTLLLWVLLLWVPGSTGQVQLVQSGAEVKKPGASVKVSCKASGYTFTDYEMHWVRQAPGQG
LEWMGALDPKTGDTAYSQKFKGRVTLTADKSTSTAYMELSSLTSEDTAVYYCTRFYSYTYWGQGTEV
TVSSASTKGPSVFPLAPSSKSTSGGTAALGCEVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGKGGGG
SGGGGSGGGGSVIHVTKEVKEVATLSCGHNVSVEELAQTRIYWQKEKKMVLTMMSGDMNIWPEYK
NRTIFDITNNLSIVILALRPSDEGTYECVVLKYEKDAFKREHLAEVTLSVKADFPTPSISDFEIPTSNIRRI
ICSTSGGFPEPHLSWLENGEELNAINTTVSQDPETELYAVSSKLDFNMTTNHSFMCLIKYGHLRVNQTF
NWNTTKQEHFP
The amino acid sequence ″METDTLLLWVLLLWVPGSTG″ is a signal peptide.

Shanghai Jierui Bioengineering Co., Ltd. synthesizes a heavy-chain encoding nucleotide sequence of the above conjugate BY43.2. Similar to Example 4.1 above, the heavy-chain encoding nucleotide sequence of the conjugate BY43.2 is subjected to XbaI-SalI double enzyme digestion, and then linked to an expression vector to which the light-chain (BY43L) nucleotide sequence of the anti-trop-2 antibody BY43 has been linked. Through the expression after a correct sequencing verification, the conjugate BY43.2 (Sacituzumab-CD8) is obtained.

Similar to Example 3.1 above, the expression and purification of the conjugate BY43.2 and the antibody BY43 are performed, and the molecular weights are measured. The results are shown in Table 19 below, where theoretical and actual measured values of molecular weight are given. Due to the glycosylation of proteins in a eukaryotic expression system, the actual measured molecular weight is higher than the theoretical predicted value.

TABLE 19
Molecular weights of purified proteins

| | | Theoretical | Actual |
|---|---|---|---|
| | | predicted | measured |
| | | molecular | molecular |
| | | weight | weight |
| Names of proteins | Subunit | (kDa) | (kDa) |
| Conjugate BY43.2 | Light chain | 23.3 | 25 |
| | Heavy chain | 74.0 | 95.0 |
| Control antibody | Light chain | 23.3 | 25 |
| BY43 | Heavy chain | 49.4 | 50 |

### Example 6.2 Using Biacore T100 to Determine the Effect of the Conjugate BY43.2 on the Binding Ability to a Second Target

Similar to the implementation in the above Example 4.2, except that the antigen used is a trop-2 protein (a product from Beijing Yiqiao Shenzhou Biotechnology Co., Ltd., Cat: 10088-H08H) and the control antibody used is BY43, and affinity data in Table 20 is obtained.

TABLE 20
Binding of a conjugate to a second target

| Names of proteins | Target | ka (1/Ms) | kd (1/s) | KD (M) |
|---|---|---|---|---|
| Control antibody BY43 | trop-2 | 5.63E+4 | 4.82E−5 | 8.57E−10 |
| Conjugate BY43.2 | | 4.26E+4 | 6.89E−5 | 1.62E−9 |

As can be seen from Table 20, the binding ability of the conjugate BY43.2 to the second target, trop-2, is comparable to that of sacituzumab ((Note: it is well known in this art that the biacore affinity is not much different within 2 times).

### Example 7. Construction and Functional Study of an IgG4 Fc-CD80 Fusion Protein Conjugate Having a Receptor Extracellular Functional Domain

### Example 7.1 Construction, Expression and Purification of an IgG4 Fc-CD80 Fusion Protein Conjugate Having a VEGF Receptor Extracellular Functional Domain

The human hVEGFR extracellular domain (VEGFR1-D2/VEGFR2-D3) and the amino acid sequence of the Fc-CD80 fusion protein are optimized to be suitable for expression in Chinese hamster ovary cancer cells (CHO), and Shanghai Jierui Bioengineering Co., Ltd. is entrusted to synthesize the following polynucleotide sequence of SEQ ID NO: 127. The resulting protein after expression of the nucleotide sequence is denoted herein as a conjugate BY24.22 (VEGFR-Fc-CD80).

Nucleotide sequence of the conjugate BY24.22 (VEGFR-Fc-CD80):
(SEQ ID NO: 127)
CTCGAGGCCACCATGGAGACCGACACACTCCTCCTGTGGGTGCTGCTGCTGTGGGTGCCTGGCTC
CACTGGCGCTTCCGACACCGGCCGCCCTTTCGTGGAGATGTACTCCGAGATCCCTGAGATCATCC
ACATGACCGAGGGCCGCGAGCTGGTGATCCCTTGCCGCGTGACCTCCCCTAACATCACCGTGACC
CTGAAGAAGTTCCCTCTGGACACCCTGATCCCTGACGGCAAGCGCATCATCTGGGACTCCCGCAA
GGGCTTCATCATCTCCAACGCCACCTACAAGGAGATCGGCCTGCTGACCTGCGAGGCCACCGTGA
ACGGCCACCTGTACAAGACCAACTACCTGACCCACCGCCAGACCAACACCATCATCGACGTGGT
GCTGTCCCCTTCCCACGGCATCGAGCTGTCCGTGGGCGAGAAGCTGGTGCTGAACTGCACCGCCC
GCACCGAGCTGAACGTGGGCATCGACTTCAACTGGGAGTACCCTTCCTCCAAGCACCAGCACAA
GAAGCTGGTGAACCGCGACCTGAAGACCCAGTCCGGCTCCGAGATGAAGAAGTTCCTGTCCACC
CTGACCATCGACGGCGTGACCCGCTCCGACCAGGGCCTGTACACCTGCGCCGCCTCCTCCGGCCT
GATGACCAAGAAGAACTCCACCTTCGTGCGCGTGCACGAGGAGTCCAAGTACGGCCCTCCTTGC
CCTCCTTGCCCTGCCCCTGAGTTCCTGGGCGGCCCTTCCGTGTTCCTGTTCCCTCCTAAGCCTAAG
GACACCCTGATGATCTCCCGCACCCCTGAGGTGACCTGCGTGGTGGTGGACGTGTCCCAGGAGG
ACCCTGAGGTGCAGTTCAACTGGTACGTGGACGGCGTGGAGGTGCACAACGCCAAGACCAAGCC
TCGCGAGGAGCAGTTCAACTCCACCTACCGCGTGGTGTCCGTGCTGACCGTGCTGCACCAGGAC
TGGCTGAACGGCAAGGAGTACAAGTGCAAGGTGTCCAACAAGGGCCTGCCTTCCTCCATCGAGA
AGACCATCTCCAAGGCCAAGGGCCAGCCTCGCGAGCCTCAGGTGTACACCCTGCCTCCTTCCCAG
GAGGAGATGACCAAGAACCAGGTGTCCCTGACCTGCCTGGTGAAGGGCTTCTACCCTTCCGACAT
CGCCGTGGAGTGGGAGTCCAACGGCCAGCCTGAGAACAACTACAAGACCACCCCTCCTGTGCTG
GACTCCGACGGCTCCTTCTTCCTGTACTCCCGCCTGACCGTGGACAAGTCCCGCTGGCAGGAGGG
CAACGTGTTCTCCTGCTCCGTGATGCACGAGGCCCTGCACAACCACTACACCCAGAAGTCCCTGT
CCCTGTCCCTGGGCAAGGGAGGCGGAGGATCTGGCGGCGGAGGCAGTGGAGGCGGCGGAAGTG
TCATCCATGTGACCAAAGAGGTCAAAGAAGTCGCAACTCTGTCTTGCGGACACAACGTCTCCGTC
GAAGAACTCGCCCAGACTAGAATATATTGGCAGAAGGAAAAGAAGATGGTGCTCACCATGATGTC
CGGCGATATGAACATTTGGCCTGAGTACAAGAACCGGACAATCTTCGATATTACTAATAACCTGAG
TATCGTGATTCTCGCTCTGCGCCCTAGCGACGAGGGCACATACGAGTGTGTGGTGCTGAAGTACG
AGAAGGATGCATTCAAGCGGGAGCACCTCGCAGAGGTGACACTCTCCGTGAAGGCCGACTTCCC
AACCCCATCTATCTCCGACTTCGAGATCCCTACCTCTAACATCAGGCGGATCATCTGCAGCACCTC
CGGCGGCTTCCCAGAGCCTCACCTCTCTTGGCTGGAGAACGGCGAGGAGCTGAACGCTATCAAC
ACCACAGTGTCTCAGGACCCTGAGACCGAGCTGTACGCCGTGTCCTCTAAGCTCGACTTCAACAT
GACCACAAACCACTCCTTCATGTGCCTGATCAAGTACGGCCACCTGAGAGTGAACCAGACATTCA
ACTGGAACACAACCAAGCAGGAGCACTTCCCATAAGAATTC
Amino acid sequence of the fusion protein BY24.22 (VEGFR- Fc-CD80, IgG4):
(SEQ ID NO: 128)
METDTLLLWVLLLWVPGSTGASDTGRPFVEMYSEIPEIIHMTEGRELVIPCRVTSPNITVTLKKFPLDT
LIPDGKRIIWDSRKGFIISNATYKEIGLLTCEATVNGHLYKTNYLTHRQTNTIIDVVLSPSHGIELSVGEK
LVLNCTARTELNVGIDFNWEYPSSKHQHKKLVNRDLKTQSGSEMKKFLSTLTIDGVTRSDQGLYTCA
ASSGLMTKKNSTFVRVHEESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGKGGGGSGGGGSGGGGSVIHVTKE
VKEVATLSCGHNVSVEELAQTRIYWQKEKKMVLTMMSGDMNIWPEYKNRTIFDITNNLSIVILALRPS
DEGTYECVVLKYEKDAFKREHLAEVTLSVKADFPTPSISDFEIPTSNIRRIICSTSGGFPEPHLSWLENG
EELNAINTTVSQDPETEEYAVSSKLDFNMTTNHSFMCLIKYGHLRVNQTFNWNTTKQEHFP
The amino acid sequence ″METDTLLLWVLLLWVPGSTG″ is a signal peptide.

The encoding nucleotide sequence of the conjugate BY24.22(VEGFR-Fc-CD80) is subjected to XhoI-EcoRI double enzyme digestion and then linked to a glutamine synthase high-performance expression vector with a double expression box (Patent grant number: CN104195173B, obtained from Beijing Biyang Biotechnology Co., Ltd.). After a correct sequencing verification, the recombinant vector is used for the expression of the conjugate BY24.22(VEGFR-Fc-CD80, IgG4).

Similar to Example 1.1 above, the expression and purification of the conjugate BY24.22 are performed, and the molecular weights are measured. The results are shown in Table 21 below, where theoretical and actual measured values of molecular weight are given. Due to the glycosylation of proteins in a eukaryotic expression system, the actual measured molecular weight is higher than the theoretical predicted value.

TABLE 21
Molecular weights of purified proteins

| | | | Theoretical | Actual |
|---|---|---|---|---|
| | | | predicted | measured |
| | | | molecular | molecular |
| | | | weight | weight |
| Names of proteins | Structural formula | Subunit | (kDa) | (kDa) |
| Conj ugate BY24.22 | VEGFR-Fc-CD80 | Any | 73.2 | 95.0 |
| | | subunit | | |
| Control protein 301-8 | VEGFR-Fc | Any | 48.5 | 62.0 |
| (aflibercept) | | subunit | | |

### Example 7.2 Using Biacore T100 to Determine the Effect of the Conjugate BY24.22 on the Binding Ability to a Second Target

Similar to the implementation in the above Example 4.2, except that the antigen used is VEGF165 (a product from Beijing Yigiao Shenzhou Biotechnology Co., Ltd., Cat: 11066-HNAH), and the control protein used is 301-8 (aflibercept), affinity data in Table 22 is obtained.

TABLE 22
Binding of a conjugate to a second target

| Names of proteins | Target | ka (1/Ms) | kd (1/s) | KD (M) |
|---|---|---|---|---|
| Conjugate BY24.22 | VEGF-A | 3.49E+6 | 2.99E-5 | 8.57E−12 |
| (VEGFR-FC-CD80) | | | | |
| Control protein 301-8 | | 5.89E+5 | 4.33E-5 | 7.35E−12 |
| (aflibercept) | | | | |

As can be seen from Table 22, the binding ability of the conjugate BY24.22 to the second target, VEGF-A, is comparable to that of 301-8 (aflibercept).

### Example 7.3 In Vivo Biological Activity Study of Conjugate BY24.22 that can Bind to CD80 Ligand and VEGF

This embodiment investigates a tumor suppression effect and safety of the conjugate BY24.22.

Test Method:

Experiments are performed similarly to Example 3.3 above. The experimental groups are as follows: a solvent (PBS) group, and a BY24.22 (VEGFR-Fc-CD80, 10 mg/kg, ip, q3d×6) group.

Results:
The effects of administration on the safety of mice:

during the treatment period, the mice in each group are ingested with food and water normally, and no abnormal behaviors appears. The mice generally exhibit good conditions; and no mice die.
In vivo efficacy of the conjugate BY24.22 on a human liver cancer model HUH7

The tumor volumes in the solvent group and the BY24.22 (VEGFR-FC-CD80) group are 1779±275 mm³ and 1063±187 mm³, respectively. The tumor growth inhibition rate of the conjugate BY24.22 group to the tumor is 42%, and the tumor volume is significantly lower than that of the solvent group (p<0.05). It is indicated that the conjugate BY24.22 has a significant treatment effect on tumors (see FIG. 9).

### Example 8. Construction and Functional Study of an IgG4 Fc-CD80 Fusion Protein Conjugate Having a Fab-Like Fragment

### Example 8.1 Construction, Expression and Purification of a Fab-Like Structure Having a TGFβII Receptor Extracellular Functional Domain and the IgG4 Fc-CD80 Fusion Protein Conjugate

According to the TGFβII receptor extracellular region sequence in Table 8 and the sequence of the CD80 extracellular domain in Table 1 as well as the IgG4 sequence in Table 2, the nucleotide sequence is optimized to be suitable for expression in Chinese hamster ovary cancer cells (CHO), and Shanghai Jierui Bioengineering Co., Ltd. is entrusted to synthesize the following polynucleotide sequence of SEQ ID NO: 129. The resulting conjugate BY41.6 is produced after expression of the nucleotide sequence.

Light-chain nucleotide sequence of conjugate BY41.6 (TGFβII receptor
extracellular domain + light-chain constant region, κ + CD80 extracellular
region):
(SEQ ID NO: 129)
CTCGAGGCCACCATGGAGACCGACACACTCCTCCTGTGGGTGCTGCTGCTGTGGGTGCCTGGCTC
CACTGGCGTGACTGACAACAACGGCGCTGTGAAGTTCCCACAGCTCTGCAAGTTCTGCGACGTG
AGATTCTCTACTTGCGACAACCAGAAGTCTTGCATGTCTAACTGCTCCATCACTAGCATTTGCGAG
AAGCCTCAGGAGGTGTGCGTGGCCGTGTGGCGCAAGAACGACGAGAACATTACACTGGAGACC
GTGTGCCACGACCCAAAGCTGCCATACCACGACTTCATCTTAGAGGACGCCGCATCTCCTAAGTG
CATTATGAAGGAGAAGAAGAAGCCCGGCGAGACTTTCTTCATGTGCTCATGTAGCTCTGACGAGT
GCAACGACAACATTATCTTCTCCGAGGAGTACAACACATCTAACCCTGGAGGCGGAGGATCTAGA
ACCGTGGCCGCCCCCAGCGTGTTCATCTTCCCTCCCAGCGACGAGCAGCTGAAGTCTGGCACCGC
CAGCGTGGTGTGCCTGCTGAACAACTTCTACCCCCGCGAGGCCAAGGTGCAGTGGAAGGTGGAC
AACGCCCTGCAGAGCGGCAACAGCCAGGAGAGCGTGACCGAGCAGGACTCCAAGGACAGCACC
TACAGCCTGAGCAGCACCCTGACCCTGAGCAAGGCCGACTACGAGAAGCACAAGGTGTACGCCT
GCGAGGTGACCCACCAGGGACTGTCTAGCCCCGTGACCAAGAGCTTCAACCGGGGCGAGTGCTA
AGAATTC
Light-chain amino acid sequence of conjugate BY41.6 (TGFβ II receptor
extracellular domain + light-chain constant region, κ + CD80 extracellular
region):
(SEQ ID NO: 130)
METDTLLLWVLLLWVPGSTGVTDNNGAVKFPQLCKFCDVRFSTCDNQKSCMSNCSITSICEKPQEVC
VAVWRKNDENITLETVCHDPKLPYHDFILEDAASPKCIMKEKKKPGETFFMCSCSSDECNDNIIFSEEY
NTSNPGGGGSRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
The amino acid sequence ″METDTLLLWVLLLWVPGSTG″ is a signal peptide.
Heavy-chain nucleotide sequence of conjugate BY41.6 (TGFβII receptor
extracellular domain + IgG heavy-chain constant region IgG4 + CD80
extracellular region):
(SEQ ID NO: 131)
TCTAGAGCCACCATGGAGACCGACACACTCCTCCTGTGGGTGCTGCTGCTGTGGGTGCCTGGCTC
CACTGGCGTGACTGACAACAACGGCGCTGTGAAGTTCCCACAGCTCTGCAAGTTCTGCGACGTG
AGATTCTCTACTTGCGACAACCAGAAGTCTTGCATGTCTAACTGCTCCATCACTAGCATTTGCGAG
AAGCCTCAGGAGGTGTGCGTGGCCGTGTGGCGCAAGAACGACGAGAACATTACACTGGAGACC
GTGTGCCACGACCCAAAGCTGCCATACCACGACTTCATCTTAGAGGACGCCGCATCTCCTAAGTG
CATTATGAAGGAGAAGAAGAAGCCCGGCGAGACTTTCTTCATGTGCTCATGTAGCTCTGACGAGT
GCAACGACAACATTATCTTCTCCGAGGAGTACAACACATCTAACCCTGGAGGCGGAGGATCTTCC
GCCTCCACCAAGGGCCCTTCCGTGTTCCCTCTGGCCCCTTGCTCCCGCTCCACCTCCGAGTCCAC
CGCCGCCCTGGGCTGCCTGGTGAAGGACTACTTCCCTGAGCCTGTGACCGTGTCCTGGAACTCCG
GCGCCCTGACCTCCGGCGTGCACACCTTCCCTGCCGTGCTGCAGTCCTCCGGCCTGTACTCCCTG
TCCTCCGTGGTGACCGTGCCTTCCTCCTCCCTGGGCACCAAGACCTACACCTGCAACGTGGACCA
CAAGCCTTCCAACACCAAGGTGGACAAGCGCGTGGAGTCCAAGTACGGCCCTCCTTGCCCTCCT
TGCCCTGCCCCTGAGTTCCTGGGCGGCCCTTCCGTGTTCCTGTTCCCTCCTAAGCCTAAGGACACC
CTGATGATCTCCCGCACCCCTGAGGTGACCTGCGTGGTGGTGGACGTGTCCCAGGAGGACCCTGA
GGTGCAGTTCAACTGGTACGTGGACGGCGTGGAGGTGCACAACGCCAAGACCAAGCCTCGCGA
GGAGCAGTTCAACTCCACCTACCGCGTGGTGTCCGTGCTGACCGTGCTGCACCAGGACTGGCTG
AACGGCAAGGAGTACAAGTGCAAGGTGTCCAACAAGGGCCTGCCTTCCTCCATCGAGAAGACCA
TCTCCAAGGCCAAGGGCCAGCCTCGCGAGCCTCAGGTGTACACCCTGCCTCCTTCCCAGGAGGA
GATGACCAAGAACCAGGTGTCCCTGACCTGCCTGGTGAAGGGCTTCTACCCTTCCGACATCGCCG
TGGAGTGGGAGTCCAACGGCCAGCCTGAGAACAACTACAAGACCACCCCTCCTGTGCTGGACTC
CGACGGCTCCTTCTTCCTGTACTCCCGCCTGACCGTGGACAAGTCCCGCTGGCAGGAGGGCAAC
GTGTTCTCCTGCTCCGTGATGCACGAGGCCCTGCACAACCACTACACCCAGAAGTCCCTGTCCCT
GTCCCTGGGCAAGGGAGGCGGAGGATCTGGCGGCGGAGGCAGTGGAGGCGGCGGAAGTGTGAT
CCATGTAACAAAAGAAGTGAAGGAAGTGGCTACACTCTCTTGCGGCCACAACGTGTCCGTGGAG
GAACTAGCTCAGACCCGGATCTATTGGCAGAAAGAAAAGAAGATGGTGCTGACCATGATGTCCGG
CGACATGAACATTTGGCCAGAGTACAAGAACCGCACAATTTTCGACATTACAAACAACCTCTCTA
TTGTGATTCTGGCTCTCAGGCCTAGCGACGAGGGCACATACGAGTGCGTGGTGCTCAAGTACGAG
AAGGACGCTTTCAAGCGGGAGCACCTCGCTGAGGTGACCCTGTCCGTGAAGGCCGACTTCCCTA
CTCCATCTATCTCCGACTTCGAGATCCCTACCTCTAACATCAGGCGGATCATCTGCAGCACCTCCG
GCGGCTTCCCAGAGCCTCACCTCTCTTGGCTGGAGAACGGCGAGGAGCTGAACGCTATCAACAC
CACAGTGTCTCAGGACCCTGAGACCGAGCTGTACGCCGTGTCCTCTAAGCTCGACTTCAACATGA
CCACAAACCACTCCTTCATGTGCCTGATCAAGTACGGCCACCTGAGAGTGAACCAGACATTCAAC
TGGAACACAACCAAGCAGGAGCACTTCCCATAAGTCGAC
Heavy-chain amino acid sequence of conjugate BY41.6 (TGFβII receptor
extracellular domain + IgG heavy-chain constant region, IgG4 + CD80
extracellular region):
(SEQ ID NO: 132)
METDTLLLWVLLLWVPGSTGVTDNNGAVKFPQLCKFCDVRFSTCDNQKSCMSNCSITSICEKPQEVC
VAVWRKNDENITLETVCHDPKLPYHDFILEDAASPKCIMKEKKKPGETFFMCSCSSDECNDNIIFSEEY
NTSNPGGGGSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLG
KGGGGSGGGGSGGGGSVIHVTKEVKEVATLSCGHNVSVEELAQTRIYWQKEKKMVLTMMSGDMNI
WPEYKNRTIFDITNNLSIVILALRPSDEGTYECVVLKYEKDAFKREHLAEVTLSVKADFPTPSISDFEIP
TSNIRRIICSTSGGFPEPHLSWLENGEELNAINTTVSQDPETELYAVSSKLDFNMTTNHSFMCLIKYGHL
RVNQTFNWNTTKQEHFP
The amino acid sequence ″METDTLLLWVLLLWVPGSTG″ is a signal peptide.

Shanghai Jierui Bioengineering Co., Ltd. synthesizes light-chain and heavy-chain encoding nucleotide sequences of the above conjugate BY41.6. The light chain and the heavy chain in the encoding nucleotide sequence of the conjugate BY41.6 are subjected to XhoI-EcoRI double enzyme digestion and XhoI-SalI double enzyme digestion respectively and then linked to a glutamine synthase high-performance expression vector with a double expression box (Patent grant number: CN104195173B, obtained from Beijing Biyang Biotechnology Co., Ltd.). After a correct sequencing verification, an expression vector of the conjugate BY41.6 is obtained for expression.

Similar to Example 4.1 above, the expression and purification of the conjugate BY41.6 are performed, and the molecular weights are measured. The results are shown in Table 23 below, where theoretical and actual measured values of molecular weight are given. Due to the glycosylation of proteins in a eukaryotic expression system, the actual measured molecular weight is higher than the theoretical predicted value.

TABLE 23
Molecular weights of purified proteins

| | | | Theoretical | Actual |
|---|---|---|---|---|
| | | | predicted | measured |
| | | | molecular | molecular |
| | Structural | | weight | weight |
| Names of proteins | formula | Subunit | (kDa) | (kDa) |
| Conjugate BY41.6 | IgG-like | Light chain | 25.7 | 26.0 |
| | TGF-βRII-CD80 | Heavy chain | 74.4 | 95.0 |
| | fusion protein | | | |

### Example 8.2 Using Biacore T100 to Determine the Effect of the Conjugate BY41.6 on the Binding Ability to a Second Target

Similar to the above Example 4.2, except that the antigen used is a TGF-β1 (a product from Beijing Yiqiao Shenzhou Biotechnology Co., Ltd., Cat: 10088-H08H), affinity data in Table 24 is obtained.

TABLE 24
Binding of a conjugate to a second target

| Names of protein | Target | ka (1/Ms) | kd (1/s) | KD (M) |
|---|---|---|---|---|
| Conjugate BY41.6 | TGF-β1 | 8.95E+4 | 6.65E−4 | 7.43E−9 |

The K_{D}(M) of the conjugate BY41.6 to TGF-β1 is 7.43 E-9, which belongs to high affinity binding.

Conclusion: the CD80 extracellular region is constructed at the C-terminal of the conjugate through an appropriate linking peptide, which does not affect the binding ability of a second functional molecule at the N-terminal of the conjugate to its corresponding receptor or ligand.

### Example 8.3 In Vivo Biological Activity Study of Conjugate BY41.6 that can Bind to CD80 Ligand and TGFβ1

This embodiment investigates a tumor suppression effect and safety of the conjugate BY41.6.

Test Method:

Similarly to the above example 3.3. The experimental groups are as follows: a solvent (PBS) group, and a conjugate BY41.6 (13 mg/kg, ip, q3d×6) group.

Results:
The effects of administration on the safety of mice:

during the treatment period, the mice in each group are ingested with food and water normally, and no abnormal behaviors appears. The mice generally exhibit good conditions; and no mice die.
In vivo efficacy of the conjugate BY41.6 on a human liver cancer model HUH7

At the end of the experiment, the tumor volumes in the solvent group and the BY24.22 group are 1779±275 mm³ and 967±97 mm³, respectively. The tumor growth inhibition rate of the conjugate BY41.6 group to the tumor is 45%, and the tumor volume is significantly lower than that of the solvent group (p<0.05). It is indicated that the conjugate BY41.6 has a significant treatment effect on tumors (see FIG. 9).

Conclusion:

The results of CD80 crystal structure analysis (PDB ID: 118L) show that CD80 participates in the binding of CD28, CTAL-4 and PDL-1 in a form of dimers on the cell surface, the IgV domain at the N-terminal is mainly involved in the binding effect, and IgC mainly maintains the stability of B7-1 and B7-2 (Truneh Al, et al. Mol Immunol, 1996, 33: 321-334; Kariv I, et al., J Immunol, 1996, 157: 29-38; Morton P A, et al., J Immunol., 1996 156: 1047-1054). WO2017/181152 improves the binding ability of CD80 to CTLA-4, PD-L1 and CD28 by forming an immune fusion protein of a selected CD80 extracellular region IgV mutant with IgG1 Fc, enhances immune activation to achieve the purpose of having a good growth inhibitory effect on tumors, and has a better inhibitory effect than that of the PD-L1 antibody.

The present invention has found that when the CD80 extracellular domain is placed at the N-terminal and C-terminal of Fc, respectively, two fusion proteins of different conformations are produced. Based on this, a fusion protein with the CD80 extracellular domain placed at the C-terminal of the Fc domain is conceived, which may “break apart” the CD80 dimer and hinder the formation of the CD80 dimer, thereby exposing more CD80 extracellular domain and thus promoting the binding of CD80 to CD28, CTLA-4 and PD-L1. The experimental results of the present invention show that the CD80 extracellular domain is located at the C-terminal of the Fc domain, which helps to improve the binding ability to CD28, CTLA-4 and PD-L1, thereby enhancing the immunostimulatory function of CD80.

1-14. (canceled)

15. An Fc-CD80 fusion protein, comprising an immunoglobulin Fc domain and a CD80 extracellular domain, wherein the CD80 extracellular domain is located at a C-terminal of the Fe domain.

16. The Fc-CD80 fusion protein according to claim 15, wherein the CD80 extracellular domain is linkered to the Fc domain by a linking peptide.

17. The Fc-CD80 fusion protein according to claim 16, wherein the amino acid sequence of the linker peptide is selected from any one of SEQ ID NO: 11-36, or (G4S)n, where N is 1, 2, 4 and 5.

18. The Fc-CD80 fusion protein according to claim 15, wherein the immunoglobulin Fc domain is a human or mouse Fc domain.

19. The Fc-CD80 fusion protein according to claim 18, wherein the human Fc domain is a Fc domain of human IgG1, IgG2, IgG3 or IgG4.

20. The Fc-CD80 fusion protein according to claim 19, wherein the Fc domain is is a Fc domain of an amino acid sequence shown in SEQ ID NOs: 8, 9 or 10, or a Fc domain having at least 90%, 91%, 92%, 93%, 94%, 95%, 96%, 97%, 98%, 99% or more sequence identity with the amino acid sequence shown in SEQ ID NO: 8, 9 or 10.

21. The Fc-CD80 fusion protein according to claim 15, wherein the CD80 extracellular domain is human CD80 ECD.

22. The Fc-CD80 fusion protein according to claim 21, wherein the human CD80 ECD comprises human CD80 IgV or human CD80 IgVIgC.

23. The Fc-CD80 fusion protein according to claim 22, wherein the human CD80 ECD has an amino acid sequence shown in SEQ ID NO: 1 or 2 or has at least 90%, 91%, 92%, 93%, 94%, 95%, 96%, 97%, 98%, 99% or more of the amino acid sequence shown in SEQ ID NO: 1 or 2.

24. A conjugate, comprising a first component and a second component, the second component is linked to the N-terminal of the first component; the first component is the Fc-CD80 fusion protein according to claim 15, The second component comprises a second effector molecule comprising an antibody fragment, a cellular extracellular receptor or an extracellular domain of the cellular extracellular receptor, a polypeptide comprising an extracellular domain of the cellular extracellular receptor or receptor, or other proteins.

25. The conjugate according to claim 24, wherein the antibody fragment comprises Fab, Fab′, F(ab′)₂, Fv, or single-chain Fv.

26. The conjugate according to claim 24, wherein The antibody fragment comprises antibody fragment that specifically bind to tumor-specific antigen or tumor-associated antigen, antibody fragment that specifically bind to immune checkpoint molecule of immune cells, or antibody fragment that specifically bind to immune agonist molecule of immune cells;
the tumor-specific antigen or tumor-associated antigen is selected from: an epidermal growth factor receptor (EGFR1), HER2/neu, CD20, a vascular endothelial growth factor (VEGF), an insulin-like growth factor receptor (IGF-1R), a TRAIL receptor, an epithelial cell adhesion molecule, a carcinoembryonic antigen, a prostate specific membrane antigen (PSMA), Mucin-1, CD30, CD33, Trop-2, CD40, CD137, Ang2, cMet; PDGF, DLL-4; CD138, CD19, CD133; CD38, CD22, ErbB3, angiopoietin-2 (Ang-2), TWEAK, CLDN18.2, CD73, MSTN (Myostatin, growth differentiation factor 8), AXL (AXL receptor tyrosine kinase), TNFRSF12A (Tumor Necrosis Factor Receptor (TNFR) Superfamily, member 12A), PVRL4 (4 associated with poliovirus receptor), MUC5AC (mucin 5AC), ITGAV_ITGB3 (Integrin αV_β3), FOLR1 (Folate Receptor 1), GPNMB (Glycoprotein (transmembrane) nmb), SDC1 (Syndecan-1), ENG (Endoglin), CA9 (Carbonic Anhydrase IX), PTPRC (Protein Tyrosine Phosphatase Receptor Type C), a DNA/histone (H1) complex, CEACAM5 (Carcinoembryonic Antigen-related Cell Adhesion Molecule 5), SLC39A6 (Solute Carrier Family 39, member 6), TNFRSF10B (Tumor Necrosis Factor Receptor (TNFR) Superfamily, member 10B), SLC34A2 (Solute Carrier Family 34 Sodium Phosphate, member 2), KIRD2 subgroup (Killer Cell Immunoglobulin-like Receptor from KIRD2 Subgroup), TNFRSF10A (Tumor Necrosis Factor Receptor (TNFR) Superfamily, member 10A), ganglioside GD3, CCR4 (Chemokine (C-C motif) Receptor 4), KLRC1 (Killer Cell Lectin-like Receptor Superfamily C, member 1), AMHR2 (Anti-Mullerian Hormone Receptor type 2), PDGFRA (Platelet-derived Growth Factor Receptor a Subunit), CD248 (Endothelin, Tumor Endothelial Marker 1), EGFL7 (Epidermal Growth Factor (EGF)-like Repeat Superfamily, member 7), CD79B (Immunoglobulin-associated CD790), ALCAM (Activated Leukocyte Adhesion Molecule CD166), VIM (Vimentin), MAG (Myelin-Associated Glycoprotein), PRLR (Prolactin Receptor), DLL3 (S-like 3), CD200 (OX-2), LRRC15 (Leucine-rich Repeat-containing Protein 15), SLITRK6 (SLIT and NTRK-like Family, member 6), FZD10 (Frizzled Class Receptor 10), NOTCH2 (Notch Protein 2), NOTCH3 (Notch Protein 3), EPCAM (Epithelial Cell Adhesion Molecule, tumor-associated calcium signaling transducer 1), ITGAV (Integrin αV), ACVRL1 (Activin A Receptor Type II Like Protein 1), CSF1R (Colony-stimulating Factor 1 Receptor), ACVR2A (Activin A Receptor Type IIA), MUC1 sialylated 5/8 carbohydrate tumor-associated (CA242), ganglioside GD2, EPHA3 (Ephrin Receptor A3), GUCY2C (Guanylate Cyclase 2C), PTPRC (Protein Tyrosine Phosphatase Receptor Type C), and IL1RAP (Interleukin 1 Receptor Accessory Protein);
immune checkpoint molecule is selected from: PD1, PD-L1, CTLA-4, TIM-3, LAG-3, TIGIT, STING, VISTA, CD47, or Siglec-15 (S15);
immune agonist molecule is selected from: GITR, 4-1BBL, OX40, ICOS, TLR2 or CD27.

27. The conjugate according to claim 24, wherein a Fab-like fragment that is formed by respectively linking CH1 of an immunoglobulin heavy chain and CL of an immunoglobulin light chain to a C-terminal of the extracellular functional domain of the receptor is used as the second component of the conjugate.

28. The conjugate according to claim 24, wherein the cellular extracellular receptor comprises a vascular endothelial growth factor receptor (VEGFR), a transforming growth factor 11 receptor, CD95, a lymphotoxin R receptor, an interleukin-1 receptor accessory protein, 4-1BBL, Lag-3, activin A receptor type II-like 1 (ALK1), AITRL, an IL-15 receptor a (IL15RA), a frizzled class receptor 8 (FZD8), an activin receptor type IIB, an activin A receptor type IIA, GITR, OX40, CD24 or CD40.

29. The conjugate according to claim 24, wherein the other protein is cell factor, the cell factor is selected from: IL-1, IL-2, IL-3, IL-4, IL-5, IL-6, IL-7, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28A, IL-28B, IL-29, IL-31, IL-32 and IL-33; a hematopoietic factor such as a macrophage colony-stimulating factor (M-CSF), a granulocyte macrophage colony-stimulating factor (GM-CSF), a granulocyte colony-stimulating factor (G-CSF) and erythropoietin; a tumor necrosis factor (TNF) such as TNF-α and TGF-β; a lymphokine such as lymphotoxin; a modulator for a metabolic process (e.g., leptin); interferon (e.g., IFN-α, IFN-β and IFN-γ); and a chemokine, preferably IL-2, IL-7, IL-15 or IL-33.

30. The conjugate according to claim 24, wherein The HER2 antibody fragment is the antibody fragment of trastuzumab; GPC-3 antibody fragment is an antibody fragment of cobaltuzumab; Trop-2 antibody fragment is the antibody fragment of sasituzumab.

31. A pharmaceutical composition, comprising the Fc-CD80 fusion protein according to claim 15.

32. The pharmaceutical composition according to claim 31, wherein the pharmaceutical composition further comprises a second therapeutic agent.

33. A method for treating or preventing cancerous diseases in an individual, wherein the method comprises administering an effective amount of the Fc-CD80 fusion protein according to claim 15 to a subject in need thereof.

34. The method according to claim 33, wherein the cancerous diseases comprise melanoma, breast cancer, colon cancer, esophageal cancer, gastrointestinal stromal tumor, renal cancer, liver cancer, non-small cell lung cancer, ovarian cancer, pancreatic cancer, prostate cancer, head and neck cancer, gastric cancer or hematological malignancies.


SEQUENCE LISTING (211 sequences)

SEQ ID NO: 1 - protein, 106 aa, Artificial sequence
  synthesized
  misc_feature: CD80-IgV
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKA

SEQ ID NO: 2 - protein, 208 aa, Artificial sequence [sequence omitted]

SEQ ID NO: 3 - protein, 337 aa, Artificial sequence
  synthesized
  misc_feature: IgG1 heavy chain constant region sequence
TLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 333 aa, Artificial sequence
  synthesized
  misc_feature: IgG2 heavy chain constant region sequence
TTVTVSTASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 334 aa, Artificial sequence
  synthesized
  misc_feature: IgG4 heavy chain constant region sequence
TLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 113 aa, Artificial sequence
  synthesized
  misc_feature: chain constant region sequence
TKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 112 aa, Artificial sequence
  synthesized
  misc_feature: chain constant region sequence
TKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 227 aa, Artificial sequence
  synthesized
  misc_feature: IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 228 aa, Artificial sequence
  synthesized
  misc_feature: IgG2 Fc region
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 229 aa, Artificial sequence
  synthesized
  misc_feature: IgG4 Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 16 aa, Artificial sequence
  synthesized
  misc_feature: linkage peptide
AKTTPKLEEGEFSEAR

SEQ ID NO: 12 - protein, 17 aa, Artificial sequence
  synthesized
  misc_feature: linkage peptide
AKTTPKLEEGEFSEARV

SEQ ID NO: 13 - protein, 9 aa, Artificial sequence
  synthesized
  misc_feature: linkage peptide
AKTTPKLGG

SEQ ID NO: 14 - protein, 10 aa, Artificial sequence
  synthesized
  misc_feature: linkage peptide
SAKTTPKLGG

SEQ ID NO: 15 - protein, 6 aa, Artificial sequence
  synthesized
  misc_feature: linkage peptide
SAKTTP

SEQ ID NO: 16 - protein, 6 aa, Artificial sequence
  synthesized
  misc_feature: linkage peptide
RADAAP

SEQ ID NO: 17 - protein, 9 aa, Artificial sequence
  synthesized
  misc_feature: linkage peptide
RADAAPTVS

SEQ ID NO: 18 - protein, 12 aa, Artificial sequence
  synthesized
  misc_feature: linkage peptide
RADAAAAGGPGS

SEQ ID NO: 19 - protein, 7 aa, Artificial sequence
  synthesized
  misc_feature: linkage peptide
RADAAAA

SEQ ID NO: 20 - protein, 18 aa, Artificial sequence
  synthesized
  misc_feature: linkage peptide
SAKTTPKLEEGEFSEARV

SEQ ID NO: 21 - protein, 5 aa, Artificial sequence
  synthesized
  misc_feature: linkage peptide
ADAAP

SEQ ID NO: 22 - protein, 11 aa, Artificial sequence
  synthesized
  misc_feature: linkage peptide
DAAPTVSIFPP

SEQ ID NO: 23 - protein, 5 aa, Artificial sequence
  synthesized
  misc_feature: linkage peptide
TVAAP

SEQ ID NO: 24 - protein, 12 aa, Artificial sequence
  synthesized
  misc_feature: linkage peptide
TVAAPSVFIFPP

SEQ ID NO: 25 - protein, 6 aa, Artificial sequence
  synthesized
  misc_feature: linkage peptide
QPKAAP

SEQ ID NO: 26 - protein, 13 aa, Artificial sequence
  synthesized
  misc_feature: linkage peptide
QPKAAPSVTLFPP

SEQ ID NO: 27 - protein, 6 aa, Artificial sequence
  synthesized
  misc_feature: linkage peptide
AKTTPP

SEQ ID NO: 28 - protein, 13 aa, Artificial sequence
  synthesized
  misc_feature: linkage peptide
AKTTPPSVTPLAP

SEQ ID NO: 29 - protein, 6 aa, Artificial sequence
  synthesized
  misc_feature: linkage peptide
AKTTAP

SEQ ID NO: 30 - protein, 13 aa, Artificial sequence
  synthesized
  misc_feature: linkage peptide
AKTTAPSVYPLAP

SEQ ID NO: 31 - protein, 6 aa, Artificial sequence
  synthesized
  misc_feature: linkage peptide
ASTKGP

SEQ ID NO: 32 - protein, 13 aa, Artificial sequence
  synthesized
  misc_feature: linkage peptide
ASTKGPSVFPLAP

SEQ ID NO: 33 - protein, 15 aa, Artificial sequence
  synthesized
  misc_feature: linkage peptide
GGGGSGGGGSGGGGS

SEQ ID NO: 34 - protein, 15 aa, Artificial sequence
  synthesized
  misc_feature: linkage peptide
GENKVEYAPALMALS

SEQ ID NO: 35 - protein, 15 aa, Artificial sequence
  synthesized
  misc_feature: linkage peptide
GPAKELTPLKEAKVS

SEQ ID NO: 36 - protein, 15 aa, Artificial sequence
  synthesized
  misc_feature: linkage peptide
GHEAAAVMQVQYPAS

SEQ ID NO: 37 - protein, 121 aa, Artificial sequence
  synthesized
  misc_feature: VH
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTV

SEQ ID NO: 38 - protein, 101 aa, Artificial sequence
  synthesized
  misc_feature: Bevacizumab VL
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQG

SEQ ID NO: 39 - protein, 113 aa, Artificial sequence
  synthesized
  misc_feature: Trastuzumab VH
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQG

SEQ ID NO: 40 - protein, 101 aa, Artificial sequence
  synthesized
  misc_feature: Trastuzumab VL
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQG

SEQ ID NO: 41 - protein, 112 aa, Artificial sequence
  synthesized
  misc_feature: Cetuximab VH
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQG

SEQ ID NO: 42 - protein, 101 aa, Artificial sequence
  synthesized
  misc_feature: Cetuximab VL
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAG

SEQ ID NO: 43 - protein, 114 aa, Artificial sequence
  synthesized
  misc_feature: Rituximab VH
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAG

SEQ ID NO: 44 - protein, 100 aa, Artificial sequence
  synthesized
  misc_feature: Rituximab VL
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGG

SEQ ID NO: 45 - protein, 108 aa, Artificial sequence
  synthesized
  misc_feature: Cobaltuzumab VH
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQG

SEQ ID NO: 46 - protein, 106 aa, Artificial sequence
  synthesized
  misc_feature: Cobaltuzumab VL
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQG

SEQ ID NO: 47 - protein, 106 aa, Artificial sequence
  synthesized
  misc_feature: Dinutuximab VH
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQG

SEQ ID NO: 48 - protein, 107 aa, Artificial sequence
  synthesized
  misc_feature: Dinutuximab VL
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAG

SEQ ID NO: 49 - protein, 105 aa, Artificial sequence
  synthesized
  misc_feature: VH
QVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQG

SEQ ID NO: 50 - protein, 101 aa, Artificial sequence
  synthesized
  misc_feature: VL
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQG

SEQ ID NO: 51 - protein, 109 aa, Artificial sequence
  synthesized
  misc_feature: Ozogamicin VH
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQG

SEQ ID NO: 52 - protein, 105 aa, Artificial sequence
  synthesized
  misc_feature: Ozogamicin VL
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQG

SEQ ID NO: 53 - protein, 114 aa, Artificial sequence
  synthesized
  misc_feature: CergutuzumabVH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQG

SEQ ID NO: 54 - protein, 102 aa, Artificial sequence
  synthesized
  misc_feature: CergutuzumabVL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQG

SEQ ID NO: 55 - protein, 111 aa, Artificial sequence
  synthesized
  misc_feature: Teprotumumab VH
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRG

SEQ ID NO: 56 - protein, 102 aa, Artificial sequence
  synthesized
  misc_feature: Teprotumumab VL
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQG

SEQ ID NO: 57 - protein, 114 aa, Artificial sequence
  synthesized
  misc_feature: Sacituzumab VH
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQG

SEQ ID NO: 58 - protein, 101 aa, Artificial sequence
  synthesized
  misc_feature: Sacituzumab VL
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAG

SEQ ID NO: 59 - protein, 106 aa, Artificial sequence
  synthesized
  misc_feature: Nivolumab VH
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQG

SEQ ID NO: 60 - protein, 101 aa, Artificial sequence
  synthesized
  misc_feature: Nivolumab VL
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQG

SEQ ID NO: 61 - protein, 113 aa, Artificial sequence
  synthesized
  misc_feature: Pembrolizumab VH
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQG

SEQ ID NO: 62 - protein, 105 aa, Artificial sequence
  synthesized
  misc_feature: Pembrolizumab VL
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGG

SEQ ID NO: 63 - protein, 111 aa, Artificial sequence
  synthesized
  misc_feature: Atezolizumab VH
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQG

SEQ ID NO: 64 - protein, 101 aa, Artificial sequence
  synthesized
  misc_feature: Atezolizumab VL
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQG

SEQ ID NO: 65 - protein, 111 aa, Artificial sequence
  synthesized
  misc_feature: Ipilimumab VH
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQG

SEQ ID NO: 66 - protein, 102 aa, Artificial sequence
  synthesized
  misc_feature: Ipilimumab VL
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQG

SEQ ID NO: 67 - protein, 106 aa, Artificial sequence
  synthesized
  misc_feature: ObolimabVH
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQG

SEQ ID NO: 68 - protein, 98 aa, Artificial sequence
  synthesized
  misc_feature: ObolimabVL
DIQMTQSPSSLSASVGDRVTITCRASQSIRRYLNWYHQKPGKAPKLLIYGASTLQSGRFSGSGSGTDFTLTISSLQPEDFAVYYCQQSHSAPLTFGGG

SEQ ID NO: 69 - protein, 113 aa, Artificial sequence
  synthesized
  misc_feature: relatlimab VH
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQG

SEQ ID NO: 70 - protein, 101 aa, Artificial sequence
  synthesized
  misc_feature: relatlimab VL
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQG

SEQ ID NO: 71 - protein, 119 aa, Artificial sequence
  synthesized
  misc_feature: tiragolumab VH
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQG

SEQ ID NO: 72 - protein, 107 aa, Artificial sequence
  synthesized
  misc_feature: tiragolumab VL
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPG

SEQ ID NO: 73 - protein, 110 aa, Artificial sequence
  synthesized
  misc_feature: magrolimab VH
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQG

SEQ ID NO: 74 - protein, 106 aa, Artificial sequence
  synthesized
  misc_feature: magrolimab VL
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQG

SEQ ID NO: 75 - protein, 114 aa, Artificial sequence
  synthesized
  misc_feature: UrelumabVH
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRG

SEQ ID NO: 76 - protein, 103 aa, Artificial sequence
  synthesized
  misc_feature: UrelumabVL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGG

SEQ ID NO: 77 - protein, 111 aa, Artificial sequence
  synthesized
  misc_feature: ivuxolimab VH
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQG

SEQ ID NO: 78 - protein, 101 aa, Artificial sequence
  synthesized
  misc_feature: ivuxolimab VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGG

SEQ ID NO: 79 - protein, 110 aa, Artificial sequence
  synthesized
  misc_feature: VopratelimabVH
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQG

SEQ ID NO: 80 - protein, 105 aa, Artificial sequence
  synthesized
  misc_feature: VopratelimabVL
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPG

SEQ ID NO: 81 - protein, 112 aa, Artificial sequence
  synthesized
  misc_feature: VarlilumabVH
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQG

SEQ ID NO: 82 - protein, 101 aa, Artificial sequence
  synthesized
  misc_feature: VarlilumabVL
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQG

SEQ ID NO: 83 - protein, 111 aa, Artificial sequence
  synthesized
  misc_feature: TomaralimabVH
EVQLVQSGSELKKPGASVKLSCKASGFTFTTYGINWVRQAPGQGLEWIGWIYPRDGSTNFNENFKDRATITVDTSASTAYMELSSLRSEDTAVYFCARLTGGTFLDYWGQG

SEQ ID NO: 84 - protein, 105 aa, Artificial sequence
  synthesized
  misc_feature: TomaralimabVL
DIVLTQSPATLSLSPGERATLSCRASESVEYYGTSLMQWYQQKPGQPPKLLIFGASNVESGVPDRFSGSGSGTDFTLKISRVEAEDVGMYFCQQSRKLPWTFGGG

SEQ ID NO: 85 - protein, 136 aa, Artificial sequence
  synthesized
  misc_feature: Extracellular region of transforming growth factor beta  receptor
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 86 - protein, 205 aa, Artificial sequence
  synthesized
  misc_feature: VEGFR extracellular region
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 87 - protein, 148 aa, Artificial sequence
  synthesized
  misc_feature: CD95 extracellular region
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSC

SEQ ID NO: 88 - protein, 329 aa, Artificial sequence
  synthesized
  misc_feature: Extracellular region of interleukin-1 receptor helper protein
GAARSCRFRGRHYKREFRLEGEPVALRCPQVPYWLWASVSPRINLTWHKNDSARTVPGEEETRMWAQDGALWLLPALQEDSGTYVCTTRNASYCDKMSIEFRVFENTDAFLPFISYPQILTLSTSGVLVCPDLSEFTRDKTDVKIQWYRDSLLLDKDNEKFLSVRGTTHLLVHDVAQEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWG

SEQ ID NO: 89 - protein, 106 aa, Artificial sequence
  synthesized
  misc_feature: Extracellular region of activin receptor-like kinase 1(ALK1)
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHT

SEQ ID NO: 90 - protein, 427 aa, Artificial sequence
  synthesized
  misc_feature: LAG3 extracellular region
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGDDDDKGSGSGEPKSC

SEQ ID NO: 91 - protein, 164 aa, Artificial sequence
  synthesized
  misc_feature: AITRL extracellular region
VSRLEEEMRKLQATVQELQKRLDRLEETVQAKGGGGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKDNTYWGIILLANPQFIS

SEQ ID NO: 92 - protein, 70 aa, Artificial sequence
  synthesized
  misc_feature: IL-15RA extracellular region
ITCPPPMSVEHADIWVKSYSLYSRERYICNSGFKRKAGTSSLTECVLNKATNVAHWTTPSLKCIREPKSC

SEQ ID NO: 93 - protein, 134 aa, Artificial sequence
  synthesized
  misc_feature: FZD8 extracellular region
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPK

SEQ ID NO: 94 - protein, 653 aa, Artificial sequence
  synthesized
  misc_feature: IL1RAP extracellular region
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSG

SEQ ID NO: 95 - protein, 129 aa, Artificial sequence
  synthesized
  misc_feature: GITRL extracellular region
LQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 96 - protein, 30 aa, Artificial sequence
  synthesized
  misc_feature: CD24 extracellular region
SETTTGTSSNSSQSTSNSGLAPNPTNATTK

SEQ ID NO: 97 - protein, 133 aa, Artificial sequence [sequence omitted]

SEQ ID NO: 98 - protein, 155 aa, Artificial sequence
  synthesized
  misc_feature: IL-7
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 99 - protein, 160 aa, Artificial sequence
  synthesized
  misc_feature: IL-10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 100 - protein, 159 aa, Artificial sequence
  synthesized
  misc_feature: IL-33
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 101 - DNA, 1431 bp, Artificial sequence
  synthesized
  misc_feature: Nucleotide sequence of fusion protein BY24.30(Fc-hCD80)
ctcgaggccaccatggaaaccgacacactgctgctgtgggtcctgctgctgtgggtgccaggctctacaggcgagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggcaagggaggcggaggatctggcggcggaggcagtggaggcggcggaagtgtgatccatgtaacaaaagaagtgaaggaagtggctacactctcttgcggccacaacgtgtccgtggaggaactagctcagacccggatctattggcagaaagaaaagaagatggtgctgaccatgatgtccggcgacatgaacatttggccagagtacaagaaccgcacaattttcgacattacaaacaacctctctattgtgattctggctctcaggcctagcgacgagggcacatacgagtgcgtggtgctcaagtacgagaaggacgctttcaagcgggagcacctcgctgaggtgaccctgtccgtgaaggccgacttccctactccatctatctccgacttcgagatccctacctctaacatcaggcggatcatctgcagcacctccggcggcttcccagagcctcacctctcttggctggagaacggcgaggagctgaacgctatcaacaccacagtgtctcaggaccctgagaccgagctgtacgccgtgtcctctaagctcgacttcaacatgaccacaaaccactccttcatgtgcctgatcaagtacggccacctgagagtgaaccagacattcaactggaacacaaccaagcaggagcacttcccataagaattc

SEQ ID NO: 102 - protein, 470 aa, Artificial sequence
  synthesized
  misc_feature: Amino Acid Sequence of Fusion Protein BY24.30 (Fc-hCD80, IgG4)
METDTLLLWVLLLWVPGSTGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 103 - DNA, 1389 bp, Artificial sequence
  synthesized
  misc_feature: Nucleotide sequence of fusion protein BY24.23(hCD80-Fc, IgG4)
ctcgaggccaccatggagaccgacacactcctcctgtgggtgctgctgctgtgggtgcctggctccactggcgtcatccatgtgaccaaagaggtcaaagaagtcgcaactctgtcttgcggacacaacgtctccgtcgaagaactcgcccagactagaatatattggcagaaggaaaagaagatggtgctcaccatgatgtccggcgatatgaacatttggcctgagtacaagaaccggacaatcttcgatattactaataacctgagtatcgtgattctcgctctgcgccctagcgacgagggcacatacgagtgtgtggtgctgaagtacgagaaggatgcattcaagcgggagcacctcgcagaggtgacactctccgtgaaggccgacttcccaaccccatctatctccgacttcgagatccctacctctaacatcaggcggatcatctgcagcacctccggcggcttcccagagcctcacctctcttggctggagaacggcgaggagctgaacgctatcaacaccacagtgtctcaggaccctgagaccgagctgtacgccgtgtcctctaagctcgacttcaacatgaccacaaaccactccttcatgtgcctgatcaagtacggccacctgagagtgaaccagacattcaactggaacacaaccaagcaggagcacttcccagacgagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggcaagtaagaattc

SEQ ID NO: 104 - protein, 456 aa, Artificial sequence
  synthesized
  misc_feature: Amino acid sequence of fusion protein BY24.23(hCD80-Fc, IgG4)
METDTLLLWVLLLWVPGSTGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - DNA, 1452 bp, Artificial sequence
  synthesized
  misc_feature: Nucleotide sequence of fusion protein BY24.24(Fc-mCD80, IgG2a)
ctcgaggccaccatggagacagacacactcctcctgtgggtgctcctgctctgggtgccaggcagcacaggcgagcctaggggccctaccatcaagccttgccccccatgcaagtgccccgctccaaacctcctcggcggcccttctgtgttcatcttccctccaaagatcaaggacgtgctgatgatcagcctgtccccaatcgtgacatgcgtggtggtggacgtgtccgaggacgacccagacgtgcagatctcttggttcgtgaacaacgtggaggtgcacaccgctcagacccagacccaccgggaggactacaactctacactgagagtggtgtccgccctgcctatccagcaccaggactggatgtctggcaaggagttcaagtgcaaggtgaacaacaaggacctgcccgctcctatcgagagaaccatctctaagccaaagggctctgtgcgggctcctcaggtgtacgtgctgcctccacctgaggaggagatgacaaagaagcaggtgacactgacatgcatggtgaccgacttcatgccagaggacatctacgtggagtggaccaacaacggcaagaccgagctgaactacaagaacacagagccagtgctcgacagcgacggctcttacttcatgtactctaagctgagagtggagaagaagaactgggtggagcggaactcttactcttgcagcgtggtgcacgagggcctgcacaaccaccacaccacaaagtctttctcccggaccccaggcaagggaggcggaggatctggcggcggaggcagtggaggcggcggaagtgtggacgagcagctgtctaagtctgtgaaggacaaggtgctgctgccttgccggtacaactcccctcacgaggacgagtccgaggaccggatctactggcagaagcacgacaaggtggtgctcagcgtgatcgccggcaagctgaaggtgtggcccgagtacaagaaccggacactgtacgacaacaccacatacagcctgatcatcctgggcctcgtgctgagcgaccggggcacatactcttgcgtggtgcagaagaaggagcggggcacatacgaggtgaagcacctcgccctcgtgaagctgtctatcaaggccgacttctctacccctaacatcacagagtctggcaaccctagcgccgacacaaagcggatcacatgcttcgctagcggcggcttccctaagccacggttctcttggttggaaaacggccgggagctgccaggcatcaacaccaccatctctcaggacccagagtccgagctgtacacaatctctagccagctcgacttcaacaccacccggaaccacacaatcaagtgcctcatcaagtacggcgacgctcacgtgtctgaggacttcacatgggagaagccacctgaggaccctcccgactctaagaactaagaattc

SEQ ID NO: 106 - protein, 477 aa, Artificial sequence
  synthesized
  misc_feature: Amino acid sequence of fusion protein BY24.24(Fc-mCD80, IgG2a)
METDTLLLWVLLLWVPGSTGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSGGGGSVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 107 - DNA, 723 bp, Artificial sequence
  synthesized
  misc_feature: The light chain (BY24.26L) of nucleotide sequenceBY24.26 (bevacizumab -CD80)
ctcgaggccaccatggagacagatactctgctgctctgggtcctgctgctgtgggtgccaggcagcacaggcgacatccagatgacccagtccccttcctccctgtctgcctccgtgggcgacagggtgaccatcacctgttccgcatcccaggatatctccaactacctgaactggtatcagcagaagccaggaaaggcccccaaggtgctgatctactttacttcttccctgcattccggcgtgccctccaggttctctggctccgggtctggaaccgactttacactgacaatctcctctctgcagccagaagactttgccacctattactgccagcagtactccactgtcccctggaccttcgggcaaggaaccaaagtggaaatcaagaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaagaattc

SEQ ID NO: 108 - protein, 234 aa, Artificial sequence
  synthesized
  misc_feature: The light chain (BY24.26l) amino acid sequence of conjugate by24.26 (bevacizumab -CD80)
METDTLLLWVLLLWVPGSTGDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - DNA, 2103 bp, Artificial sequence
  synthesized
  misc_feature: Heavy chain nucleotide sequence of conjugate BY24.26 (bevacizumab -CD80)
tctagagccaccatggaaaccgacacactgctgctgtgggtcctgctgctgtgggtgccaggctctacaggcgaagtgcagctggtcgaatctggcggcggactggtgcaaccagggggcagcctgaggctgtcctgcgccgcttctggatacacctttaccaattacggcatgaattgggtgaggcaggctcctggcaagggcctggagtgggtgggatggatcaacacctacaccggcgagcccacctacgccgctgactttaagaggaggttcaccttctctctggacacctccaagtccaccgcttacctgcaaatgaactctctgagggctgaagacaccgccgtgtactactgcgccaagtacccccattactacgggtcctcccactggtacttcgacgtgtggggccaaggcactctggtcaccgtgagctccgccagcaccaagggccccagcgtcttcccactggctccttcctctaaaagcactagcggagggaccgcagccctgggctgtctggtgaaagactacttccccgagcccgtgaccgtctcctggaactctggagccctgacctccggggtgcacacctttcccgccgtgctgcagtcttctggactgtactccctgtcctccgtcgtgactgtgcccagctcctccctgggaactcagacatacatctgcaacgtgaaccacaagccttccaacacaaaggtggacaagaaagtcgagcccaagagctgtgataagacccatacatgtcccccatgccccgctccagaactgctgggcggaccttccgtgtttctgttcccacccaaaccaaaggacacactgatgatcagcagaacccctgaggtgacttgcgtggtcgtggacgtgagccatgaggaccccgaggtgaagttcaactggtatgtggatggcgtggaagtgcataatgccaagacaaaacctagggaagagcagtacaacagcacctacagggtggtgagcgtgctgaccgtgctgcaccaggattggctgaacggcaaggaatacaagtgcaaggtgtccaataaggctctgcctgcacctatcgagaagaccatcagcaaagccaagggccaacccagagagcctcaagtctacaccctgcccccaagcagggaggagatgaccaaaaatcaagtgagcctgacatgcctggtcaaaggcttctaccctagcgacatcgccgtggagtgggagagcaatggccagcctgagaacaactacaagaccactccccccgtcctggatagcgacggcagcttcttcctgtactccaaactgacagtcgataaaagcaggtggcagcaaggcaatgtctttagctgtagcgtgatgcacgaggccctgcataaccactacactcaaaagtccctgtccctgagccccggaaagggaggcggaggatctggcggcggaggcagtggaggcggcggaagtgtgatccatgtaacaaaagaagtgaaggaagtggctacactctcttgcggccacaacgtgtccgtggaggaactagctcagacccggatctattggcagaaagaaaagaagatggtgctgaccatgatgtccggcgacatgaacatttggccagagtacaagaaccgcacaattttcgacattacaaacaacctctctattgtgattctggctctcaggcctagcgacgagggcacatacgagtgcgtggtgctcaagtacgagaaggacgctttcaagcgggagcacctcgctgaggtgaccctgtccgtgaaggccgacttccctactccatctatctccgacttcgagatccctacctctaacatcaggcggatcatctgcagcacctccggcggcttcccagagcctcacctctcttggctggagaacggcgaggagctgaacgctatcaacaccacagtgtctcaggaccctgagaccgagctgtacgccgtgtcctctaagctcgacttcaacatgaccacaaaccactccttcatgtgcctgatcaagtacggccacctgagagtgaaccagacattcaactggaacacaaccaagcaggagcacttcccataagtcgac

SEQ ID NO: 110 - protein, 694 aa, Artificial sequence
  synthesized
  misc_feature: Heavy chain amino acid sequence of conjugate BY24.26 (bevacizumab -CD80)
METDTLLLWVLLLWVPGSTGEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 111 - DNA, 723 bp, Artificial sequence
  synthesized
  misc_feature: The light chain (BY12.7l) nucleotide sequence of conjugate by12.7
ctcgaggccaccatggagaccgatactctgctgctctgggtgctgctgctgtgggtccccggctccaccggcgacatccagatgacccaatccccctcctctctgtctgcctccgtgggagatagggtgaccatcacatgcagggcatctcaggacgtcaacaccgctgtcgcttggtaccagcaaaagcccggaaaggcccccaagctgctgatttacagcgcttcctttctgtattccggcgtgccctccaggttttctggaagcaggagcgggaccgattttaccctgacaatctccagcctgcaacccgaggacttcgccacctactattgccagcagcactacaccacaccccctacattcggccaaggcaccaaggtggagatcaagaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaagaattc

SEQ ID NO: 112 - protein, 234 aa, Artificial sequence
  synthesized
  misc_feature: Amino acid sequence of light chain (BY12.7L) of conjugate by12.7
METDTLLLWVLLLWVPGSTGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - DNA, 2094 bp, Artificial sequence
  synthesized
  misc_feature: Heavy chain nucleotide sequence of conjugate BY12.7
tctagagccaccatggaaaccgacacactgctgctgtgggtcctgctgctgtgggtgccaggctctacaggcgaggtccaactggtggagtccggagggggactggtgcaacctggcggcagcctgaggctgtcctgcgctgcctccggcttcaacatcaaggacacctacattcactgggtgaggcaagcacccggaaagggcctggagtgggtggccaggatctaccccaccaacggctacaccaggtacgccgactccgtgaagggcaggttcaccatctccgccgatacctccaagaacaccgcctacctgcaaatgaactccctgagggccgaagacacagccgtgtactactgctccaggtggggaggggacggcttctacgccatggactattggggccaaggcactctggtcaccgtgagctccgccagcaccaagggccccagcgtcttcccactggctccttcctctaaaagcactagcggagggaccgcagccctgggctgtctggtgaaagactacttccccgagcccgtgaccgtctcctggaactctggagccctgacctccggggtgcacacctttcccgccgtgctgcagtcttctggactgtactccctgtcctccgtcgtgactgtgcccagctcctccctgggaactcagacatacatctgcaacgtgaaccacaagccttccaacacaaaggtggacaagaaagtcgagcccaagagctgtgataagacccatacatgtcccccatgccccgctccagaactgctgggcggaccttccgtgtttctgttcccacccaaaccaaaggacacactgatgatcagcagaacccctgaggtgacttgcgtggtcgtggacgtgagccatgaggaccccgaggtgaagttcaactggtatgtggatggcgtggaagtgcataatgccaagacaaaacctagggaagagcagtacaacagcacctacagggtggtgagcgtgctgaccgtgctgcaccaggattggctgaacggcaaggaatacaagtgcaaggtgtccaataaggctctgcctgcacctatcgagaagaccatcagcaaagccaagggccaacccagagagcctcaagtctacaccctgcccccaagcagggaggagatgaccaaaaatcaagtgagcctgacatgcctggtcaaaggcttctaccctagcgacatcgccgtggagtgggagagcaatggccagcctgagaacaactacaagaccactccccccgtcctggatagcgacggcagcttcttcctgtactccaaactgacagtcgataaaagcaggtggcagcaaggcaatgtctttagctgtagcgtgatgcacgaggccctgcataaccactacactcaaaagtccctgtccctgagccccggaaagggaggcggaggatctggcggcggaggcagtggaggcggcggaagtgtgatccatgtaacaaaagaagtgaaggaagtggctacactctcttgcggccacaacgtgtccgtggaggaactagctcagacccggatctattggcagaaagaaaagaagatggtgctgaccatgatgtccggcgacatgaacatttggccagagtacaagaaccgcacaattttcgacattacaaacaacctctctattgtgattctggctctcaggcctagcgacgagggcacatacgagtgcgtggtgctcaagtacgagaaggacgctttcaagcgggagcacctcgctgaggtgaccctgtccgtgaaggccgacttccctactccatctatctccgacttcgagatccctacctctaacatcaggcggatcatctgcagcacctccggcggcttcccagagcctcacctctcttggctggagaacggcgaggagctgaacgctatcaacaccacagtgtctcaggaccctgagaccgagctgtacgccgtgtcctctaagctcgacttcaacatgaccacaaaccactccttcatgtgcctgatcaagtacggccacctgagagtgaaccagacattcaactggaacacaaccaagcaggagcacttcccataagtcgac

SEQ ID NO: 114 - protein, 691 aa, Artificial sequence
  synthesized
  misc_feature: The amino acid sequence of heavy chain (BY12.7h) of conjugate by12.7
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 115 - DNA, 738 bp, Artificial sequence
  synthesized
  misc_feature: The light chain (BY20.2l) nucleotide sequence of anti-GPC-3 antibody by20.2
ctcgaggccaccatggagaccgacacactcctcctgtgggtgctgctgctgtgggtgcctggctccactggcgacgtggtgatgacacagtctccactgagcctccctgtgacaccaggcgagcccgcctcgatctcttgccggtcctctcagagcctcgtgcactctaacagaaacacatacctgcactggtacttacagaagcccggccagtcccctcagctgctgatctacaaggtgtccaacagattcagcggcgtgcccgatcgtttcagcggcagcggctccggcacagacttcacactgaaaatttcacgtgtggaggctgaggacgtgggcgtgtactactgctctcagaacacccacgtgccacctacattcggccagggcacaaagctcgaaatcaagagaaccgtggccgccccatccgtgttcatcttcccaccatccgacgagcagctcaagtctggcaccgcgtcagtggtgtgcctgctgaacaacttctaccctagggaggctaaggtgcagtggaaggtggacaacgctttgcagtctggcaactcccaggagtctgtgaccgagcaggactctaaggactccacatacagcctgtcctctacactcaccctgtccaaggccgactacgagaagcacaaggtgtacgcttgcgaggtgacccaccagggcctgtcttctccagtgacaaagtctttcaaccggggcgagtgctaagaattc

SEQ ID NO: 116 - protein, 239 aa, Artificial sequence
  synthesized
  misc_feature: The amino acid sequence of light chain (BY20.2l) of anti-GPC-3 antibody by20.2
METDTLLLWVLLLWVPGSTGDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - DNA, 1416 bp, Artificial sequence
  synthesized
  misc_feature: Heavy chain (BY20.2h) nucleotide sequence of anti-GPC-3 antibody by20.2
tctagagccaccatggagaccgacaccctgctgctgtgggtgctgctcctgtgggtgcctggctccacaggccaggtgcagctcgtgcagtccggcgccgaggtgaagaagcccggcgcttctgtgaaggtgtcttgcaaggcctcaggctacacattcaccgactacgagatgcactgggtgaggcaggctcctggccagggcctggagtggatgggcgctctcgacccaaagaccggcgacaccgcttactctcagaagttcaagggccgggtgacactgaccgccgacaagagcacatctaccgcttacatggagctatcatctctgacctccgaggacaccgccgtgtactactgcaccagattctactcttacacatactggggccagggcacactcgtgacagtgtctagcgcctcgacaaagggcccatctgtgttcccactcgctccatcttctaagtccacatctggcggcaccgccgctctcggctgcctcgtgaaggactacttccccgagcctgtgacagtgtcttggaacagcggcgccctcacatccggcgtgcacaccttccccgctgtgttgcagtctagcggcctgtactccctgagcagcgtggtgacagtgccatcctcttctctgggcacccagacatacatctgcaacgtgaaccacaagcctagcaacacaaaggtggacaagaaggtggagcctaagtcttgcgacaagacccacacatgcccaccttgccccgcccctgagctgctgggcggcccatctgtgttcctgttccctccaaagccaaaggacaccctcatgatcagcagaaccccagaggtgacatgcgtggtggtggacgtgtctcacgaggaccccgaggtgaagttcaactggtacgtggacggcgtggaggtgcacaacgctaagaccaagccacgggaggagcagtacaactctacctacagagtggtgtccgtgctcacagtgctgcaccaggactggctgaacggcaaggagtacaagtgcaaggtgtccaacaaggccctgcccgctcctatcgaaaaaacaatcagcaaggctaagggccagccacgggagcctcaggtgtacacactgccaccttccagggacgagctgaccaagaaccaggtgtccctcacatgcctcgtgaagggcttctaccctagcgacatcgccgtggagtgggagtctaacggccagccagagaacaactacaagaccacccctccagtgctcgactccgacggctctttcttcctgtactctaagctgaccgtggacaagtcacgatggcagcagggcaacgtgttcagctgctccgtgatgcacgaggctctccacaaccactacacccagaagtccctgagcctgtctcctggcaagtaagtcgac

SEQ ID NO: 118 - protein, 465 aa, Artificial sequence
  synthesized
  misc_feature: The amino acid sequence of heavy chain (BY20.2h) of anti-GPC-3 antibody by20.2
METDTLLLWVLLLWVPGSTGQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - DNA, 2085 bp, Artificial sequence
  synthesized
  misc_feature: Heavy chain nucleotide sequence of conjugate BY20.3
tctagagccaccatggagaccgacacactcctcctgtgggtgctgctgctgtgggtgcctggctccactggccaggtgcagctcgtgcagtccggcgccgaggtgaagaagcccggcgcttctgtgaaggtgtcttgcaaggcctcaggctacacattcaccgactacgagatgcactgggtgaggcaggctcctggccagggcctggagtggatgggcgctctcgacccaaagaccggcgacaccgcttactctcagaagttcaagggccgggtgacactgaccgccgacaagagcacatctaccgcttacatggagctatcatctctgacctccgaggacaccgccgtgtactactgcaccagattctactcttacacatactggggccagggcacactcgtgacagtgtctagcgcctcgacaaagggcccatctgtgttcccactcgctccatcttctaagtccacatctggcggcaccgccgctctcggctgcctcgtgaaggactacttccccgagcctgtgacagtgtcttggaacagcggcgccctcacatccggcgtgcacaccttccccgctgtgttgcagtctagcggcctgtactccctgagcagcgtggtgacagtgccatcctcttctctgggcacccagacatacatctgcaacgtgaaccacaagcctagcaacacaaaggtggacaagaaggtggagcctaagtcttgcgacaagacccacacatgcccaccttgccccgcccctgagctgctgggcggcccatctgtgttcctgttccctccaaagccaaaggacaccctcatgatcagcagaaccccagaggtgacatgcgtggtggtggacgtgtctcacgaggaccccgaggtgaagttcaactggtacgtggacggcgtggaggtgcacaacgctaagaccaagccacgggaggagcagtacaactctacctacagagtggtgtccgtgctcacagtgctgcaccaggactggctgaacggcaaggagtacaagtgcaaggtgtccaacaaggccctgcccgctcctatcgaaaaaacaatcagcaaggctaagggccagccacgggagcctcaggtgtacacactgccaccttccagggacgagctgaccaagaaccaggtgtccctcacatgcctcgtgaagggcttctaccctagcgacatcgccgtggagtgggagtctaacggccagccagagaacaactacaagaccacccctccagtgctcgactccgacggctctttcttcctgtactctaagctgaccgtggacaagtcaagggaacgatggcagcagggcaacgtgttcagctgctccgtgatgcacgaggctctccacaaccactacacccagaagtccctgagcctgtctcctggcaagggcggcggaggatctggcggcggaggcagtggaggcggcggaagtgtgatccatgtaacaaaagaagtgaaggaagtggctacactctcttgcggccacaacgtgtccgtggaggaactagctcagacccggatctattggcagaaagaaaagaagatggtgctgaccatgatgtccggcgacatgaacatttggccagagtacaagaaccgcacaattttcgacattacaaacaacctctctattgtgattctggctctcaggcctagcgacgagggcacatacgagtgcgtggtgctcaagtacgagaaggacgctttcaagcgggagcacctcgctgaggtgaccctgtccgtgaaggccgacttccctactccatctatctccgacttcgagatccctacctctaacatcaggcggatcatctgcagcacctccggcggcttcccagagcctcacctctcttggctggagaacggcgaggagctgaacgctatcaacaccacagtgtctcaggaccctgagaccgagctgtacgccgtgtcctctaagctcgacttcaacatgaccacaaaccactccttcatgtgcctgatcaagtacggccacctgagagtgaaccagacattcaactggaacacaaccaagcaggagcacttcccataagtcgac

SEQ ID NO: 120 - protein, 686 aa, Artificial sequence
  synthesized
  misc_feature: Heavy chain amino acid sequence of conjugate BY20.3
METDTLLLWVLLLWVPGSTGQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 121 - DNA, 723 bp, Artificial sequence
  synthesized
  misc_feature: Nucleotide sequence of light chain (BY43L) of anti-trop-2 antibody BY43
ctcgaggccaccatggagaccgatactctgctgctctgggtgctgctgctgtgggtccccggctccaccggcgacatccagctgacccagtcccctagctccctcagcgcgtcagtgggcgaccgggtgagcatcacatgcaaggcttctcaggacgtgtctatcgccgtggcttggtatcagcagaagccaggcaaggccccaaagctgctgatctactccgcctcttacagatacaccggcgtgcccgacagattcagcggctctggctccggcaccgacttcacactgacaatctctagcttgcaacccgaggacttcgccgtgtactactgccagcagcactacatcacaccactgacattcggcgctggcacaaaggtggagatcaagagaacagtggccgctcctagcgtgttcatcttcccaccatccgacgagcagctgaagagcggcaccgcttccgtggtgtgcctgctgaacaacttctaccctagggaggctaaggtgcagtggaaggtggacaacgccctacaatctggcaactctcaggagtctgtgacagagcaggactctaaggactccacatactctctgtcttctaccctcacactgtccaaggccgactacgagaagcacaaggtgtacgcttgcgaggtgacccaccagggcctgtcctctcctgtgaccaagtctttcaaccggggcgagtgctaagaattc

SEQ ID NO: 122 - protein, 234 aa, Artificial sequence
  synthesized
  misc_feature: Amino acid sequence of light chain (BY43L) of anti-trop-2 antibody BY43
METDTLLLWVLLLWVPGSTGDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 1437 bp, Artificial sequence
  synthesized
  misc_feature: Heavy chain nucleotide sequence of anti-trop-2 antibody BY43
tctagagccaccatggaaaccgacacactgctgctgtgggtcctgctgctgtgggtgccaggctctacaggccaggtgcagctgcaacagtccggcagcgagctgaagaagccaggcgcaagcgtgaaggtgtcttgcaaggcatctggctacacattcaccaactacggcatgaactgggtgaagcaggctcctggccagggcctcaagtggatgggctggatcaacacatacacaggcgagccaacatacaccgacgacttcaagggcagattcgctttcagcctcgacacatccgtgagcaccgcttacctccagatcagctctctgaaggccgacgacaccgccgtgtacttctgcgcgagaggcggcttcggcagctcttactggtacttcgacgtgtggggccagggctctctcgtgacagtgtccagcgcatcaacaaagggcccatctgtgttcccactcgctccatcttctaagtctacatccggcggcaccgccgccctcggctgcctcgtgaaggactacttccctgagcctgtgaccgtgtcttggaactctggcgccctcacatccggcgtgcacacattccccgctgtgctccagtctagcggcctgtactctctgtcctctgtggtgacagtgccatctagctccctcggcacccagacctacatctgcaacgtgaaccacaagccttctaacacaaaggtggacaagagagtggagcctaagtcttgcgacaagacccacacatgcccaccttgccccgcccccgagctgctgggcggcccatctgtgttcctgttccctccaaagccaaaggacacactcatgatcagcagaaccccagaggtgacatgcgtggtggtggacgtgtctcacgaggaccccgaggtgaagttcaactggtacgtggacggcgtggaggtgcacaacgctaagaccaagccacgggaggagcagtacaactctacctaccgggtggtgagcgtgctgacagtgctgcaccaggactggctcaacggcaaggagtacaagtgcaaggtgtctaacaaggccctgcccgctcctatcgaaaagaccatcagcaaggctaagggccagcctagagagcctcaggtgtacacactgccaccttctcgggaggagatgacaaagaaccaggtgtccctgacatgcctcgtgaagggcttctaccctagcgacatcgccgtggagtgggagtctaacggccagcccgagaacaactacaagaccaccccacctgtgctcgactccgacggctctttcttcctgtactctaagctgacagtggacaagtccagatggcagcagggcaacgtgttctcttgctccgtgatgcacgaggctctccacaaccactacacccagaagtccctgagcctgtcccctggcaagggataagtcgac

SEQ ID NO: 124 - protein, 471 aa, Artificial sequence
  synthesized
  misc_feature: Heavy chain amino acid sequence of anti-trop-2 antibody BY43
METDTLLLWVLLLWVPGSTGQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 2085 bp, Artificial sequence; identical to SEQ ID NO: 119
  synthesized
  misc_feature: Heavy chain nucleotide sequence of conjugate BY43.2

SEQ ID NO: 126 - protein, 686 aa, Artificial sequence; identical to SEQ ID NO: 120
  synthesized
  misc_feature: Heavy chain amino acid sequence of conjugate BY43.2

SEQ ID NO: 127 - DNA, 2046 bp, Artificial sequence
  synthesized
  misc_feature: Nucleotide sequence of conjugate BY24.22 (VEGFR-Fc-CD80, IgG4)
ctcgaggccaccatggagaccgacacactcctcctgtgggtgctgctgctgtgggtgcctggctccactggcgcttccgacaccggccgccctttcgtggagatgtactccgagatccctgagatcatccacatgaccgagggccgcgagctggtgatcccttgccgcgtgacctcccctaacatcaccgtgaccctgaagaagttccctctggacaccctgatccctgacggcaagcgcatcatctgggactcccgcaagggcttcatcatctccaacgccacctacaaggagatcggcctgctgacctgcgaggccaccgtgaacggccacctgtacaagaccaactacctgacccaccgccagaccaacaccatcatcgacgtggtgctgtccccttcccacggcatcgagctgtccgtgggcgagaagctggtgctgaactgcaccgcccgcaccgagctgaacgtgggcatcgacttcaactgggagtacccttcctccaagcaccagcacaagaagctggtgaaccgcgacctgaagacccagtccggctccgagatgaagaagttcctgtccaccctgaccatcgacggcgtgacccgctccgaccagggcctgtacacctgcgccgcctcctccggcctgatgaccaagaagaactccaccttcgtgcgcgtgcacgaggagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggcaagggaggcggaggatctggcggcggaggcagtggaggcggcggaagtgtcatccatgtgaccaaagaggtcaaagaagtcgcaactctgtcttgcggacacaacgtctccgtcgaagaactcgcccagactagaatatattggcagaaggaaaagaagatggtgctcaccatgatgtccggcgatatgaacatttggcctgagtacaagaaccggacaatcttcgatattactaataacctgagtatcgtgattctcgctctgcgccctagcgacgagggcacatacgagtgtgtggtgctgaagtacgagaaggatgcattcaagcgggagcacctcgcagaggtgacactctccgtgaaggccgacttcccaaccccatctatctccgacttcgagatccctacctctaacatcaggcggatcatctgcagcacctccggcggcttcccagagcctcacctctcttggctggagaacggcgaggagctgaacgctatcaacaccacagtgtctcaggaccctgagaccgagctgtacgccgtgtcctctaagctcgacttcaacatgaccacaaaccactccttcatgtgcctgatcaagtacggccacctgagagtgaaccagacattcaactggaacacaaccaagcaggagcacttcccataagaattc

SEQ ID NO: 128 - protein, 675 aa, Artificial sequence
  synthesized
  misc_feature: The amino acid sequence of the fusion protein BY24.22 (VEGFR-Fc-CD80, IgG4)
METDTLLLWVLLLWVPGSTGASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 129 - DNA, 780 bp, Artificial sequence
  synthesized
  misc_feature: The Light chain nucleotide sequence of conjugate By 41.6 (extracellular domain of TGF   receptor+light chain constant region, extracellular region of +CD80)
ctcgaggccaccatggagaccgacacactcctcctgtgggtgctgctgctgtgggtgcctggctccactggcgtgactgacaacaacggcgctgtgaagttcccacagctctgcaagttctgcgacgtgagattctctacttgcgacaaccagaagtcttgcatgtctaactgctccatcactagcatttgcgagaagcctcaggaggtgtgcgtggccgtgtggcgcaagaacgacgagaacattacactggagaccgtgtgccacgacccaaagctgccataccacgacttcatcttagaggacgccgcatctcctaagtgcattatgaaggagaagaagaagcccggcgagactttcttcatgtgctcatgtagctctgacgagtgcaacgacaacattatcttctccgaggagtacaacacatctaaccctggaggcggaggatctagaaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaagaattc

SEQ ID NO: 130 - protein, 253 aa, Artificial sequence
  synthesized
  misc_feature: The light chain amino acid sequence of conjugate By 41.6 (extracellular domain of TGF   receptor+light chain constant region, extracellular region of +CD80)
METDTLLLWVLLLWVPGSTGVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - DNA, 2106 bp, Artificial sequence
  synthesized
  misc_feature: Heavy chain nucleotide sequence of conjugate BY41.6(TGF receptor extracellular domain +IgG heavy chain constant region IgG4+CD80 extracellular region)
tctagagccaccatggagaccgacacactcctcctgtgggtgctgctgctgtgggtgcctggctccactggcgtgactgacaacaacggcgctgtgaagttcccacagctctgcaagttctgcgacgtgagattctctacttgcgacaaccagaagtcttgcatgtctaactgctccatcactagcatttgcgagaagcctcaggaggtgtgcgtggccgtgtggcgcaagaacgacgagaacattacactggagaccgtgtgccacgacccaaagctgccataccacgacttcatcttagaggacgccgcatctcctaagtgcattatgaaggagaagaagaagcccggcgagactttcttcatgtgctcatgtagctctgacgagtgcaacgacaacattatcttctccgaggagtacaacacatctaaccctggaggcggaggatcttccgcctccaccaagggcccttccgtgttccctctggccccttgctcccgctccacctccgagtccaccgccgccctgggctgcctggtgaaggactacttccctgagcctgtgaccgtgtcctggaactccggcgccctgacctccggcgtgcacaccttccctgccgtgctgcagtcctccggcctgtactccctgtcctccgtggtgaccgtgccttcctcctccctgggcaccaagacctacacctgcaacgtggaccacaagccttccaacaccaaggtggacaagcgcgtggagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggcaagggaggcggaggatctggcggcggaggcagtggaggcggcggaagtgtgatccatgtaacaaaagaagtgaaggaagtggctacactctcttgcggccacaacgtgtccgtggaggaactagctcagacccggatctattggcagaaagaaaagaagatggtgctgaccatgatgtccggcgacatgaacatttggccagagtacaagaaccgcacaattttcgacattacaaacaacctctctattgtgattctggctctcaggcctagcgacgagggcacatacgagtgcgtggtgctcaagtacgagaaggacgctttcaagcgggagcacctcgctgaggtgaccctgtccgtgaaggccgacttccctactccatctatctccgacttcgagatccctacctctaacatcaggcggatcatctgcagcacctccggcggcttcccagagcctcacctctcttggctggagaacggcgaggagctgaacgctatcaacaccacagtgtctcaggaccctgagaccgagctgtacgccgtgtcctctaagctcgacttcaacatgaccacaaaccactccttcatgtgcctgatcaagtacggccacctgagagtgaaccagacattcaactggaacacaaccaagcaggagcacttcccataagtcgac

SEQ ID NO: 132 - protein, 695 aa, Artificial sequence
  synthesized
  misc_feature: Heavy-chain amino acid sequence of conjugate By 41.6 (extracellular domain of TGF   receptor +IgG heavy-chain constant region, extracellular region of IgG4+CD80)
METDTLLLWVLLLWVPGSTGVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPGGGGSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 133 - protein, 288 aa, Artificial sequence
  synthesized
  misc_feature: Amino acid sequence of human CD80 (also known as “human B7.1”)
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 134 - protein, 329 aa, Artificial sequence
  synthesized
  misc_feature: Amino acid sequence of human CD86 (also known as “human B7.2”)
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 135 - protein, 302 aa, Artificial sequence
  synthesized
  misc_feature: Amino acid sequence of human ICOSL
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 136 - protein, 306 aa, Artificial sequence
  synthesized
  misc_feature: Amino acid sequence of mouse CD80(B7.1)
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 137 - protein, 102 aa, Artificial sequence
  synthesized
  misc_feature: Amino acid sequence of human CD80 extracellular domain IgC
DFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 138 - protein, 671 aa, Artificial sequence
  synthesized
  misc_feature: Amino acid sequence of anti-HER2/neu antibody heavy chain+extracellular domain of +CD80
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 139 - protein, 214 aa, Artificial sequence
  synthesized
  misc_feature: Amino acid sequence of light chain of anti-HER2/neu antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 674 aa, Artificial sequence
  synthesized
  misc_feature: Amino acid sequence of anti-VEGF antibody heavy chain +CD80 extracellular domain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 141 - protein, 214 aa, Artificial sequence
  synthesized
  misc_feature: Amino acid sequence of anti-VEGF antibody light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 671 aa, Artificial sequence
  synthesized
  misc_feature: Amino acid sequence of anti-CD20 antibody heavy chain+extracellular domain of CD80
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 143 - protein, 213 aa, Artificial sequence
  synthesized
  misc_feature: Amino acid sequence of anti-CD20 antibody light chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 672 aa, Artificial sequence
  synthesized
  misc_feature: Amino acid sequence of anti-Trop-2 antibody heavy chain+extracellular domain of CD80
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 145 - protein, 214 aa, Artificial sequence
  synthesized
  misc_feature: Amino acid sequence of light chain of anti-Trop-2 antibody
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 661 aa, Artificial sequence
  synthesized
  misc_feature: Amino acid sequence of anti-PD1 antibody heavy chain+extracellular domain of CD80
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 147 - protein, 214 aa, Artificial sequence; identical to SEQ ID NO: 141
  synthesized
  misc_feature: Amino acid sequence of anti-PD1 antibody light chain

SEQ ID NO: 148 - protein, 669 aa, Artificial sequence
  synthesized
  misc_feature: Amino acid sequence of anti-PD-L1 antibody heavy chain+extracellular domain of CD80
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 149 - protein, 214 aa, Artificial sequence
  synthesized
  misc_feature: Amino acid sequence of light chain of anti-PD-L1 antibody
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 666 aa, Artificial sequence
  synthesized
  misc_feature: Amino acid sequence of anti-phosphatidylinositol proteoglycan 3 antibody heavy chain+extracellular domain of CD80
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 151 - protein, 219 aa, Artificial sequence
  synthesized
  misc_feature: Amino acid sequence of anti-phosphatidylinositol proteoglycan 3 antibody light chain
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 669 aa, Artificial sequence
  synthesized
  misc_feature: Amino acid sequence of anti-CTLA-4 anti-heavy chain+extracellular domain of CD80
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 153 - protein, 215 aa, Artificial sequence
  synthesized
  misc_feature: Amino acid sequence of light chain of anti-CTLA-4 antibody
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 670 aa, Artificial sequence
  synthesized
  misc_feature: Amino acid sequence of anti-EGFR antibody heavy chain+extracellular domain of CD80
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 155 - protein, 214 aa, Artificial sequence
  synthesized
  misc_feature: Amino acid sequence of light chain of anti-EGFR antibody
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 665 aa, Artificial sequence
  synthesized
  misc_feature: Amino acid sequence of anti-ALK-1 antibody heavy chain+extracellular domain of CD80
QVQLQESGPGLVKPSQTLSLTCTVSGGSISSGEYYWNWIRQHPGKGLEWIGYIYYSGSTYYNPSLKSRVTISVDTSKNQFSLKLSSVTAADTAVYYCARESVAGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 157 - protein, 215 aa, Artificial sequence
  synthesized
  misc_feature: Amino acid sequence of light chain of anti-ALK-1 antibody
EIVLTQSPGTLSLSPGERATLSCRASQSVSSSYLAWYQQKPGQAPRLLIYGTSSRATGIPDRFSGSGSGTDFTLTISRLEPEDFAVYYCQQYGSSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 662 aa, Artificial sequence
  synthesized
  misc_feature: Amino acid sequence of anti-CD30 antibody heavy chain +extracellular domain of CD80
QVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 159 - protein, 214 aa, Artificial sequence
  synthesized
  misc_feature: Amino acid sequence of anti-CD30 antibody light chain
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 664 aa, Artificial sequence
  synthesized
  misc_feature: Amino acid sequence of anti-CD33 antibody heavy chain+extracellular domain of CD80
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 161 - protein, 218 aa, Artificial sequence
  synthesized
  misc_feature: Amino acid sequence of anti-CD33 antibody light chain
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 672 aa, Artificial sequence
  synthesized
  misc_feature: Amino acid sequence of anti-CEA antibody heavy chain + extracellular domain of CD80
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 163 - protein, 215 aa, Artificial sequence
  synthesized
  misc_feature: Amino acid sequence of anti-CEA antibody light chain
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 669 aa, Artificial sequence
  synthesized
  misc_feature: Amino acid sequence of anti-IGF1R antibody heavy chain+extracellular domain of CD80
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 165 - protein, 215 aa, Artificial sequence
  synthesized
  misc_feature: Amino acid sequence of light chain of anti-IGF1R antibody
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 665 aa, Artificial sequence
  synthesized
  misc_feature: Amino acid sequence of anti-CD47 antibody heavy chain +extracellular domain of CD80
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 167 - protein, 219 aa, Artificial sequence
  synthesized
  misc_feature: Amino acid sequence of anti-CD47 antibody light chain
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 661 aa, Artificial sequence
  synthesized
  misc_feature: Amino acid sequence of anti-TIM-3 antibody heavy chain+extracellular domain of CD80
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 169 - protein, 214 aa, Artificial sequence
  synthesized
  misc_feature: Amino acid sequence of anti-TIM-3 antibody light chain
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 668 aa, Artificial sequence
  synthesized
  misc_feature: Amino acid sequence of anti-LAG-3 antibody heavy chain+extracellular domain of CD80
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 171 - protein, 214 aa, Artificial sequence
  synthesized
  misc_feature: Amino acid sequence of light chain of anti-LAG-3 antibody
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 677 aa, Artificial sequence
  synthesized
  misc_feature: Amino acid sequence of anti-TIGIT antibody heavy chain +extracellular domain of CD80
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 173 - protein, 220 aa, Artificial sequence
  synthesized
  misc_feature: Amino acid sequence of light chain of anti-TIGIT antibody
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 669 aa, Artificial sequence
  synthesized
  misc_feature: Amino acid sequence of anti-4-1BB antibody heavy chain+extracellular domain of CD80
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 175 - protein, 216 aa, Artificial sequence
  synthesized
  misc_feature: Amino acid sequence of light chain of anti-4-1BB antibody
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 665 aa, Artificial sequence
  synthesized
  misc_feature: Amino acid sequence of anti-OX40 antibody heavy chain +extracellular domain of CD80
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 177 - protein, 214 aa, Artificial sequence
  synthesized
  misc_feature: Amino acid sequence of light chain of anti-OX40 antibody
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 668 aa, Artificial sequence
  synthesized
  misc_feature: Amino acid sequence of anti-ICOS antibody heavy chain +extracellular domain of CD80
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 179 - protein, 218 aa, Artificial sequence
  synthesized
  misc_feature: Amino acid sequence of light chain of anti-ICOS antibody
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 670 aa, Artificial sequence
  synthesized
  misc_feature: Amino acid sequence of anti-CD27 antibody heavy chain+extracellular domain of CD80
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 181 - protein, 214 aa, Artificial sequence
  synthesized
  misc_feature: Amino acid sequence of light chain of anti-CD27 antibody
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 671 aa, Artificial sequence
  synthesized
  misc_feature: Amino acid sequence of extracellular domain of transforming factor   receptor +IgG heavy chain constant domain +extracellular domain of CD80
VTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 183 - protein, 228 aa, Artificial sequence
  synthesized
  misc_feature: Amino acid sequence of extracellular domain+light chain constant domain of transforming factor receptor
VTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 580 aa, Artificial sequence
  synthesized
  misc_feature: The amino acid sequence of CD24 extracellular domain +IgG heavy chain constant domain +CD80 extracellular domain
SETTTGTSSNSSQSTSNSGLAPNPTNATTKSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 185 - protein, 137 aa, Artificial sequence
  synthesized
  misc_feature: Amino acid sequence of CD24 extracellular region+light chain constant region
SETTTGTSSNSSQSTSNSGLAPNPTNATTKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 186 - protein, 656 aa, Artificial sequence
  synthesized
  misc_feature: Amino Acid Sequence of ALK1 Extracellular Region +IgG Heavy Chain Constant Region + Extracellular Region of CD80
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 187 - protein, 213 aa, Artificial sequence
  synthesized
  misc_feature: Amino acid sequence of ALK1 extracellular region+light chain constant region
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 684 aa, Artificial sequence
  synthesized
  misc_feature: Amino Acid Sequence of FZD8 Extracellular Region +IgG Heavy Chain Constant Region +Extracellular Region of CD80
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 189 - protein, 241 aa, Artificial sequence
  synthesized
  misc_feature: Amino acid sequence of FZD8 extracellular domain+light chain constant domain
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - protein, 673 aa, Artificial sequence
  synthesized
  misc_feature: Amino acid sequence of CD47 extracellular domain +IgG heavy chain constant domain +CD80 extracellular domain
GPGSEEEVQIIQPDKSVSVAAGESAILHCTITSLFPVGPIQWFRGAGPARVLIYNQRQGPFPRVTTISETTRRENMDFSISISNITPADAGTYYCIKFRKGSPDTEFKSGAGTELSVRAKPSASSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 191 - protein, 230 aa, Artificial sequence
  synthesized
  misc_feature: Amino acid sequence of CD47 extracellular domain+light chain constant domain
GPGSEEEVQIIQPDKSVSVAAGESAILHCTITSLFPVGPIQWFRGAGPARVLIYNQRQGPFPRVTTISETTRRENMDFSISISNITPADAGTYYCIKFRKGSPDTEFKSGAGTELSVRAKPSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - protein, 655 aa, Artificial sequence
  synthesized
  misc_feature: Amino acid sequence of VEGF receptor extracellular domain +Fc +CD80 extracellular domain
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 193 - protein, 779 aa, Artificial sequence
  synthesized
  misc_feature: Amino acid sequence of interleukin-1 receptor helper protein extracellular domain +Fc +CD80 extracellular domain
GAARSCRFRGRHYKREFRLEGEPVALRCPQVPYWLWASVSPRINLTWHKNDSARTVPGEEETRMWAQDGALWLLPALQEDSGTYVCTTRNASYCDKMSIEFRVFENTDAFLPFISYPQILTLSTSGVLVCPDLSEFTRDKTDVKIQWYRDSLLLDKDNEKFLSVRGTTHLLVHDVAQEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 194 - protein, 596 aa, Artificial sequence
  synthesized
  misc_feature: Amino acid sequence of CD95 protein extracellular domain +FC+CD80 extracellular domain
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 195 - protein, 583 aa, Artificial sequence
  synthesized
  misc_feature: Amino acid sequence of IL-2+Fc+ CD80 extracellular domain
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 196 - protein, 605 aa, Artificial sequence
  synthesized
  misc_feature: Amino acid sequence of IL-7+Fc+ CD80 extracellular domain
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 197 - protein, 609 aa, Artificial sequence
  synthesized
  misc_feature: Amino acid sequence of IL-33+Fc+ CD80 extracellular domain
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSETESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 198 - protein, 564 aa, Artificial sequence
  synthesized
  misc_feature: Amino acid sequence of IL-13+Fc+ CD80 extracellular domain
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 199 - protein, 669 aa, Artificial sequence
  synthesized
  misc_feature: Amino acid sequence of anti-CLDN18.2 antibody heavy chain+extracellular domain of CD80
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 200 - protein, 220 aa, Artificial sequence
  synthesized
  misc_feature: Amino acid sequence of light chain of anti-CLDN18.2 antibody
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 666 aa, Artificial sequence
  synthesized
  misc_feature: Amino acid sequence of anti-IL-17 antibody heavy chain+extracellular domain of CD80
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 202 - protein, 219 aa, Artificial sequence
  synthesized
  misc_feature: Amino acid sequence of light chain of anti-IL-17 antibody
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 585 aa, Artificial sequence
  synthesized
  misc_feature: Amino acid sequence of NKG2D protein extracellular domain +Fc +CD80 extracellular domain
NSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFP

SEQ ID NO: 204 - protein, 111 aa, Artificial sequence
  synthesized
  misc_feature: Zoloxizumab VH
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQG

SEQ ID NO: 205 - protein, 107 aa, Artificial sequence
  synthesized
  misc_feature: Zoloxizumab VL
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSG

SEQ ID NO: 206 - protein, 137 aa, Artificial sequence
  synthesized
  misc_feature: NKG2D
NSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 207 - protein, 192 aa, Artificial sequence
  synthesized
  misc_feature: AXL Extracellular region
ESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLP

SEQ ID NO: 208 - protein, 133 aa, Artificial sequence [sequence omitted]

SEQ ID NO: 209 - protein, 306 aa, Artificial sequence
  synthesized
  misc_feature: IL-12 P40 subunit
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 210 - protein, 197 aa, Artificial sequence
  synthesized
  misc_feature: IL-12 P35 subunit
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 211 - protein, 470 aa, Artificial sequence
  synthesized
  misc_feature: The amino acid sequence of fusion protein BY24.39
METDTLLLWVLLLWVPGSTGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTAGGFPEPHLSWLENGEELNAINTTVIQDPETELYAVSSKLDFNMTTNHSFMCLIKYGHLRVNQTFNWNTTKQEHFP